(19) United States
(12) Patent Application Publication
(10) Pub. No.: US 2026/0317740 A1
(43) Pub. Date: Oct. 15, 2026

(54) NUCLEIC ACIDS ENCODING IMPROVED LIPASE PROTEINS

(71) Applicant: BAYER AKTIENGESELLSCHAFT, LEVERKUSEN (DE)

(72) Inventors: Christian PITZLER, Leverkussen (DE); Oliver KENSCH, Leverkusen (DE); Florian RICHTER, Leverkusen (DE); Marcus LUDWIG, Leverkusen (DE)

(21) Appl. No.: 19/105,440

(22) PCT Filed: Aug. 17, 2023
(86) PCT No.: PCT/EP2023/072709

§ 371 Date: Feb. 21, 2025

(30) Foreign Application Priority Data
Aug. 23, 2022 (EP) 22191615.8

(51) Int. Cl. C12N 9/20; C12P 13/00

(52) U.S. Cl. CPC C12N 9/20; C12P 13/001

(57) ABSTRACT

The present invention concerns proteins having improved lipase activity, nucleic acid molecules encoding respective proteins having improved lipase activity and methods for stereo-selective synthesis of chiral amines or increasing of chiral amines isomers in enantiomer mixtures.

The present invention concerns proteins having improved lipase activity, nucleic acid molecules encoding respective proteins having improved lipase activity and methods for stereo-selective synthesis of chiral amines or increasing of chiral amines isomers in enantiomer mixtures.

Biocatalysis can be based on enzymes available in nature. More often a desire to produce a specific product creates demand for a specific enzyme, which is adapted to allow for economically feasible production of the desired product in large scale. Enzyme engineering is one option for optimizing enzymes towards the economical production of a given product.

Amines are ubiquitous in nature not only as parts of nucleic acids but have e.g. also great importance as tissue hormones (e.g. histamine and serotonin), neurotransmitters (e.g. dopamine and noradrenaline), as building blocks for active pharmaceutical ingredients or agricultural products. The absolute configuration of the stereocenters of chiral amines is crucial for the synthesis of the herbicidal active agents. In the production of a desired target molecule, generation of correct chirality is often a challenge.

The use of enzymes, for example lipases, as biocatalysts is of great importance for the production of chiral compounds. Kirchner et al. reported two lipases that act as highly stereoselective, practical catalysts in nearly anhydrous organic solvents. Under such “unnatural” conditions the enzymes can asymmetrically catalyze reactions of esterification and transesterification which are not feasible in aqueous solutions because of the domination of hydrolysis. As a result, a number of optically active alcohols, carboxylic acids, and esters have been prepared on a gram scale (Gerald Kirchner, Mark P. Scollar, and Alexander M. Klibanov, J. Am. Chem. Soc. (1985), 107, 7072-7076). Slotema et al. describe the development of an economically pertinent process for the lipase-catalyzed synthesis of amides. They produced oleamide via direct Candida antarctica lipase B catalyzed amidation of oleic acid with ammonia in 2-methyl-2-butanol (Slotema W F, Sandoval G, Guieysse D, Straathof A J, Marty A. Economically pertinent continuous amide formation by direct lipase-catalyzed amidation with ammonia. Biotechnol Bioeng. (2003) 82(6):664-9. doi: 10.1002/bit.10613. PMID: 12673765). Ismail et al. describe enzymatic resolution of two chiral amines, 2-heptylamine and 2-phenylethylamine by Candida antarctica Lipase B, where different acyl donor reagents with NH, O and S moieties were tested (Hilda Ismail, Rute Madeira Lau, Fred van Rantwijk, Roger A. Sheldon; Fully Enzymatic Resolution of Chiral Amines: Acylation and Deacylation in the Presence of Candida antarctica Lipase B; Advanced Synthesis & Catalysis (2008) 350(10):1511-1516). A lipase-catalyzed dynamic kinetic resolution of chiral amines with the use of palladium as the racemization catalyst was reported by Reetz and Schimossek. They used a biocatalyst (lipase Candida antarctica) and a transition-metal catalyst (palladium) for making the dynamic kinetic resolution of racemic phenylethylamine possible. Conversion to the enantiomerically pure N-acylated form was reported to be 75-77% (ee=99%) (Manfred T. Reetz and Klaus Schimossek; Lipase-Catalyzed Dynamic Kinetic Resolution of Chiral Amines: Use of Palladium as the Racemization Catalyst; Chimia 50 (1996) 668-669). Sun et al. describe a lipase catalyzed reaction for selective amidation of phenylglycinol. They describe that enzymatic synthesis with Candida antarctica lipase B exhibited high regio-selectivity, and conversions and provide a promising alternative strategy for the synthesis of aromatic alkanolamides (Sun M., Nie K., Wang F. and Deng L.; Optimization of the Lipase-Catalyzed Selective Amidation of Phenylglycinol. (2020) Front. Bioeng. Biotechnol. 7:486.).

WO9728271A1 describes a method for the production of optically active amines that are useful intermediates for manufacturing pharmaceuticals or plant protection products. In a first step, suitable racemic amines are allowed to react in the presence of a lipase from Candida antarctica and optionally in the presence of a diluting agent with an ester and in a second step, the mixture obtained is separated

EP 0716712 B1 describes the lipase-catalyzed acylation of alcohols with diketenes, especially for the production of enantioselective acylated alcohols from racemic alcohols.

U.S. Pat. No. 5,512,454 discloses a process for preparing beta lactam antibiotic intermediates involving enzymatic acylation of 3-hydroxymethyl cephalosporins.

U.S. Pat. No. 5,902,738 is concerned with a process for making starting material compounds used for producing the corresponding vitamin A acylates. The process comprises the steps of firstly, reacting a compound with an acylating agent which is in a mixture comprising an organic solvent and a lipase which is present in a suspension, so as to form the compound and secondly, recovering the compound.

EP 2283144 B1 describes a process for producing sphingolipids by N-acetylation of lysophingolipids employing a lipase.

WO2012146935A1 discloses modified lipase variants, as well as polynucleotides and recombinant expression vectors encoding the lipase variant polypeptides, as well as methods for producing such lipase variants in selected bacterial and fungal host cells. The specified lipase variants have increased enzyme specificity or enhanced trans-selectivity. Further described are methods of their use for reducing or eliminating trans-fatty acids from substrates.

Although several improvements of lipases have been achieved so far, limitations arising during the asymmetric synthesis of amines or resolution of racemic amines, such as unfavorable equilibrium, substrate and product inhibition, poor thermostability, insufficient substrate specificity and sometimes low enantioselectivity of the lipase, still have to be overcome for an efficient production of a wide range of amines on industrial scale.

Thus, there is a need for further improvement of lipases, in particular in respect to production of desired acylated or carboxylated, enantiomerically enriched or pure products, preferably under specific and/or economically viable production processes improved further lipases are needed.

The present invention provides lipase variants comprising modifications in their amino acid sequence, these lipase variants having improved reaction kinetics, improved substrate acceptance e.g. improved selectivity and/or improved specific activity in comparison to the respective wild-type lipase. The lipase variants of the invention therefore enable the development of economically efficient production processes for acylated or carboxylated products in production methods of new acylated or carboxylated products or precursors of respective products not achievable by the use of the respective wild-type lipase.

The variant lipases described herein have advantages over known wild-type and other already known lipases. In particular, the modified or variant lipases described herein have the advantage that they can produce enantiomerically enriched or enantiomerically nearly pure or pure compounds better than the respective wild-type lipase.

SEQ ID No. 1 represents the amino acid sequence of a wild-type lipolytic protein. Said wild-type lipase is derived from an uncultured bacterium from an environmental sample derivable from GenPept (PDB) under accession No. QRD81023 (version ORD81023.1). In case of ambiguities regarding the amino acid sequence shown in SEQ ID No 1 and the sequence shown in above mentioned database entry, SEQ ID No 1 precedes.

Described herein are proteins having the activity of a lipolytic enzyme or lipase respectively, wherein the amino acid sequences of these proteins represent variants of a known protein having the activity of a lipolytic enzyme or lipase respectively. In particular, the amino acid sequence of proteins having the activity of a lipase described herein represent variants of the amino acid sequence represented by the amino acid shown in SEQ ID No. 1, wherein in the amino acid sequence shown under SEQ ID No. 1 at least the amino acid at position 186, the amino acid at position 280, the amino acid at position 312, the amino acid at position 3, the amino acid at position 29, the amino acid at position 17, the amino acid at position 4, the amino acid at position 18, the amino acid at position 202, the amino acid at position 301, the amino acid at position 309, the amino acid at position 31, the amino acid at position 111, the amino acid at position 85, the amino acid at position 8, the amino acid at position 79 or the amino acid at position 40 is different from the amino acid given at the respective amino acid position in the sequence shown under SEQ ID No. 1.

The term “variant” as used herein means subject-matter which is different from subject-matter known in the art. With respect to nucleic acid molecules and proteins, variants are understood to comprise a nucleic acid sequence or an amino acid sequence, respectively, which deviates from accordingly known sequences but encode a protein having the same function or catalyzing the same reaction e.g. the function of encoding a protein having the activity of an lipase. “Deviation” of nucleic acid molecule sequences and amino acid sequences from known nucleic acid sequences and protein sequences means that the sequences comprise substitutions (replacements) and/or deletions and/or insertions of nucleotides or amino acids, respectively, in comparison to the correspondingly known nucleic acid sequences or amino acid sequences.

A first embodiment of the invention concerns proteins having the activity of a lipase, wherein the proteins are encoded by an amino acid sequence having at least 80%, preferably 85%, more preferably 90%, further more preferably 95%, even more preferably 96%, even further more preferably 97%, particular preferably 98%, most preferably 99% identity with the amino acid sequence shown under SEQ ID No. 1 apart from that said amino acid sequence having at least 80%, preferably 85%, more preferably 90%, further more preferably 95%, even more preferably 96%, even further more preferably 97%, particular preferably 98%, most preferably 99% sequence identity with the amino acid sequence shown under SEQ ID No. 1 comprises a modification selected from the group consisting of
i. the amino acid at position 186 is different from L, preferably, the amino acid at position 186 is F, W, Y, E, D, Q, T, H, P, C, K, S, N, I, or V, more preferably, the amino acid at position 186 is F, W, Y, E, D or K, particular preferably, the amino acid at position 186 is W or Y, most preferably the amino acid at position 186 is Y;
ii. the amino acid at position 280 is different from L, preferably, the amino acid at position 280 is E, S, K, D or A, more preferably, the amino acid at position 280 is A;
iii. the amino acid at position 312 is different from P, preferably, the amino acid at position 312 is N, F, D, Q or K, more preferably, the amino acid at position 312 is N;
iv. the amino acid at position 3 is different from M, preferably, the amino acid at position 3 is L, Q or C, more preferably, the amino acid at position 3 is Q;
v. the amino acid at position 29 is different from N, preferably, the amino acid at position 29 is H, W or Y, more preferably, the amino acid at position 29 is H or W, most preferably, the amino acid at position 29 is H;
vi. the amino acid at position 17 is different from L, preferably, the amino acid at position 17 is P or T, more preferably, the amino acid at position 17 is P;
vii. the amino acid at position 4 is different from S, preferably, the amino acid at position 4 is P or L, more preferably, the amino acid at position 4 is P;
viii. the amino acid at position 18 is different from V, preferably, the amino acid at position 18 is A, T, C or S, more preferably, the amino acid at position 18 is A or C;
ix. the amino acid at position 202 is different from A, preferably, the amino acid at position 202 is Q or N. More preferably, the amino acid at position 202 is N;
x. the amino acid at position 301 is different from D, preferably, the amino acid at position 301 is A;
xi. the amino acid at position 309 is different from P, preferably, the amino acid at position 309 is C;
xii. the amino acid at position 31 is different from Q, preferably, the amino acid at position 31 is W;
xiii. the amino acid at position 111 is different from Q preferably, the amino acid at position 111 is E;
xiv. the amino acid at position 85 is different from W, preferably, the amino acid at position 85 is H;
xv. the amino acid at position 8 is different from K, preferably, the amino acid at position 8 is E;
xvi. the amino acid at position 79 is different from E, preferably, the amino acid at position 79 is S, I or W, more preferably, the amino acid at position 79 is S;
xvii. the amino acid at position 40 is different from K, preferably, the amino acid at position 40 is M.

The meaning of amino acid abbreviations A, C, D, E, F, G, H, I, K, L, M, N, P, Q, R, S, T V, W, Y is derivable herein below Table 2 under the paragraph sub-titled “Description of the Sequences”.

A further embodiment of the invention concerns proteins having the activity of a lipase, wherein the proteins are selected from the group consisting of
a) proteins comprising the amino acid sequence as shown in SEQ ID No. 1 apart from that the amino acid at position 186 is different from L;
b) proteins having an amino acid sequence having at least 80%, preferably 85%, more preferably 90%, further more preferably 95%, even more preferably 96%, even further more preferably 97%, particular preferably 98%, most preferably 99% identity with the amino acid sequence shown under a) given that the amino acid at position 186 is different from L.

Preferably, the amino acid at position 186 is F, W, Y, E, D, Q, T, H, P, C, K, S, N, I, or V. More preferably, the amino acid at position 186 is F, W, Y, E, D or K. Particular preferably, the amino acid at position 186 is W or Y. Most preferably, the amino acid at position 186 is Y.

An “amino acid corresponding to position x” in a first amino acid sequence (e.g. position 3 in SEQ ID No. 1) means herein that an amino acid of a second amino acid sequence, when compared with the first amino acid sequence, appears at position x of the first amino acid sequence in a pairwise sequence alignment of the first amino acid sequence with the second amino acid sequence in case the numbering of the amino acids of the second amino acid sequence differs from the amino acid numbering of the first amino acid sequence.

In the context of the present invention, the term “identity” in respect to sequence identity or sequences being identical to is to be understood as meaning the number of identical amino acids or nucleotides shared over the entire sequence length by a first nucleic or amino acid sequence with another (second) nucleic or amino acid sequence, respectively, expressed in percent.

“Sequence identity” can be determined by alignment of two amino acid or two nucleotide sequences using global or local alignment algorithms comprised for example in known software like GAP or BESTFIT or the Emboss program “Needle”. This software use the Needleman and Wunsch global alignment algorithm for aligning two sequences, over their entire length, maximizing the number of matches and minimizing the number of gaps. Generally, the default parameters are used, with a gap creation penalty=10 and gap extension penalty=0.5 (both for nucleotide and protein alignments). For nucleotides the default scoring matrix used is DNAFULL and for proteins the default scoring matrix is Blosum62 (Henikoff & Henikoff, 1992, PNAS 89, 10915-10919). Sequence alignments and scores for percentage sequence identity may for example be determined using software, such as EMBOSS, accessible at world wide web site of the EBI (ebi.ac.uk/Tools/emboss/). Alternatively, sequence similarity or identity may be determined by searching against databases (e.g. EMBL, GenBank) by using commonly known algorithms and output formats such as FASTA, BLAST, etc., but preferably hits should be retrieved and aligned pairwise to finally determine sequence identity.

If sequences to be compared with one another are of different length, the identity is to be determined by determining the identity in percent of the number of amino acids or nucleotides, respectively, which the shorter sequence shares with the longer sequence. Preferably, the identity is determined using the known and publicly available computer program ClustalW (Thompson et al., Nucleic Acids Research 22 (1994), 4673-4680). ClustalW is made publicly available by Julie Thompson (Thompson@EMBL-Heidelberg.DE) and Toby Gibson (Gibson@EMBL-Heidelberg.DE), European Molecular Biology Laboratory, Meyerhofstrasse 1, D 69117 Heidelberg, Germany. ClustalW can also be downloaded from various Internet pages, inter alia from IGBMC (Institut de Génétique et de Biologie Moléculaire et Cellulaire, B.P.163, 67404 Illkirch Cedex, France; ftp://ftp-igbmc.u-strasbg.fr/pub/) and from EBI (ftp://ftp.ebi.ac.uk/pub/software/) and all mirrored Internet pages of the EBI (European Bioinformatics Institute, Wellcome Trust Genome Campus, Hinxton, Cambridge CB10 1SD, UK).

Preferably, use is made of the ClustalW computer program of version 1.8 to determine the identity between proteins described in the context of the present invention and other proteins. Here, the parameters have to be set as follows: KTUPLE=1, TOPDIAG=5, WINDOW=5, PAIRGAP=3, GAPOPEN=10, GAPEXTEND=0.05, GAPDIST=8, MAXDIV=40, MATRIX=GONNET, ENDGAPS(OFF), NOPGAP, NOHGAP.

Preferably, use is made of the ClustalW computer program of version 1.8 to determine the identity for example between the nucleotide sequence of the nucleic acid molecules described in the context of the present invention and the nucleotide sequence of other nucleic acid molecules. Here, the parameters have to be set as follows:
KTUPLE=2, TOPDIAGS=4, PAIRGAP=5, DNAMATRIX:IUB, GAPOPEN=10, GAPEXT=5, MAXDIV=40, TRANSITIONS: unweighted.

“Identity” furthermore means that there is a functional and/or structural equivalence between the nucleic acid molecules in question or the proteins encoded by them. Functional equivalence means that the nucleic acid molecule sequences or the amino acid sequences encode a protein having the activity of a lipase. The nucleic acid molecules which are homologous to the molecules described above and represent derivatives of these molecules are generally variants of these molecules which represent modifications having the same biological function or catalyzing the same reaction, i.e. coding for a protein having the activity of a lipase. They may be either naturally occurring variants, for example sequences from other species, or mutations, where these mutations may have occurred in a natural manner or were introduced by targeted mutagenesis. Furthermore, the variants may be synthetically produced sequences. The allelic variants may be either naturally occurring variants or synthetically produced variants or variants generated by recombinant DNA techniques. However, concerning the present invention it is decisive that those variants encode proteins having lipase-activity and comprise the amino acid substitutions (replacements), deletions or insertions described herein concerning the proteins according to the invention.

A special type of derivatives are, for example, nucleic acid molecules which differ from the nucleic acid molecules described in the context of the present invention as a result of the degeneracy of the genetic code.

According to the NC-IUBMB (Nomenclature Committee of the International Union of Biochemistry and Molecular Biology) lipases belong to the class of hydrolases (EC 3). Hydrolase is a class of enzymes that commonly perform as biochemical catalysts that use water to break a chemical bond, which typically results in dividing a larger molecule into smaller molecules. The group of hydrolases comprises enzymes acting on ester bonds (EC 3.1) encompassing carboxylic ester hydrolases (EC 3.1.1) and as a subgroup lipases (EC 3.1.1.3). Lipases have been identified from plants, mammals and microorganisms including e.g. Pseudomonas, Vibrio, Acinetobacter, Burkholderia, Chromobacterium, cutinase from Fusarium solani (FSC), Candida antarctica A (CalA), Rhizopus oryzae (ROL), Thermomyces lanuginosus (TLL), Rhizomucor miehei (RML), Aspergillus Niger, Fusarium heterosporum, Fusarium oxysporum or Fusarium culmorum.

If a protein has the activity of a lipase, this can be detected with methods known and described in the art.

It is not decisive which method is used for detecting if a protein according to the invention has the activity of a lipase. Preferably, in connection with the present invention, the method is described in the “Example”-section.

Lipase variant proteins according to the invention may exhibit further amino acid modifications (amino acid substitutions, deletions or insertions) compared to the amino acid sequences described herein above in respect to the amino acid sequence shown under SEQ ID No. 1.

In addition to the lipase variants described herein above under item a) with reference to the amino acid sequence shown under SEQ ID No. 1, can have at least one, two, three, four, five, six or seven further amino acid substitution/s at position/s 79, 202, 280, 301, 3, 11, 17, 40 or 111. In other words, the inventive protein having the activity of a lipase is selected from the group consisting of a) proteins comprising the amino acid sequence shown in SEQ ID No. 1 apart from that the amino acid at position 186 is different from L and have at least one, two, three, four, five, six, seven or more further amino acid substitution selected from the group consisting of (i) the amino acid at position 79 is different from E; (ii) the amino acid at position 202 is different from A; (iii) the amino acid at position 280 is different from L; (iv) the amino acid at position 301 is different from D; (v) the amino acid at position 3 is different from M; (vi) the amino acid at position 11 is different from C; (vii) the amino acid at position 17 is different from L; (viii) the amino acid at position 40 is different from K; (ix) the amino acid at position 111 is different from Q; and b) proteins having an amino acid sequence having at least 80%, preferably 85%, more preferably 90%, further more preferably 95%, even more preferably 96%, even further more preferably 97%, particular preferably 98%, most preferably 99% identity with the amino acid sequence shown directly here above under a) given that the amino acid at position 186 is different from L and have at least one further amino acid substitution selected from the group (i) to (ix) mentioned directly here above. Preferably, the amino acid at position 79 is S, W or I, more preferably S; preferably the amino acid at position 202 is N; preferably the amino acid at position 280 is A; preferably the amino acid at position 301 is A; preferably the amino acid at position 3 is Q; preferably the amino acid at position 11 is A; preferably the amino acid at position 17 is P; preferably the amino acid at position 40 is M; preferably the amino acid at position 111 is E.

In addition, the lipase variant proteins according to the invention may exhibit, apart from the further amino acid modifications, additional amino acid substitutions compared to the amino acid sequences described herein above in respect to the amino acid sequence shown under SEQ ID No. 1. These additional amino acid substitutions relate to positions of the amino acid sequence that are different than the position/s 79, 202, 280, 301, 3, 11, 17, 40 or 111 which relate to the further amino acid modifications. The lipase variants described herein above under item a) with reference to the amino acid sequence shown under SEQ ID No. 1, can have at least one, two, three, four, five, six, seven or more additional amino acid substitution/s at position/s 4, 8, 18, 29, 31, 42, 84, 85, 192, 217, 309 or 312. The amino acid at position 4 is different from S, preferably the amino acid at that position is P. The amino acid at position 8 is different from K, preferably the amino acid at that position is E. The amino acid at position 18 is different from V, preferably the amino acid at that position is C. The amino acid at position 29 is different from N, preferably the amino acid at that position is W or H. The amino acid at position 31 is different from Q, preferably the amino acid at that position is W. The amino acid at position 42 is different from L, preferably the amino acid at that position is D. The amino acid at position 84 is different from N, preferably the amino acid at that position is T. The amino acid at position 85 is different from W, preferably the amino acid at that position is H. The amino acid at position 192 is different from F, preferably the amino acid at that position is A or V. The amino acid at position 217 is different from Q, preferably the amino acid at that position is M. The amino acid at position 309 is different from P, preferably the amino acid at that position is C. The amino acid at position 312 is different from P, preferably the amino acid at that position is N.

A further embodiment of the invention therefore concerns proteins according to the invention comprising further amino acid modifications, preferably those embodiments are proteins having the activity of a lipase, wherein the proteins are selected from the group consisting of
proteins comprising the amino acid sequence shown in SEQ ID No. 1 apart from that
   the amino acid at position 186 is different from L and the amino acid at position 79 is different from E, wherein it is preferred that the amino acid at position 186 is preferably W or Y, more preferably Y, and the amino acid at position 79 is preferably S, W or I, more preferably S;
   the amino acid at position 186 is different from L and the amino acid at position 202 is different from A, wherein it is preferred that the amino acid at position 186 is preferably W or Y, and the amino acid at position 202 is preferably N;
   the amino acid at position 186 is different from L and the amino acid at position 280 is different from L, wherein it is preferred that the amino acid at position 186 is preferably W or Y, and the amino acid at position 280 is preferably A;
   the amino acid at position 186 is different from L and the amino acid at position 301 is different from D, wherein it is preferred that the amino acid at position 186 is preferably W or Y, and the amino acid at position 301 is preferably A;
   the amino acid at position 186 is different from L and the amino acid at position 3 is different from M, wherein it is preferred that the amino acid at position 186 is preferably W or Y, and the amino acid at position 3 is preferably Q;
   the amino acid at position 186 is different from L and the amino acid at position 11 is different from C, wherein it is preferred that the amino acid at position 186 is preferably W or Y, and the amino acid at position 11 is preferably A;
   the amino acid at position 186 is different from L and the amino acid at position 17 is different from L, wherein it is preferred that the amino acid at position 186 is preferably W or Y, and the amino acid at position 17 is preferably P;
   the amino acid at position 186 is different from L and the amino acid at position 40 is different from K, wherein it is preferred that the amino acid at position 186 is preferably W or Y, and the amino acid at position 40 is preferably M;
   the amino acid at position 186 is different from L and the amino acid at position 111 is different from Q, wherein it is preferred that the amino acid at position 186 is preferably W or Y, and the amino acid at position 111 is preferably E;
   proteins having an amino acid sequence being at least 80%, preferably 85%, more preferably 90%, further more preferably 95%, even more preferably 96%, even further more preferably 97%, particular preferably 98%, most preferably 99% identity with the amino acid sequence shown under a) given that the amino acid at position 186 is different from L, wherein it is preferred that the amino acid at position 186 is preferably W or Y, and the proteins have at least one further amino acid substitution selected from the group shown under the listed punctuation marks directly here above.

Preferably, the lipase variants described herein above under item a) with reference to the amino acid sequence shown under SEQ ID No. 1, can have at least two further amino acid substitution/s at position/s 79, 202, 280, 301, 3, 11, 17, 40 or 111. Preferably, the amino acid at position 79 is S, W or I, more preferably S; preferably the amino acid at position 202 is N; preferably the amino acid at position 280 is A; preferably the amino acid at position 301 is A; preferably the amino acid at position 3 is Q; preferably the amino acid at position 11 is A; preferably the amino acid at position 17 is P; preferably the amino acid at position 40 is M; preferably the amino acid at position 111 is E.

A further embodiment of the invention therefore concerns proteins according to the invention comprising further amino acid modifications, preferably those embodiments are proteins having the activity of a lipase, wherein the proteins are selected from the group consisting of proteins comprising the amino acid sequence shown in SEQ ID NO. 1 apart from that
the amino acid at position 186 is different from L and the amino acid at position 79 is different from E and the amino acid at position 202 is different from A, wherein it is preferred that the amino acid at position 186 is preferably W or Y, and the amino acid at position 79 is preferably S, W or I, more preferably S, and the amino acid at position 202 is preferably N;
the amino acid at position 186 is different from L and the amino acid at position 79 is different from E and the amino acid at position 280 is different from L, wherein it is preferred that the amino acid at position 186 is preferably W or Y, and the amino acid at position 79 is preferably S, W or I, more preferably S, and the amino acid at position 280 is preferably A;
the amino acid at position 186 is different from L and the amino acid at position 79 is different from E and the amino acid at position 301 is different from D, wherein it is preferred that the amino acid at position 186 is preferably W or Y, and the amino acid at position 79 is preferably S, W or I, more preferably S, and the amino acid at position 301 is preferably A;
the amino acid at position 186 is different from L and the amino acid at position 79 is different from E and the amino acid at position 3 is different from M, wherein it is preferred that the amino acid at position 186 is preferably W or Y, and the amino acid at position 79 is preferably S, W or I, more preferably S, and the amino acid at position 3 is preferably Q;
the amino acid at position 186 is different from L and the amino acid at position 79 is different from E and the amino acid at position 11 is different from C, wherein it is preferred that the amino acid at position 186 is preferably W or Y, and the amino acid at position 79 is preferably S, W or I, more preferably S, and the amino acid at position 11 is preferably A;
the amino acid at position 186 is different from L and the amino acid at position 79 is different from E and the amino acid at position 17 is different from L, wherein it is preferred that the amino acid at position 186 is preferably W or Y, and the amino acid at position 79 is preferably S, W or I, more preferably S, and the amino acid at position 17 is preferably P;
the amino acid at position 186 is different from L and the amino acid at position 79 is different from E and the amino acid at position 40 is different from K, wherein it is preferred that the amino acid at position 186 is preferably W or Y, and the amino acid at position 79 is preferably S, W or I, more preferably S, and the amino acid at position 40 is preferably M;
the amino acid at position 186 is different from L and the amino acid at position 79 is different from E and the amino acid at position 111 is different from Q, wherein it is preferred that the amino acid at position 186 is preferably W or Y, and the amino acid at position 79 is preferably S, W or I, more preferably S, and the amino acid at position 111 is preferably E;
the amino acid at position 186 is different from L and the amino acid at position 202 is different from A and the amino acid at position 280 is different from L, wherein it is preferred that the amino acid at position 186 is preferably W or Y, and the amino acid at position 202 is preferably N and the amino acid at position 280 is preferably A;
the amino acid at position 186 is different from L and the amino acid at position 202 is different from A and the amino acid at position 301 is different from D, wherein it is preferred that the amino acid at position 186 is preferably W or Y, and the amino acid at position 202 is preferably N and the amino acid at position 301 is preferably A;
the amino acid at position 186 is different from L and the amino acid at position 202 is different from A and the amino acid at position 3 is different from M, wherein it is preferred that the amino acid at position 186 is preferably W or Y, and the amino acid at position 202 is preferably N and the amino acid at position 3 is preferably Q;
the amino acid at position 186 is different from L and the amino acid at position 202 is different from A and the amino acid at position 11 is different from C, wherein it is preferred that the amino acid at position 186 is preferably W or Y, and the amino acid at position 202 is preferably N and the amino acid at position 11 is preferably A;
the amino acid at position 186 is different from L and the amino acid at position 202 is different from A and the amino acid at position 17 is different from L, wherein it is preferred that the amino acid at position 186 is preferably W or Y, and the amino acid at position 202 is preferably N and the amino acid at position 17 is preferably P;
the amino acid at position 186 is different from L and the amino acid at position 202 is different from A and the amino acid at position 40 is different from K, wherein it is preferred that the amino acid at position 186 is preferably W or Y, and the amino acid at position 202 is preferably N and the amino acid at position 40 is preferably M;
the amino acid at position 186 is different from L and the amino acid at position 202 is different from A and the amino acid at position 111 is different from Q, wherein it is preferred that the amino acid at position 186 is preferably W or Y, and the amino acid at position 202 is preferably N and the amino acid at position 111 is preferably E;
the amino acid at position 186 is different from L and the amino acid at position 280 is different from L and the amino acid at position 301 is different from D, wherein it is preferred that the amino acid at position 186 is preferably W or Y, and the amino acid at position 280 is preferably A and the amino acid at position 301 is preferably A;
the amino acid at position 186 is different from L and the amino acid at position 280 is different from L and the amino acid at position 3 is different from M, wherein it is preferred that the amino acid at position 186 is preferably W or Y, and the amino acid at position 280 is preferably A and the amino acid at position 3 is preferably Q;
the amino acid at position 186 is different from L and the amino acid at position 280 is different from L and the amino acid at position 11 is different from C, wherein it is preferred that the amino acid at position 186 is preferably W or Y, and the amino acid at position 280 is preferably A and the amino acid at position 11 is preferably A;
the amino acid at position 186 is different from L and the amino acid at position 280 is different from L and the amino acid at position 17 is different from L, wherein it is preferred that the amino acid at position 186 is preferably W or Y, and the amino acid at position 280 is preferably A and the amino acid at position 17 is preferably P;
the amino acid at position 186 is different from L and the amino acid at position 280 is different from L and the amino acid at position 40 is different from K, wherein it is preferred that the amino acid at position 186 is preferably W or Y, and the amino acid at position 280 is preferably A and the amino acid at position 40 is preferably M;
the amino acid at position 186 is different from L and the amino acid at position 280 is different from L and the amino acid at position 111 is different from Q, wherein it is preferred that the amino acid at position 186 is preferably W or Y, and the amino acid at position 280 is preferably A and the amino acid at position 111 is preferably E;
the amino acid at position 186 is different from L and the amino acid at position 301 is different from D and the amino acid at position 3 is different from M, wherein it is preferred that the amino acid at position 186 is preferably W or Y, and the amino acid at position 301 is preferably A and the amino acid at position 3 is preferably Q;
the amino acid at position 186 is different from L and the amino acid at position 301 is different from D and the amino acid at position 11 is different from C, wherein it is preferred that the amino acid at position 186 is preferably W or Y, and the amino acid at position 301 is preferably A and the amino acid at position 11 is preferably A;
the amino acid at position 186 is different from L and the amino acid at position 301 is different from D and the amino acid at position 17 is different from L, wherein it is preferred that the amino acid at position 186 is preferably W or Y, and the amino acid at position 301 is preferably A and the amino acid at position 17 is preferably P;
the amino acid at position 186 is different from L and the amino acid at position 301 is different from D and the amino acid at position 40 is different from K, wherein it is preferred that the amino acid at position 186 is preferably W or Y, and the amino acid at position 301 is preferably A and the amino acid at position 40 is preferably M;
the amino acid at position 186 is different from L and the amino acid at position 301 is different from D and the amino acid at position 111 is different from Q, wherein it is preferred that the amino acid at position 186 is preferably W or Y, and the amino acid at position 301 is preferably A and the amino acid at position 111 is preferably E;
the amino acid at position 186 is different from L and the amino acid at position 3 is different from M and the amino acid at position 11 is different from C, wherein it is preferred that the amino acid at position 186 is preferably W or Y, and the amino acid at position 3 is preferably Q and the amino acid at position 11 is preferably A;
the amino acid at position 186 is different from L and the amino acid at position 3 is different from M and the amino acid at position 17 is different from L, wherein it is preferred that the amino acid at position 186 is preferably W or Y, and the amino acid at position 3 is preferably Q and the amino acid at position 17 is preferably P;
the amino acid at position 186 is different from L and the amino acid at position 3 is different from M and the amino acid at position 40 is different from K, wherein it is preferred that the amino acid at position 186 is preferably W or Y, and the amino acid at position 3 is preferably Q and the amino acid at position 40 is preferably M;
the amino acid at position 186 is different from L and the amino acid at position 3 is different from M and the amino acid at position 111 is different from Q, wherein it is preferred that the amino acid at position 186 is preferably W or Y, and the amino acid at position 3 is preferably Q and the amino acid at position 111 is preferably E;
the amino acid at position 186 is different from L and the amino acid at position 11 is different from C and the amino acid at position 17 is different from L, wherein it is preferred that the amino acid at position 186 is preferably W or Y, and the amino acid at position 11 is preferably A and the amino acid at position 17 is preferably P;
the amino acid at position 186 is different from L and the amino acid at position 11 is different from C and the amino acid at position 40 is different from K, wherein it is preferred that the amino acid at position 186 is preferably W or Y, and the amino acid at position 11 is preferably A and the amino acid at position 40 is preferably M;
the amino acid at position 186 is different from L and the amino acid at position 11 is different from C and the amino acid at position 111 is different from Q, wherein it is preferred that the amino acid at position 186 is preferably W or Y, and the amino acid at position 11 is preferably A and the amino acid at position 111 is preferably E;
the amino acid at position 186 is different from L and the amino acid at position 17 is different from L and the amino acid at position 40 is different from K, wherein it is preferred that the amino acid at position 186 is preferably W or Y, and the amino acid at position 17 is preferably P and the amino acid at position 40 is preferably M;
the amino acid at position 186 is different from L and the amino acid at position 17 is different from L and the amino acid at position 111 is different from Q, wherein it is preferred that the amino acid at position 186 is preferably W or Y, and the amino acid at position 17 is preferably P and the amino acid at position 111 is preferably E;
the amino acid at position 186 is different from L and the amino acid at position 40 is different from K and the amino acid at position 111 is different from Q, wherein it is preferred that the amino acid at position 186 is preferably W or Y, and the amino acid at position 40 is preferably M and the amino acid at position 111 is preferably E;
proteins having an amino acid sequence being at least 80%, preferably 85%, more preferably 90%, further more preferably 95%, even more preferably 96%, even further more preferably 97%, particular preferably 98%, most preferably 99% identity with the amino acid sequence shown under a) given that the amino acid at position 186 is different from L, wherein it is preferred that the amino acid at position 186 is preferably W or Y, and the proteins have at least two further amino acid substitutions selected from the group shown under the listed punctuation marks directly here above.

Preferably, the lipase variants described herein above under item a) with reference to the amino acid sequence shown under SEQ ID No. 1, can have at least three further amino acid substitution/s at position/s 79, 202, 280, 301, 3, 11, 17, 40 or 111. Preferably, the amino acid at position 79 is S, W or I, more preferably S; preferably the amino acid at position 202 is N; preferably the amino acid at position 280 is A; preferably the amino acid at position 301 is A; preferably the amino acid at position 3 is Q; preferably the amino acid at position 11 is A; preferably the amino acid at position 17 is P; preferably the amino acid at position 40 is M; preferably the amino acid at position 111 is E.

A further embodiment of the invention therefore concerns proteins according to the invention comprising further amino acid modifications, preferably those embodiments are proteins having the activity of a lipase, wherein the proteins are selected from the group consisting of proteins comprising the amino acid sequence shown in SEQ ID No. 1 apart from that
the amino acid at position 186 is different from L and the amino acid at position 79 is different from E and the amino acid at position 202 is different from A and the amino acid at position 280 is different from L, wherein it is preferred that the amino acid at position 186 is preferably W or Y, and the amino acid at position 79 is preferably S, W or I, more preferably S, and the amino acid at position 202 is preferably N and the amino acid at position 280 is preferably A;
the amino acid at position 186 is different from L and the amino acid at position 79 is different from E and the amino acid at position 202 is different from A and the amino acid at position 301 is different from D, wherein it is preferred that the amino acid at position 186 is preferably W or Y, and the amino acid at position 79 is preferably S, W or I, more preferably S, and the amino acid at position 202 is preferably N and the amino acid at position 301 is preferably A;
the amino acid at position 186 is different from L and the amino acid at position 79 is different from E and the amino acid at position 202 is different from A and the amino acid at position 3 is different from M, wherein it is preferred that the amino acid at position 186 is preferably W or Y, and the amino acid at position 79 is preferably S, W or I, more preferably S, and the amino acid at position 202 is preferably N and the amino acid at position 3 is preferably Q;
the amino acid at position 186 is different from L and the amino acid at position 79 is different from E and the amino acid at position 202 is different from A and the amino acid at position 11 is different from C, wherein it is preferred that the amino acid at position 186 is preferably W or Y, and the amino acid at position 79 is preferably S, W or I, more preferably S, and the amino acid at position 202 is preferably N and the amino acid at position 11 is preferably A;
the amino acid at position 186 is different from L and the amino acid at position 79 is different from E and the amino acid at position 202 is different from A and the amino acid at position 17 is different from L, wherein it is preferred that the amino acid at position 186 is preferably W or Y, and the amino acid at position 79 is preferably S, W or I, more preferably S, and the amino acid at position 202 is preferably N and the amino acid at position 17 is preferably P;
the amino acid at position 186 is different from L and the amino acid at position 79 is different from E and the amino acid at position 202 is different from A and the amino acid at position 40 is different from K, wherein it is preferred that the amino acid at position 186 is preferably W or Y, and the amino acid at position 79 is preferably S, W or I, more preferably S, and the amino acid at position 202 is preferably N and the amino acid at position 40 is preferably M;
the amino acid at position 186 is different from L and the amino acid at position 79 is different from E and the amino acid at position 202 is different from A and the amino acid at position 111 is different from Q, wherein it is preferred that the amino acid at position 186 is preferably W or Y, and the amino acid at position 79 is preferably S, W or I, more preferably S, and the amino acid at position 202 is preferably N and the amino acid at position 111 is preferably E;
the amino acid at position 186 is different from L and the amino acid at position 79 is different from E and the amino acid at position 280 is different from L and the amino acid at position 301 is different from D, wherein it is preferred that the amino acid at position 186 is preferably W or Y, and the amino acid at position 79 is preferably S, W or I, more preferably S, and the amino acid at position 280 is preferably A and the amino acid at position 301 is preferably A;
the amino acid at position 186 is different from L and the amino acid at position 79 is different from E and the amino acid at position 280 is different from L and the amino acid at position 3 is different from M, wherein it is preferred that the amino acid at position 186 is preferably W or Y, and the amino acid at position 79 is preferably S, W or I, more preferably S, and the amino acid at position 280 is preferably A and the amino acid at position 3 is preferably Q;
the amino acid at position 186 is different from L and the amino acid at position 79 is different from E and the amino acid at position 280 is different from L and the amino acid at position 11 is different from C, wherein it is preferred that the amino acid at position 186 is preferably W or Y, and the amino acid at position 79 is preferably S, W or I, more preferably S, and the amino acid at position 280 is preferably A and the amino acid at position 11 is preferably A;
the amino acid at position 186 is different from L and the amino acid at position 79 is different from E and the amino acid at position 280 is different from L and the amino acid at position 17 is different from L, wherein it is preferred that the amino acid at position 186 is preferably W or Y, and the amino acid at position 79 is preferably S, W or I, more preferably S, and the amino acid at position 280 is preferably A and the amino acid at position 17 is preferably P;
the amino acid at position 186 is different from L and the amino acid at position 79 is different from E and the amino acid at position 280 is different from L and the amino acid at position 40 is different from K, wherein it is preferred that the amino acid at position 186 is preferably W or Y, and the amino acid at position 79 is preferably S, W or I, more preferably S, and the amino acid at position 280 is preferably A and the amino acid at position 40 is preferably M;
the amino acid at position 186 is different from L and the amino acid at position 79 is different from E and the amino acid at position 280 is different from L and the amino acid at position 111 is different from Q, wherein it is preferred that the amino acid at position 186 is preferably W or Y, and the amino acid at position 79 is preferably S, W or I, more preferably S, and the amino acid at position 280 is preferably A and the amino acid at position 111 is preferably E;
the amino acid at position 186 is different from L and the amino acid at position 79 is different from E and the amino acid at position 301 is different from D and the amino acid at position 3 is different from M, wherein it is preferred that the amino acid at position 186 is preferably W or Y, and the amino acid at position 79 is preferably S, W or I, more preferably S, and the amino acid at position 301 is preferably A and the amino acid at position 3 is preferably Q;
the amino acid at position 186 is different from L and the amino acid at position 79 is different from E and the amino acid at position 301 is different from D and the amino acid at position 11 is different from C, wherein it is preferred that the amino acid at position 186 is preferably W or Y, and the amino acid at position 79 is preferably S, W or I, more preferably S, and the amino acid at position 301 is preferably A and the amino acid at position 11 is preferably A;
the amino acid at position 186 is different from L and the amino acid at position 79 is different from E and the amino acid at position 301 is different from D and the amino acid at position 17 is different from L, wherein it is preferred that the amino acid at position 186 is preferably W or Y, and the amino acid at position 79 is preferably S, W or I, more preferably S, and the amino acid at position 301 is preferably A and the amino acid at position 17 is preferably P;
the amino acid at position 186 is different from L and the amino acid at position 79 is different from E and the amino acid at position 301 is different from D and the amino acid at position 40 is different from K, wherein it is preferred that the amino acid at position 186 is preferably W or Y, more preferably Y, and the amino acid at position 79 is preferably S, W or I, more preferably S, and the amino acid at position 301 is preferably A and the amino acid at position 40 is preferably M, wherein it is especially preferred that the amino acid at position 186 is Y, and the amino acid at position 79 is S, and the amino acid at position 301 is A and the amino acid at position 40 is M;
the amino acid at position 186 is different from L and the amino acid at position 79 is different from E and the amino acid at position 301 is different from D and the amino acid at position 111 is different from Q, wherein it is preferred that the amino acid at position 186 is preferably W or Y, and the amino acid at position 79 is preferably S, W or I, more preferably S, and the amino acid at position 301 is preferably A and the amino acid at position 111 is preferably E;
the amino acid at position 186 is different from L and the amino acid at position 79 is different from E and the amino acid at position 3 is different from M and the amino acid at position 11 is different from C, wherein it is preferred that the amino acid at position 186 is preferably W or Y, and the amino acid at position 79 is preferably S, W or I, more preferably S, and the amino acid at position 3 is preferably Q and the amino acid at position 11 is preferably A;
the amino acid at position 186 is different from L and the amino acid at position 79 is different from E and the amino acid at position 3 is different from M and the amino acid at position 17 is different from L, wherein it is preferred that the amino acid at position 186 is preferably W or Y, and the amino acid at position 79 is preferably S, W or I, more preferably S, and the amino acid at position 3 is preferably Q and the amino acid at position 17 is preferably P;
the amino acid at position 186 is different from L and the amino acid at position 79 is different from E and the amino acid at position 3 is different from M and the amino acid at position 40 is different from K, wherein it is preferred that the amino acid at position 186 is preferably W or Y, and the amino acid at position 79 is preferably S, W or I, more preferably S, and the amino acid at position 3 is preferably Q and the amino acid at position 40 is preferably M;
the amino acid at position 186 is different from L and the amino acid at position 79 is different from E and the amino acid at position 3 is different from M and the amino acid at position 111 is different from Q, wherein it is preferred that the amino acid at position 186 is preferably W or Y, and the amino acid at position 79 is preferably S, W or I, more preferably S, and the amino acid at position 3 is preferably Q and the amino acid at position 111 is preferably E;
the amino acid at position 186 is different from L and the amino acid at position 79 is different from E and the amino acid at position 11 is different from C and the amino acid at position 17 is different from L, wherein it is preferred that the amino acid at position 186 is preferably W or Y, and the amino acid at position 79 is preferably S, W or I, more preferably S, and the amino acid at position 11 is preferably A and the amino acid at position 17 is preferably P;
the amino acid at position 186 is different from L and the amino acid at position 79 is different from E and the amino acid at position 11 is different from C and the amino acid at position 40 is different from K, wherein it is preferred that the amino acid at position 186 is preferably W or Y, and the amino acid at position 79 is preferably S, W or I, more preferably S, and the amino acid at position 11 is preferably A and the amino acid at position 40 is preferably M;
the amino acid at position 186 is different from L and the amino acid at position 79 is different from E and the amino acid at position 11 is different from C and the amino acid at position 111 is different from Q, wherein it is preferred that the amino acid at position 186 is preferably W or Y, and the amino acid at position 79 is preferably S, W or I, more preferably S, and the amino acid at position 11 is preferably A and the amino acid at position 111 is preferably E;
the amino acid at position 186 is different from L and the amino acid at position 79 is different from E and the amino acid at position 17 is different from L and the amino acid at position 40 is different from K, wherein it is preferred that the amino acid at position 186 is preferably W or Y, and the amino acid at position 79 is preferably S, W or I, more preferably S, and the amino acid at position 17 is preferably P and the amino acid at position 40 is preferably M;
the amino acid at position 186 is different from L and the amino acid at position 79 is different from E and the amino acid at position 17 is different from L and the amino acid at position 111 is different from Q, wherein it is preferred that the amino acid at position 186 is preferably W or Y, and the amino acid at position 79 is preferably S, W or I, more preferably S, and the amino acid at position 17 is preferably P and the amino acid at position 111 is preferably E;
the amino acid at position 186 is different from L and the amino acid at position 79 is different from E and the amino acid at position 40 is different from K and the amino acid at position 111 is different from Q, wherein it is preferred that the amino acid at position 186 is preferably W or Y, and the amino acid at position 79 is preferably S, W or I, more preferably S, and the amino acid at position 40 is preferably M and the amino acid at position 111 is preferably E;
the amino acid at position 186 is different from L and the amino acid at position 202 is different from A and the amino acid at position 280 is different from L and the amino acid at position 301 is different from D, wherein it is preferred that the amino acid at position 186 is preferably W or Y, and the amino acid at position 202 is preferably N and the amino acid at position 280 is preferably A and the amino acid at position 301 is preferably A;
the amino acid at position 186 is different from L and the amino acid at position 202 is different from A and the amino acid at position 280 is different from L and the amino acid at position 3 is different from M, wherein it is preferred that the amino acid at position 186 is preferably W or Y, and the amino acid at position 202 is preferably N and the amino acid at position 280 is preferably A and the amino acid at position 3 is preferably Q;
the amino acid at position 186 is different from L and the amino acid at position 202 is different from A and the amino acid at position 280 is different from L and the amino acid at position 11 is different from C, wherein it is preferred that the amino acid at position 186 is preferably W or Y, and the amino acid at position 202 is preferably N and the amino acid at position 280 is preferably A and the amino acid at position 11 is preferably A;
the amino acid at position 186 is different from L and the amino acid at position 202 is different from A and the amino acid at position 280 is different from L and the amino acid at position 17 is different from L, wherein it is preferred that the amino acid at position 186 is preferably W or Y, and the amino acid at position 202 is preferably N and the amino acid at position 280 is preferably A and the amino acid at position 17 is preferably P;
the amino acid at position 186 is different from L and the amino acid at position 202 is different from A and the amino acid at position 280 is different from L and the amino acid at position 40 is different from K, wherein it is preferred that the amino acid at position 186 is preferably W or Y, and the amino acid at position 202 is preferably N and the amino acid at position 280 is preferably A and the amino acid at position 40 is preferably M;
the amino acid at position 186 is different from L and the amino acid at position 202 is different from A and the amino acid at position 280 is different from L and the amino acid at position 111 is different from Q, wherein it is preferred that the amino acid at position 186 is preferably W or Y, and the amino acid at position 202 is preferably N and the amino acid at position 280 is preferably A and the amino acid at position 111 is preferably E;
the amino acid at position 186 is different from L and the amino acid at position 202 is different from A and the amino acid at position 301 is different from D and the amino acid at position 3 is different from M, wherein it is preferred that the amino acid at position 186 is preferably W or Y, and the amino acid at position 202 is preferably N and the amino acid at position 301 is preferably A and the amino acid at position 3 is preferably Q;
the amino acid at position 186 is different from L and the amino acid at position 202 is different from A and the amino acid at position 301 is different from D and the amino acid at position 11 is different from C, wherein it is preferred that the amino acid at position 186 is preferably W or Y, and the amino acid at position 202 is preferably N and the amino acid at position 301 is preferably A and the amino acid at position 11 is preferably A;
the amino acid at position 186 is different from L and the amino acid at position 202 is different from A and the amino acid at position 301 is different from D and the amino acid at position 17 is different from L, wherein it is preferred that the amino acid at position 186 is preferably W or Y, and the amino acid at position 202 is preferably N and the amino acid at position 301 is preferably A and the amino acid at position 17 is preferably P;
the amino acid at position 186 is different from L and the amino acid at position 202 is different from A and the amino acid at position 301 is different from D and the amino acid at position 40 is different from K, wherein it is preferred that the amino acid at position 186 is preferably W or Y, and the amino acid at position 202 is preferably N and the amino acid at position 301 is preferably A and the amino acid at position 40 is preferably M;
the amino acid at position 186 is different from L and the amino acid at position 202 is different from A and the amino acid at position 301 is different from D and the amino acid at position 111 is different from Q, wherein it is preferred that the amino acid at position 186 is preferably W or Y, and the amino acid at position 202 is preferably N and the amino acid at position 301 is preferably A and the amino acid at position 111 is preferably E;
the amino acid at position 186 is different from L and the amino acid at position 202 is different from A and the amino acid at position 3 is different from M and the amino acid at position 11 is different from C, wherein it is preferred that the amino acid at position 186 is preferably W or Y, and the amino acid at position 202 is preferably N and the amino acid at position 3 is preferably Q and the amino acid at position 11 is preferably A;
the amino acid at position 186 is different from L and the amino acid at position 202 is different from A and the amino acid at position 3 is different from M and the amino acid at position 17 is different from L, wherein it is preferred that the amino acid at position 186 is preferably W or Y, and the amino acid at position 202 is preferably N and the amino acid at position 3 is preferably Q and the amino acid at position 17 is preferably P;
the amino acid at position 186 is different from L and the amino acid at position 202 is different from A and the amino acid at position 3 is different from M and the amino acid at position 40 is different from K, wherein it is preferred that the amino acid at position 186 is preferably W or Y, and the amino acid at position 202 is preferably N and the amino acid at position 3 is preferably Q and the amino acid at position 40 is preferably M;
the amino acid at position 186 is different from L and the amino acid at position 202 is different from A and the amino acid at position 3 is different from M and the amino acid at position 111 is different from Q, wherein it is preferred that the amino acid at position 186 is preferably W or Y, and the amino acid at position 202 is preferably N and the amino acid at position 3 is preferably Q and the amino acid at position 111 is preferably E;
the amino acid at position 186 is different from L and the amino acid at position 202 is different from A and the amino acid at position 11 is different from C and the amino acid at position 17 is different from L, wherein it is preferred that the amino acid at position 186 is preferably W or Y, and the amino acid at position 202 is preferably N and the amino acid at position 11 is preferably A and the amino acid at position 17 is preferably P;
the amino acid at position 186 is different from L and the amino acid at position 202 is different from A and the amino acid at position 11 is different from C and the amino acid at position 40 is different from K, wherein it is preferred that the amino acid at position 186 is preferably W or Y, and the amino acid at position 202 is preferably N and the amino acid at position 11 is preferably A and the amino acid at position 40 is preferably M;
the amino acid at position 186 is different from L and the amino acid at position 202 is different from A and the amino acid at position 11 is different from C and the amino acid at position 111 is different from Q, wherein it is preferred that the amino acid at position 186 is preferably W or Y, and the amino acid at position 202 is preferably N and the amino acid at position 11 is preferably A and the amino acid at position 111 is preferably E;
the amino acid at position 186 is different from L and the amino acid at position 202 is different from A and the amino acid at position 17 is different from L and the amino acid at position 40 is different from K, wherein it is preferred that the amino acid at position 186 is preferably W or Y, and the amino acid at position 202 is preferably N and the amino acid at position 17 is preferably P and the amino acid at position 40 is preferably M;
the amino acid at position 186 is different from L and the amino acid at position 202 is different from A and the amino acid at position 17 is different from L and the amino acid at position 111 is different from Q, wherein it is preferred that the amino acid at position 186 is preferably W or Y, and the amino acid at position 202 is preferably N and the amino acid at position 17 is preferably P and the amino acid at position 111 is preferably E;
the amino acid at position 186 is different from L and the amino acid at position 202 is different from A and the amino acid at position 40 is different from K and the amino acid at position 111 is different from Q, wherein it is preferred that the amino acid at position 186 is preferably W or Y, and the amino acid at position 202 is preferably N and the amino acid at position 40 is preferably M and the amino acid at position 111 is preferably E;
the amino acid at position 186 is different from L and the amino acid at position 280 is different from L and the amino acid at position 301 is different from D and the amino acid at position 3 is different from M, wherein it is preferred that the amino acid at position 186 is preferably W or Y, and the amino acid at position 280 is preferably A and the amino acid at position 301 is preferably A and the amino acid at position 3 is preferably Q;
the amino acid at position 186 is different from L and the amino acid at position 280 is different from L and the amino acid at position 301 is different from D and the amino acid at position 11 is different from C, wherein it is preferred that the amino acid at position 186 is preferably W or Y, and the amino acid at position 280 is preferably A and the amino acid at position 301 is preferably A and the amino acid at position 11 is preferably A;
the amino acid at position 186 is different from L and the amino acid at position 280 is different from L and the amino acid at position 301 is different from D and the amino acid at position 17 is different from L, wherein it is preferred that the amino acid at position 186 is preferably W or Y, and the amino acid at position 280 is preferably A and the amino acid at position 301 is preferably A and the amino acid at position 17 is preferably P;
the amino acid at position 186 is different from L and the amino acid at position 280 is different from L and the amino acid at position 301 is different from D and the amino acid at position 40 is different from K, wherein it is preferred that the amino acid at position 186 is preferably W or Y, and the amino acid at position 280 is preferably A and the amino acid at position 301 is preferably A and the amino acid at position 40 is preferably M;
the amino acid at position 186 is different from L and the amino acid at position 280 is different from L and the amino acid at position 301 is different from D and the amino acid at position 111 is different from Q, wherein it is preferred that the amino acid at position 186 is preferably W or Y, and the amino acid at position 280 is preferably A and the amino acid at position 301 is preferably A and the amino acid at position 111 is preferably E;
the amino acid at position 186 is different from L and the amino acid at position 280 is different from L and the amino acid at position 3 is different from M and the amino acid at position 11 is different from C, wherein it is preferred that the amino acid at position 186 is preferably W or Y, and the amino acid at position 280 is preferably A and the amino acid at position 3 is preferably Q and the amino acid at position 11 is preferably A;
the amino acid at position 186 is different from L and the amino acid at position 280 is different from L and the amino acid at position 3 is different from M and the amino acid at position 17 is different from L, wherein it is preferred that the amino acid at position 186 is preferably W or Y, and the amino acid at position 280 is preferably A and the amino acid at position 3 is preferably Q and the amino acid at position 17 is preferably P;
the amino acid at position 186 is different from L and the amino acid at position 280 is different from L and the amino acid at position 3 is different from M and the amino acid at position 40 is different from K, wherein it is preferred that the amino acid at position 186 is preferably W or Y, and the amino acid at position 280 is preferably A and the amino acid at position 3 is preferably Q and the amino acid at position 40 is preferably M;
the amino acid at position 186 is different from L and the amino acid at position 280 is different from L and the amino acid at position 3 is different from M and the amino acid at position 111 is different from Q, wherein it is preferred that the amino acid at position 186 is preferably W or Y, and the amino acid at position 280 is preferably A and the amino acid at position 3 is preferably Q and the amino acid at position 111 is preferably E;
the amino acid at position 186 is different from L and the amino acid at position 280 is different from L and the amino acid at position 11 is different from C and the amino acid at position 17 is different from L, wherein it is preferred that the amino acid at position 186 is preferably W or Y, and the amino acid at position 280 is preferably A and the amino acid at position 11 is preferably A and the amino acid at position 17 is preferably P;
the amino acid at position 186 is different from L and the amino acid at position 280 is different from L and the amino acid at position 11 is different from C and the amino acid at position 40 is different from K, wherein it is preferred that the amino acid at position 186 is preferably W or Y, and the amino acid at position 280 is preferably A and the amino acid at position 11 is preferably A and the amino acid at position 40 is preferably M;
the amino acid at position 186 is different from L and the amino acid at position 280 is different from L and the amino acid at position 11 is different from C and the amino acid at position 111 is different from Q, wherein it is preferred that the amino acid at position 186 is preferably W or Y, and the amino acid at position 280 is preferably A and the amino acid at position 11 is preferably A and the amino acid at position 111 is preferably E;
the amino acid at position 186 is different from L and the amino acid at position 280 is different from L and the amino acid at position 17 is different from L and the amino acid at position 40 is different from K, wherein it is preferred that the amino acid at position 186 is preferably W or Y, and the amino acid at position 280 is preferably A and the amino acid at position 17 is preferably P and the amino acid at position 40 is preferably M;
the amino acid at position 186 is different from L and the amino acid at position 280 is different from L and the amino acid at position 17 is different from L and the amino acid at position 111 is different from Q, wherein it is preferred that the amino acid at position 186 is preferably W or Y, and the amino acid at position 280 is preferably A and the amino acid at position 17 is preferably P and the amino acid at position 111 is preferably E;
the amino acid at position 186 is different from L and the amino acid at position 280 is different from L and the amino acid at position 40 is different from K and the amino acid at position 111 is different from Q, wherein it is preferred that the amino acid at position 186 is preferably W or Y, and the amino acid at position 280 is preferably A and the amino acid at position 40 is preferably M and the amino acid at position 111 is preferably E;
the amino acid at position 186 is different from L and the amino acid at position 301 is different from D and the amino acid at position 3 is different from M and the amino acid at position 11 is different from C, wherein it is preferred that the amino acid at position 186 is preferably W or Y, and the amino acid at position 301 is preferably A and the amino acid at position 3 is preferably Q and the amino acid at position 11 is preferably A;
the amino acid at position 186 is different from L and the amino acid at position 301 is different from D and the amino acid at position 3 is different from M and the amino acid at position 17 is different from L, wherein it is preferred that the amino acid at position 186 is preferably W or Y, and the amino acid at position 301 is preferably A and the amino acid at position 3 is preferably Q and the amino acid at position 17 is preferably P;
the amino acid at position 186 is different from L and the amino acid at position 301 is different from D and the amino acid at position 3 is different from M and the amino acid at position 40 is different from K, wherein it is preferred that the amino acid at position 186 is preferably W or Y, and the amino acid at position 301 is preferably A and the amino acid at position 3 is preferably Q and the amino acid at position 40 is preferably M;
the amino acid at position 186 is different from L and the amino acid at position 301 is different from D and the amino acid at position 3 is different from M and the amino acid at position 111 is different from Q, wherein it is preferred that the amino acid at position 186 is preferably W or Y, and the amino acid at position 301 is preferably A and the amino acid at position 3 is preferably Q and the amino acid at position 111 is preferably E;
the amino acid at position 186 is different from L and the amino acid at position 301 is different from D and the amino acid at position 11 is different from C and the amino acid at position 17 is different from L, wherein it is preferred that the amino acid at position 186 is preferably W or Y, and the amino acid at position 301 is preferably A and the amino acid at position 11 is preferably A and the amino acid at position 17 is preferably P;
the amino acid at position 186 is different from L and the amino acid at position 301 is different from D and the amino acid at position 11 is different from C and the amino acid at position 40 is different from K, wherein it is preferred that the amino acid at position 186 is preferably W or Y, and the amino acid at position 301 is preferably A and the amino acid at position 11 is preferably A and the amino acid at position 40 is preferably M;
the amino acid at position 186 is different from L and the amino acid at position 301 is different from D and the amino acid at position 11 is different from C and the amino acid at position 111 is different from Q, wherein it is preferred that the amino acid at position 186 is preferably W or Y, and the amino acid at position 301 is preferably A and the amino acid at position 11 is preferably A and the amino acid at position 111 is preferably E;
the amino acid at position 186 is different from L and the amino acid at position 301 is different from D and the amino acid at position 17 is different from L and the amino acid at position 40 is different from K, wherein it is preferred that the amino acid at position 186 is preferably W or Y, and the amino acid at position 301 is preferably A and the amino acid at position 17 is preferably P and the amino acid at position 40 is preferably M;
the amino acid at position 186 is different from L and the amino acid at position 301 is different from D and the amino acid at position 17 is different from L and the amino acid at position 111 is different from Q, wherein it is preferred that the amino acid at position 186 is preferably W or Y, and the amino acid at position 301 is preferably A and the amino acid at position 17 is preferably P and the amino acid at position 111 is preferably E;
the amino acid at position 186 is different from L and the amino acid at position 301 is different from D and the amino acid at position 40 is different from K and the amino acid at position 111 is different from Q, wherein it is preferred that the amino acid at position 186 is preferably W or Y, and the amino acid at position 301 is preferably A and the amino acid at position 40 is preferably M and the amino acid at position 111 is preferably E;
the amino acid at position 186 is different from L and the amino acid at position 3 is different from M and the amino acid at position 11 is different from C and the amino acid at position 17 is different from L, wherein it is preferred that the amino acid at position 186 is preferably W or Y, and the amino acid at position 3 is preferably Q and the amino acid at position 11 is preferably A and the amino acid at position 17 is preferably P;
the amino acid at position 186 is different from L and the amino acid at position 3 is different from M and the amino acid at position 11 is different from C and the amino acid at position 40 is different from K, wherein it is preferred that the amino acid at position 186 is preferably W or Y, and the amino acid at position 3 is preferably Q and the amino acid at position 11 is preferably A and the amino acid at position 40 is preferably M;
the amino acid at position 186 is different from L and the amino acid at position 3 is different from M and the amino acid at position 11 is different from C and the amino acid at position 111 is different from Q, wherein it is preferred that the amino acid at position 186 is preferably W or Y, and the amino acid at position 3 is preferably Q and the amino acid at position 11 is preferably A and the amino acid at position 111 is preferably E;
the amino acid at position 186 is different from L and the amino acid at position 3 is different from M and the amino acid at position 17 is different from L and the amino acid at position 40 is different from K, wherein it is preferred that the amino acid at position 186 is preferably W or Y, and the amino acid at position 3 is preferably Q and the amino acid at position 17 is preferably P and the amino acid at position 40 is preferably M;
the amino acid at position 186 is different from L and the amino acid at position 3 is different from M and the amino acid at position 17 is different from L and the amino acid at position 111 is different from Q, wherein it is preferred that the amino acid at position 186 is preferably W or Y, and the amino acid at position 3 is preferably Q and the amino acid at position 17 is preferably P and the amino acid at position 111 is preferably E;
the amino acid at position 186 is different from L and the amino acid at position 3 is different from M and the amino acid at position 40 is different from K and the amino acid at position 111 is different from Q, wherein it is preferred that the amino acid at position 186 is preferably W or Y, and the amino acid at position 3 is preferably Q and the amino acid at position 40 is preferably M and the amino acid at position 111 is preferably E;
the amino acid at position 186 is different from L and the amino acid at position 11 is different from C and the amino acid at position 17 is different from L and the amino acid at position 40 is different from K, wherein it is preferred that the amino acid at position 186 is preferably W or Y, and the amino acid at position 11 is preferably A and the amino acid at position 17 is preferably P and the amino acid at position 40 is preferably M;
the amino acid at position 186 is different from L and the amino acid at position 11 is different from C and the amino acid at position 17 is different from L and the amino acid at position 111 is different from Q, wherein it is preferred that the amino acid at position 186 is preferably W or Y, and the amino acid at position 11 is preferably A and the amino acid at position 17 is preferably P and the amino acid at position 111 is preferably E;
the amino acid at position 186 is different from L and the amino acid at position 11 is different from C and the amino acid at position 40 is different from K and the amino acid at position 111 is different from Q, wherein it is preferred that the amino acid at position 186 is preferably W or Y, and the amino acid at position 11 is preferably A and the amino acid at position 40 is preferably M and the amino acid at position 111 is preferably E;
the amino acid at position 186 is different from L and the amino acid at position 17 is different from L and the amino acid at position 40 is different from K and the amino acid at position 111 is different from Q, wherein it is preferred that the amino acid at position 186 is preferably W or Y, and the amino acid at position 17 is preferably P and the amino acid at position 40 is preferably M and the amino acid at position 111 is preferably E;
proteins having an amino acid sequence being at least 80%, preferably 85%, more preferably 90%, further more preferably 95%, even more preferably 96%, even further more preferably 97%, particular preferably 98%, most preferably 99% identity with the amino acid sequence shown under a) given that the amino acid at position 186 is different from L wherein it is preferred that the amino acid at position 186 is preferably W or Y, more preferably Y, and the proteins have at least three further amino acid substitutions selected from the group shown under the listed punctuation marks directly here above.

The lipase variants described herein above under item a) with reference to the amino acid sequence shown under SEQ ID No. 1, can have at least four further amino acid substitution/s at position/s 79, 202, 280, 301, 3, 11, 17, 40 or 111. Preferably, the amino acid at position 79 is S, W or I, more preferably S; preferably the amino acid at position 202 is N; preferably the amino acid at position 280 is A; preferably the amino acid at position 301 is A; preferably the amino acid at position 3 is Q; preferably the amino acid at position 11 is A; preferably the amino acid at position 17 is P; preferably the amino acid at position 40 is M; preferably the amino acid at position 111 is E.

The lipase variants described herein above under item a) with reference to the amino acid sequence shown under SEQ ID No. 1, can have at least five further amino acid substitution/s at position/s 79, 202, 280, 301, 3, 11, 17, 40 or 111. Preferably, the amino acid at position 79 is S, W or I, more preferably S; preferably the amino acid at position 202 is N; preferably the amino acid at position 280 is A; preferably the amino acid at position 301 is A; preferably the amino acid at position 3 is Q; preferably the amino acid at position 11 is A; preferably the amino acid at position 17 is P; preferably the amino acid at position 40 is M; preferably the amino acid at position 111 is E.

The lipase variants described herein above under item a) with reference to the amino acid sequence shown under SEQ ID No. 1, can have at least six further amino acid substitution/s at position/s 79, 202, 280, 301, 3, 11, 17, 40 or 111. Preferably, the amino acid at position 79 is S, W or 1, more preferably S; preferably the amino acid at position 202 is N; preferably the amino acid at position 280 is A; preferably the amino acid at position 301 is A; preferably the amino acid at position 3 is Q; preferably the amino acid at position 11 is A; preferably the amino acid at position 17 is P; preferably the amino acid at position 40 is M; preferably the amino acid at position 111 is E.

The lipase variants described herein above under item a) with reference to the amino acid sequence shown under SEQ ID No. 1, can have at least seven further amino acid substitution/s at position/s 79, 202, 280, 301, 3, 11, 17, 40 or 111. Preferably, the amino acid at position 79 is S, W or I, more preferably S; preferably the amino acid at position 202 is N; preferably the amino acid at position 280 is A; preferably the amino acid at position 301 is A; preferably the amino acid at position 3 is Q; preferably the amino acid at position 11 is A; preferably the amino acid at position 17 is P; preferably the amino acid at position 40 is M; preferably the amino acid at position 111 is E.

Preferred embodiments of the invention are proteins according to the invention encoding lipases having the amino acid sequences shown under SEQ ID Nos. 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401.

Further proteins having the activity of a lipolytic enzyme or lipase respectively have been tested. The amino acid sequences of these further proteins represent variants of the amino acid sequence represented by the amino acid in SEQ ID No. 1, wherein:
in the amino acid sequence shown under SEQ ID No. 1 the two amino acids at positions 40 and 79 are different from the amino acids given at the respective amino acid positions in the sequence shown under SEQ ID No. 1. In a particular tested variant, with reference to the amino acid sequence shown under SEQ ID No. 1, the amino acid at position 40 is M and the amino acid at position 79 is S;
in the amino acid sequence shown under SEQ ID No. 1 the two amino acids at positions 40 and 186 are different from the amino acids given at the respective amino acid positions in the sequence shown under SEQ ID No. 1. In a particular tested variant, with reference to the amino acid sequence shown under SEQ ID No. 1, the amino acid at position 40 is M and the amino acid at position 186 is Y;
in the amino acid sequence shown under SEQ ID No. 1 the two amino acids at positions 40 and 301 are different from the amino acids given at the respective amino acid positions in the sequence shown under SEQ ID No. 1. In a particular tested variant, with reference to the amino acid sequence shown under SEQ ID No. 1, the amino acid at position 40 is M and the amino acid at position 301 is A;
in the amino acid sequence shown under SEQ ID No. 1 the two amino acids at positions 79 and 186 are different from the amino acids given at the respective amino acid positions in the sequence shown under SEQ ID No. 1. In a particular tested variant, with reference to the amino acid sequence shown under SEQ ID No. 1, the amino acid at position 79 is S and the amino acid at position 186 is Y;
in the amino acid sequence shown under SEQ ID No. 1 the two amino acids at positions 79 and 301 are different from the amino acids given at the respective amino acid positions in the sequence shown under SEQ ID No. 1. In a particular tested variant, with reference to the amino acid sequence shown under SEQ ID No. 1, the amino acid at position 79 is S and the amino acid at position 301 is A;
in the amino acid sequence shown under SEQ ID No. 1 the two amino acids at positions 186 and 301 are different from the amino acids given at the respective amino acid positions in the sequence shown under SEQ ID No. 1. In a particular tested variant, with reference to the amino acid sequence shown under SEQ ID No. 1, the amino acid at position 186 is Y and the amino acid at position 301 is A.

More further proteins having the activity of a lipolytic enzyme or lipase respectively have been tested. The amino acid sequences of these further proteins represent variants of the amino acid sequence represented by the amino acid in SEQ ID No. 1, wherein:
in the amino acid sequence shown under SEQ ID No. 1 the three amino acids at positions 40, 79 and 186 are different from the amino acids given at the respective amino acid positions in the sequence shown under SEQ ID No. 1. In a particular tested variant, with reference to the amino acid sequence shown under SEQ ID No. 1, the amino acid at position 40 is M and the amino acid at position 79 is S and the amino acid at position 186 is Y;
in the amino acid sequence shown under SEQ ID No. 1 the three amino acids at positions 40, 79 and 301 are different from the amino acids given at the respective amino acid positions in the sequence shown under SEQ ID No. 1. In a particular tested variant, with reference to the amino acid sequence shown under SEQ ID No. 1, the amino acid at position 40 is M and the amino acid at position 79 is S and the amino acid at position 301 is A;
in the amino acid sequence shown under SEQ ID No. 1 the three amino acids at positions 40, 186 and 301 are different from the amino acids given at the respective amino acid positions in the sequence shown under SEQ ID No. 1. In a particular tested variant, with reference to the amino acid sequence shown under SEQ ID No. 1, the amino acid at position 40 is M and the amino acid at position 186 is Y and the amino acid at position 301 is A;
in the amino acid sequence shown under SEQ ID No. 1 the three amino acids at positions 79, 186 and 301 are different from the amino acids given at the respective amino acid positions in the sequence shown under SEQ ID No. 1. In a particular tested variant, with reference to the amino acid sequence shown under SEQ ID No. 1, the amino acid at position 79 is S and the amino acid at position 186 is Y and the amino acid at position 301 is A;

The lipase variants or protein variants according to the invention show improved selectivity and/or improved specific activity regarding the stereoselective acylation or carboxylation of 2,6-Dimethyl-1-indanamine (DMAI) and are better adapted to produce enantiomerically enriched or nearly pure methyl-[(1R,2S)-2,6-dimethyl-2,3-dihydro-1H-inden-1-yl]carbamate compared to the wild-type lipase. Methyl-[(1R,2S)-2,6-dimethyl-2,3-dihydro-1H-inden-1-yl]carbamate is an important intermediate for the synthesis of the herbicidal active compound Indaziflam.

“Enantiomerically enriched” means herein that one of two enantiomers is present in a composition in higher amounts than the other enantiomer, preferably at least 60% of one enantiomer is present in the composition, more preferably at least 65% of one enantiomer is present in the composition, further more preferably at least 70% of one enantiomer is present in the composition, even more preferably at least 75% of one enantiomer is present in the composition, even further more preferably at least 80% of one enantiomer is present in the composition, particular preferably at least 85% of one enantiomer is present in the composition, most preferably at least 90% of one enantiomer is present in the composition or especially preferably at least 94% of one enantiomer is present in the composition.

“Enantiomerically nearly pure” means herein that one of two enantiomers is present in a composition in amounts of at least 95.0%, preferably one of two enantiomers is present in a composition in amounts of at least 95.5%, more preferably one of two enantiomers is present in a composition in amounts of at least 96.0%, further more preferably one of two enantiomers is present in a composition in amounts of at least 96.5%, even more preferably one of two enantiomers is present in a composition in amounts of at least 97.0%, even further more preferably one of two enantiomers is present in a composition in amounts of at least 98.0%, particular preferably one of two enantiomers is present in a composition in amounts of at least 98.5%, most preferably one of two enantiomers is present in a composition in amounts of at least 99.0%, or especially preferably one of two enantiomers is present in a composition in amounts of at least 99.5%.

One further embodiment of the invention concerns nucleic acid molecules encoding a protein according to the invention.

Nucleic acid molecules according to the invention can be any kind of nucleic acid, as long as the nucleic acid encodes a protein according to the invention. The nucleic acids can be ribonucleic nucleic acid molecules (e.g. RNA, mRNA) or deoxyribonucleic nucleic acid molecules (DNA, including genomic DNA which may or may not comprise introns and coding DNA).

Of particular interest for the invention are nucleic acid molecules encoding proteins having the activity of a lipase comprising the amino acid sequences shown under SEQ ID Nos. 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401.

The invention therefore also concerns nucleic acid molecules encoding a protein having the activity of a lipase selected from the group consisting of
a) nucleic acid molecules comprising the nucleic acid sequences shown under SEQ ID Nos. 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402;
b) nucleic acid molecules having at least 60%, preferably 70%, more preferably 80%, further more preferably 90%, even more preferably 95%, even further more preferably 96%, particular preferably 97%, most preferably 98% or especially preferably 99% identity with the nucleic acid sequences shown under a).

In the context of the present invention, the term “hybridizing with” means hybridization under conventional hybridization conditions, preferably under stringent conditions, as described, for example, in Sambrook et al. (Molecular Cloning, A Laboratory Manual, 3rd edition (2001) Cold Spring Harbor Laboratory Press, Cold Spring Harbor, NY. ISBN: 0879695773) or Ausubel et al. (Short Protocols in Molecular Biology, John Wiley & Sons; 5th edition (2002), ISBN: 0471250929). With particular preference, “hybridization” means a hybridization under the following conditions:
hybridization buffer:
2×SSC; 10×Denhardt solution (Fikoll 400+PEG+BSA; ratio 1:1:1); 0.1% SDS; 5 mM EDTA; 50 mM Na2HPO4; 250 μg/ml of herring sperm DNA; 50 μg/ml of tRNA;
or
25 M sodium phosphate buffer pH 7.2; 1 mM EDTA; 7% SDS
hybridization temperature: T=65 to 68° C.
wash buffer: 0.1×SSC; 0.1% SDS
wash temperature: T=65 to 68° C.

Nucleic acid molecules which hybridize with nucleic acid molecules coding for a protein having the activity of a lipase may originate from any organism; accordingly, they may originate from bacteria, fungi, animals, humans, plants or viruses.

Nucleic acid molecules which hybridize with nucleic acid molecules coding for a protein having the activity of a lipase preferably originate from microorganisms, more preferably from fungi or bacteria, most preferably from bacteria.

Nucleic acid molecules which hybridize with the molecules mentioned may be isolated, for example, from genomic or from cDNA libraries. Such nucleic acid molecules can be identified and isolated using the nucleic acid molecules described herein or they can be identified and isolated using parts of these molecules or the reverse complements of these molecules, for example by hybridization according to standard methods (see, for example, Sambrook et al., Molecular Cloning, A Laboratory Manual, 3rd edition (2001) Cold Spring Harbor Laboratory Press, Cold Spring Harbor, NY. ISBN: 0879695773; Ausubel et al., Short Protocols in Molecular Biology, John Wiley & Sons; 5th edition (2002), ISBN: 0471250929) or by amplification using PCR.

The fragments used as hybridization samples may also be synthetic fragments or oligonucleotides prepared using the customary synthesis techniques, whose sequence is essentially identical to the nucleic acid molecule described in the context of the present invention. Once genes which hybridize with the nucleic acid sequences described in the context of the present invention are identified and isolated, the sequence should be determined and the properties of the proteins coded for by this sequence should be analyzed to determine whether they are proteins having the activity of a lipase. Methods of how to determine whether a protein has the activity of a protein having the activity of a lipase are known to the person skilled in the art.

The molecules hybridizing with the nucleic acid molecules described in the context of the present invention comprise in particular fragments, derivatives and allelic variants of the nucleic acid molecules mentioned. In the context of the present invention, the term “derivative” means that the sequences of these molecules differ in one or more positions from the sequences of the nucleic acid molecules described above and are highly identical to these sequences. The differences to the nucleic acid molecules described above may, for example, be due to deletion, addition, substitution, insertion or recombination.

Preferred nucleic acid molecules according to the invention are those nucleic acid molecules shown under SEQ ID Nos. 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402.

The meaning of nucleotide abbreviations a, c, g, t, and those of abbreviations for degenerate nucleotides r, y, s, w, k, m, b, d, h, v, n is derivable herein below from Table 1 under the paragraph sub-titled “Description of the Sequences”. Which amino acids are encoded by codons comprising degenerate nucleotides is derivable herein below from Table 3 under the paragraph sub-titled “Description of the Sequences”.

Furthermore, the invention relates to recombinant nucleic acid molecules comprising a nucleic acid molecule according to the invention.

In conjunction with the present invention, the term “recombinant nucleic acid molecule” is to be understood to mean a nucleic acid molecule, which contains additional sequences in addition to nucleic acid molecules according to the invention, which do not naturally occur in the combination in which they occur in recombinant nucleic acids according to the invention. Here, the abovementioned additional sequences can be any sequences, preferably they are functional or regulatory sequences (promoters, termination signals, enhancers, ribosome binding sites (rbs), leader sequences enhancing transcription, translation or RNA stability, subcellular targeting sequences etc.), particularly preferably they are functional or regulatory sequences that are active in microorganisms, and especially particularly preferably they are regulatory sequences that are active in fungi, in particular yeasts or in bacteria.

Methods for the creation of recombinant nucleic acid molecules according to the invention are known to the person skilled in the art and include genetic methods such as bonding nucleic acid molecules by way of ligation, genetic recombination, or new synthesis of nucleic acid molecules. Those methods are described e.g. in Sambrok et al. (Molecular Cloning, A Laboratory Manual, 3rd edition (2001) Cold Spring Harbour Laboratory Press, Cold Spring Harbour, NY. ISBN: 0879695773) or Ausubel et al. (Short Protocols in Molecular Biology, John Wiley & Sons; 5th edition (2002), ISBN: 0471250929).

In a further embodiment, the recombinant nucleic acid molecules according to the invention comprise a nucleic acid molecule according to the invention which is linked with regulatory sequences, which initiate transcription in prokaryotic or eukaryotic cells.

Regulatory sequences, which initiate transcription” in a cell are also known as promoters.

Information concerning regulatory sequences and plasmids are well known to a person skilled in the art and are described e.g. by the Registry of Standard Biological Parts supported by The International Genetically Engineered Machine (iGEM) Foundation (One Kendall Square, Suite B6104, Cambridge, MA 02139, USA) in the world wide web (http://parts.igem.org/Catalog).

Regulatory sequences which initiate transcription in prokaryotic organisms, e.g. E. coli, and in eukaryotic organisms are sufficiently described in literature, in particular such for expression in yeast are described, e.g. Saccharomyces cerevisiae. An overview of various systems for expression for proteins in various host organisms can be found, for example, in Methods in Enzymology 153 (1987), 383-516 and in Bitter et al. (Methods in Enzymology 153 (1987), 516-544) or in Gomes et al. (2016, Advances in Animal and Veterinary Sciences, 4(4), 346) and Baghban et al. (2018, Current Pharmaceutical Biotechnology, 19(6)). Common yeast promoters are pAOX1, pHIS4, pGAL, pScADH2 (Baghban et al., 2018, see above). Common bacterial promoters are T5, T7, rhamnose-inducible, arabinose-inducible, PhoA, artificial trc (trp-lac) promoter as described by Marschall et al. (2017, Appl Microbiol Biotechnol 101, 501-512) and Tegel et al. (2011, FEBS Journal 278, 729-739).

A further embodiment of recombinant nucleic acid molecules of the present invention are vectors or plasmids, which comprise the nucleic acid molecules according to the invention.

“Vectors” are commonly understood in the field of molecular biology and herein to represent a nucleic acid sequence or a vehicle comprising a nucleic acid sequence used to transfer genetic material (DNA or RNA) into a target cell. Vectors can be plasmids, e.g. T-DNA or binary vectors for generating transgenic plants, expression vectors for expression of nucleic acid sequences in a host cell, shuttle vectors which are eligible to propagate in different hosts, or vectors can be virus particles or bacteriophages having been modified to deliver foreign genetic material into a host.

“Plasmids” are commonly understood in the field of molecular biology and herein to represent an autonomously self-replicating, often circular DNA molecule which is when present in a host cell separated from the chromosomal DNA.

Nucleic acid molecules according to the invention, recombinant nucleic acid molecules according to the invention, vectors or plasmids according to the invention can be used for production of proteins according to the invention, e.g. by expressing the nucleic acid molecules according to the invention in host cells.

Another embodiment of the invention concerns hosts or host cells comprising or expressing a nucleic acid molecule according to invention or comprising proteins according to the invention or comprising a recombinant nucleic acid molecule according to the invention or comprising a vector according to the invention or comprising a plasmid according to the invention.

Nucleic acid molecules according to the invention encoding a protein having the activity of a lipase can be expressed in host cells for e.g. their multiplication or for production of proteins according to the invention. For expression in host cells, nucleic acid molecules according to the invention can be comprised on vectors or plasmids or they can be stably integrated into the genome of a respective host cell. The nucleic acid molecules according to the invention can also be comprised by vectors which support their introduction into host cells.

A further embodiment of the present invention concerns a host or host cell according to the invention comprising a nucleic acid molecule according to the invention or comprising a recombinant nucleic acid molecule according to the invention or comprising a vector according to the invention or comprising a plasmid according to the invention and, in each case comprising a protein according to the invention.

Another embodiment of the present invention concerns a host or host cell according to the invention comprising a nucleic acid molecule according to the invention or comprising a recombinant nucleic acid molecule according to the invention or comprising a vector according to the invention or comprising a plasmid according to the invention and, in each case expressing a protein according to the invention.

Another embodiment of the present invention concerns a host or host cell according to the invention comprising a nucleic acid molecule according to the invention or comprising a recombinant nucleic acid molecule according to the invention or comprising a vector according to the invention or comprising a plasmid according to the invention and, in each case expressing a protein, wherein the protein has the activity of a lipase.

“Expressing a nucleic acid molecule” shall be understood herein to mean that in case the nucleic acid molecule is RNA or mRNA the nucleic acid molecule is translated into a protein, preferably translated into a protein having the activity of a lipase or in case of the nucleic acid molecule is DNA or cDNA it is transcribed (and in case of genomic DNA containing introns is processed) into mRNA, preferably into a mRNA encoding a protein having the activity of a lipase and subsequently translated into a protein, preferably translated into a protein having the activity of a lipase.

Transcription of a given nucleic acid molecule in a host can be demonstrated by methods known to a person skilled in the art, for example, by detection of specific transcripts (mRNA) of foreign nucleic acid molecules by Northern blot analysis or RT-PCR.

Whether hosts or host cells comprise a given protein or comprise a protein which is derived from expressing a nucleic acid molecule can be determined by methods known to a person skilled in the art, for example, by immunological methods, such as Western blot analysis, ELISA (Enzyme Linked Immuno Sorbent Assay) or RIA (Radio Immune Assay). The person skilled in the art is familiar with methods for preparing antibodies which react specifically with a certain protein, i.e. which bind specifically to a certain protein (see, for example, Lottspeich and Zorbas (eds.), 1998, Bioanalytik, Spektrum akad, Verlag, Heidelberg, Berlin, ISBN 3-8274-0041-4). Some companies (Thermo Fisher Scientific, 168 Third Avenue, Waltham, MA USA 0245; GenScript, 60 Centennial Ave., Piscataway, NJ 08854, USA) offer the preparation of such antibodies as an order service.

Furthermore, a person skilled in the art can test if a host or host cell comprises a protein according to the invention by detecting (additional) activity of proteins having the activity of a lipase in a respective host cell. Preferably activity of proteins having additional activity of a lipase in a respective host cell is detected by comparing the activities of lipases of a host cell according to the invention with the respective activity of host cell not comprising a protein according to the invention.

Testing if a protein has the activity of a lipase can be done by methods known in the art.

Host or host cells according to the invention can be produced by a person skilled in the art by known methods for genetically modifying or transforming organisms.

A further subject of the present invention therefore is a host or host cell according to the invention, particularly a prokaryotic or eukaryotic host or host cell, which is genetically modified (or transformed) with a nucleic acid molecule according to the invention or with a recombinant nucleic acid molecule according to the invention or with a vector according to the invention or a plasmid according to the invention. Preferably the genetically modified (transformed) host or host cell according to the invention expresses a protein having the activity of a lipase, more preferably, the genetically modified (transformed) host or host cell according to the invention expresses a protein according to the invention.

“Genetically modified with a nucleic acid molecule” or “transformed with a nucleic acid molecule” shall be understood herein to mean that a nucleic acid molecule is or was introduced into a host or host cell by technical and/or non-naturally occurring means, preferably by technical methods in the field of molecular biology, biotechnology or genetic modification.

Descendants, offspring or progeny of hosts or host cells according to the invention are also an embodiment of the invention, preferably these descendants, offspring or progeny comprise a nucleic acid molecule according to the invention or comprise a recombinant nucleic acid molecule according to the invention or comprise a vector according to the invention or comprise a plasmid according to the invention or comprise a protein according to the invention, more preferably these descendants, offspring or progeny comprise a nucleic acid molecule according to the invention or comprise a recombinant nucleic acid molecule according to the invention or comprise a vector according to the invention or comprise a plasmid according to the invention and, in each case express a protein, wherein the protein has the activity of a lipase, even more preferably these descendants, offspring or progeny comprise a nucleic acid molecule according to the invention or comprise a recombinant nucleic acid molecule according to the invention or comprise a vector according to the invention or comprise a plasmid according to the invention and, in each case express a protein, wherein the protein has the activity of a lipase according to the invention.

The host or host cell according to the invention can be a host or host cell from any prokaryotic or eucaryotic organism. The hosts or host cells can be bacteria or bacteria cells (e.g. E. coli, bacteria of the genus Bacillus, in particular Bacillus subtilis, Agrobacterium, particularly Agrobacterium tumefaciens or Agrobacterium rhizogenes, Pseudomonas, particularly Pseudomonas fluorescens, Streptomyces spp, Rhodococcus spp, in particular Rhodococcus rhodochrous, Vibrio natrigens, Corynebacterium, particularly Corynebacterium glutamicum) or fungi or fungal cells (e.g. Agaricus, in particular Agaricus bisporus, Aspergillus, Trichoderma or yeasts, particularly S. cerevisiae, Pichia ssp. like P. pastoris), as well as plants or plant cells or they can be animals or animal cells.

Preferred host cells according to the invention are cells of microorganisms. Within the framework of the present patent application, this is understood to include all bacteria and all protists (e.g. fungi, particularly yeasts and algae), as they are defined in Schlegel “General Microbiology” (Georg Thieme Publishing House (1985), 1-2), for example.

In respect to microorganisms, the hosts or host cells according to the invention are preferably bacteria/bacteria cells or yeast/yeast cells, most preferably they are bacteria/bacteria cells. Concerning bacteria/bacteria cells, the hosts or host cells according to the invention are preferably Bacillus species/Bacillus species cells or Escherichia coli/Escherichia coli cells cells most preferably Escherichia coli/Escherichia coli cells.

Alternatively, Pseudomonas, particularly Pseudomonas fluorescens, Streptomyces spp, Rhodococcus spp, in particular Rhodococcus rhodochrous, Vibrio spp, particularly Vibrio natrigens, Corynebacterium, particularly Corynebacterium glutamicum or others can be hosts or host cells according to the invention.

A preferred embodiment of the invention concerns hosts or host cells according to the invention comprising a nucleic acid molecule according to the invention, wherein the nucleic acid molecule according to the invention is characterized in that the codons of said nucleic acid molecule are changed such that they are adapted to the frequency of use of the codons of the host or a host cell, respectively.

Host cells according to the invention can be used for production of proteins according to the invention. Proteins according to the invention can be used in methods for production of enantiomerically enriched or nearly enantiomerically pure acylated or carboxylated products from a acyl or carboxyl acceptor in the presence of an acyl or carboxyl donor.

Another embodiment of the invention therefore concerns a method for the production of an acylated or carboxylated product—comprising the steps of
a) providing an acyl or carboxyl acceptor molecule;
b) providing an acyl or carboxyl donor molecule;
c) contacting the acyl or carboxyl acceptor molecule provided in step a) and the acyl or carboxyl donor molecule provided in step b) with a protein according to the invention;
d) optionally, obtaining the acylated or carboxylated product.

A further embodiment of the invention is the use of a protein according to the invention production of an amine, preferably for production of an (1R,2S)-amine.

A further embodiment of the invention is the use of a protein according to the invention for the stereoselective acylation or carboxylation of racemic 2,6-dimethyl-1-indanylamine.

A further embodiment of the invention is the use of a protein according to the invention for the stereoselective acylation or carboxylation of racemic 2,6-dimethyl-1-indanylamine, wherein the substrate is racemic 2,6-dimethyl-1-indanylamine.

### Description of the Sequences

Throughout the application, nucleotide and amino acid abbreviations are used according to the following IUPAC codes:

TABLE 1

| IUPAC nucleotide | |
|---|---|
| code | Base |
| A | Adenine |
| C | Cytosine |
| G | Guanine |
| T (or U) | Thymine (or Uracil) |
| R | A or G |
| Y | C or T |
| S | G or C |
| W | A or T |
| K | G or T |
| M | A or C |
| B | C or G or T |
| D | A or G or T |
| H | A or C or T |
| V | A or C or G |
| N | any base |
| — | gap |

For discrimination between amino acids and nucleotides, capitalized nucleotide code abbreviations given in above Table are written herein in lower case.

TABLE 2

| IUPAC | Three | |
|---|---|---|
| amino acid | letter | |
| code | code | Amino acid |
| A | Ala | Alanine |
| C | Cys | Cysteine |
| D | Asp | Aspartic Acid |
| E | Glu | Glutamic Acid |
| F | Phe | Phenylalanine |
| G | Gly | Glycine |
| H | His | Histidine |
| I | Ile | Isoleucine |
| K | Lys | Lysine |
| L | Leu | Leucine |
| M | Met | Methionine |
| N | Asn | Asparagine |
| P | Pro | Proline |
| Q | Gln | Glutamine |
| R | Arg | Arginine |
| S | Ser | Serine |
| T | Thr | Threonine |
| V | Val | Valine |
| W | Trp | Tryptophan |
| Y | Tyr | Tyrosine |

Codon usage follows herein the so called “general genetic code” according to the following Table, wherein “t” is to be substituted by “u” in ribonucleic acid (RNA) sequences.

TABLE 3

| | | | | Codons |
|---|---|---|---|---|
| | | | | due to |
| | Three | Single | | degenerate |
| | letter | letter | DNA | genetic |
| Amino Acid | code | code | codons | code |
| Alanine | Ala | A | gca | gcn |
| Alanine | Ala | A | gcc | gcn |
| Alanine | Ala | A | gcg | gcn |
| Alanine | Ala | A | gct | gcn |
| Arginine | Arg | R | aga | mgn |
| Arginine | Arg | R | agg | mgn |
| Arginine | Arg | R | cga | mgn |
| Arginine | Arg | R | cgc | mgn |
| Arginine | Arg | R | cgg | mgn |
| Arginine | Arg | R | cgt | mgn |
| Asparagine | Asn | N | aac | aay |
| Asparagine | Asn | N | aat | aay |
| Aspartic acid | Asp | D | gac | gay |
| Aspartic acid | Asp | D | gat | gay |
| Cysteine | Cys | C | tgc | tgy |
| Cysteine | Cys | C | tgt | tgy |
| Glutamic acid | Glu | E | gaa | gar |
| Glutamic acid | Glu | E | gag | gar |
| Glutamine | Gln | Q | caa | car |
| Glutamine | Gln | Q | cag | car |
| Glycine | Gly | G | gga | ggn |
| Glycine | Gly | G | ggc | ggn |
| Glycine | Gly | G | ggg | ggn |
| Glycine | Gly | G | ggt | ggn |
| Histidine | His | H | cac | cay |
| Histidine | His | H | cat | cay |
| Isoleucine | Ile | I | ata | ath |
| Isoleucine | Ile | I | atc | ath |
| Isoleucine | Ile | I | att | ath |
| Leucine | Leu | L | cta | ytn |
| Leucine | Leu | L | ctc | ytn |
| Leucine | Leu | L | ctg | ytn |
| Leucine | Leu | L | ctt | ytn |
| Leucine | Leu | L | tta | ytn |
| Leucine | Leu | L | ttg | ytn |
| Lysine | Lys | K | aaa | aar |
| Lysine | Lys | K | aag | aar |
| Methionine | Met | M | atg | atg |
| Phenylalanine | Phe | F | ttc | tty |
| Phenylalanine | Phe | F | ttt | tty |
| Proline | Pro | P | cca | ccn |
| Proline | Pro | P | ccc | con |
| Proline | Pro | P | ccg | ccn |
| Proline | Pro | P | cct | ccn |
| Serine | Ser | S | agc | wsn |
| Serine | Ser | S | agt | wsn |
| Serine | Ser | S | tca | wsn |
| Serine | Ser | S | tcc | wsn |
| Serine | Ser | S | tcg | wsn |
| Serine | Ser | S | tct | wsn |
| Threonine | Thr | T | aca | acn |
| Threonine | Thr | T | acc | acn |
| Threonine | Thr | T | acg | acn |
| Threonine | Thr | T | act | acn |
| Tryptophan | Thr | W | tgg | tgg |
| Tyrosine | Tyr | Y | tac | tay |
| Tyrosine | Tyr | Y | tat | tay |
| Valine | Val | V | gta | gtn |
| Valine | Val | V | gtc | gtn |
| Valine | Val | V | gtg | gtn |
| Valine | Val | V | gtt | gtn |
| Stop codons | Stop | Stop | taa | trr |
| Stop codons | Stop | Stop | tag | trr |
| Stop codons | Stop | Stop | tga | trr |

TABLE 4
The Sequence listing associated with this application is filed in
electronic format and hereby incorporated by reference into the
specification in its entirety. “PRT” stands for “protein” and
“NUC” for “nucleic acid”.

| SEQ | | | |
|---|---|---|---|
| ID | | | |
| No | Subject | Mutations/Variations | Type |
| 1 | wild-type (WT) | — | PRT |
| 2 | wild-type (WT) | — | NUC |
| 3 | Lipase variant | L186F | PRT |
| 4 | Lipase variant | L186F | NUC |
| 5 | Lipase variant | L280E | PRT |
| 6 | Lipase variant | L280E | NUC |
| 7 | Lipase variant | L280S | PRT |
| 8 | Lipase variant | L280S | NUC |
| 9 | Lipase variant | L280K | PRT |
| 10 | Lipase variant | L280K | NUC |
| 11 | Lipase variant | P312N | PRT |
| 12 | Lipase variant | P312N | NUC |
| 13 | Lipase variant | P312F | PRT |
| 14 | Lipase variant | P312F | NUC |
| 15 | Lipase variant | L186W | PRT |
| 16 | Lipase variant | L186W | NUC |
| 17 | Lipase variant | M3L | PRT |
| 18 | Lipase variant | M3L | NUC |
| 19 | Lipase variant | N29Y | PRT |
| 20 | Lipase variant | N29Y | NUC |
| 21 | Lipase variant | L17P | PRT |
| 22 | Lipase variant | L17P | NUC |
| 23 | Lipase variant | S4P | PRT |
| 24 | Lipase variant | S4P | NUC |
| 25 | Lipase variant | L280D | PRT |
| 26 | Lipase variant | L280D | NUC |
| 27 | Lipase variant | V18A | PRT |
| 28 | Lipase variant | V18A | NUC |
| 29 | Lipase variant | M3Q | PRT |
| 30 | Lipase variant | M3Q | NUC |
| 31 | Lipase variant | P312D | PRT |
| 32 | Lipase variant | P312D | NUC |
| 33 | Lipase variant | N29W | PRT |
| 34 | Lipase variant | N29W | NUC |
| 35 | Lipase variant | L186Y | PRT |
| 36 | Lipase variant | L186Y | NUC |
| 37 | Lipase variant | V18T | PRT |
| 38 | Lipase variant | V18T | NUC |
| 39 | Lipase variant | L186E | PRT |
| 40 | Lipase variant | L186E | NUC |
| 41 | Lipase variant | A202Q | PRT |
| 42 | Lipase variant | A202Q | NUC |
| 43 | Lipase variant | D301A | PRT |
| 44 | Lipase variant | D301A | NUC |
| 45 | Lipase variant | P309C | PRT |
| 46 | Lipase variant | P309C | NUC |
| 47 | Lipase variant | L186D | PRT |
| 48 | Lipase variant | L186D | NUC |
| 49 | Lipase variant | Q31W | PRT |
| 50 | Lipase variant | Q31W | NUC |
| 51 | Lipase variant | A202N | PRT |
| 52 | Lipase variant | A202N | NUC |
| 53 | Lipase variant | Q111E | PRT |
| 54 | Lipase variant | Q111E | NUC |
| 55 | Lipase variant | L186Q | PRT |
| 56 | Lipase variant | L186Q | NUC |
| 57 | Lipase variant | L186T | PRT |
| 58 | Lipase variant | L186T | NUC |
| 59 | Lipase variant | M3C | PRT |
| 60 | Lipase variant | M3C | NUC |
| 61 | Lipase variant | W85H | PRT |
| 62 | Lipase variant | W85H | NUC |
| 63 | Lipase variant | N29H | PRT |
| 64 | Lipase variant | N29H | NUC |
| 65 | Lipase variant | K8E | PRT |
| 66 | Lipase variant | K8E | NUC |
| 67 | Lipase variant | L186H | PRT |
| 68 | Lipase variant | L186H | NUC |
| 69 | Lipase variant | E79I | PRT |
| 70 | Lipase variant | E79I | NUC |
| 71 | Lipase variant | E79W | PRT |
| 72 | Lipase variant | E79W | NUC |
| 73 | Lipase variant | L17T | PRT |
| 74 | Lipase variant | L17T | NUC |
| 75 | Lipase variant | V18C | PRT |
| 76 | Lipase variant | V18C | NUC |
| 77 | Lipase variant | L186P | PRT |
| 78 | Lipase variant | L186P | NUC |
| 79 | Lipase variant | P312Q | PRT |
| 80 | Lipase variant | P312Q | NUC |
| 81 | Lipase variant | K40M | PRT |
| 82 | Lipase variant | K40M | NUC |
| 83 | Lipase variant | P312K | PRT |
| 84 | Lipase variant | P312K | NUC |
| 85 | Lipase variant | L186C | PRT |
| 86 | Lipase variant | L186C | NUC |
| 87 | Lipase variant | L280A | PRT |
| 88 | Lipase variant | L280A | NUC |
| 89 | Lipase variant | S4L | PRT |
| 90 | Lipase variant | S4L | NUC |
| 91 | Lipase variant | V18S | PRT |
| 92 | Lipase variant | V18S | NUC |
| 93 | Lipase variant | L186K | PRT |
| 94 | Lipase variant | L186K | NUC |
| 95 | Lipase variant | L186S | PRT |
| 96 | Lipase variant | L186S | NUC |
| 97 | Lipase variant | E79T | PRT |
| 98 | Lipase variant | E79T | NUC |
| 99 | Lipase variant | L186N | PRT |
| 100 | Lipase variant | L186N | NUC |
| 101 | Lipase variant | L186I | PRT |
| 102 | Lipase variant | L186I | NUC |
| 103 | Lipase variant | L186V | PRT |
| 104 | Lipase variant | L186V | NUC |
| 105 | Lipase variant | E79S, L186Y | PRT |
| 106 | Lipase variant | E79S, L186Y | NUC |
| 107 | Lipase variant | C11A, L186Y, P312N | PRT |
| 108 | Lipase variant | C11A, L186Y, P312N | NUC |
| 109 | Lipase variant | L186Y, L280A, P312N | PRT |
| 110 | Lipase variant | L186Y, L280A, P312N | NUC |
| 111 | Lipase variant | L17P, L186Y, P312N | PRT |
| 112 | Lipase variant | L17P, L186Y, P312N | NUC |
| 113 | Lipase variant | L186W, L280A, P312N | PRT |
| 114 | Lipase variant | L186W, L280A, P312N | NUC |
| 115 | Lipase variant | N29H, L186W, L280A | PRT |
| 116 | Lipase variant | N29H, L186W, L280A | NUC |
| 117 | Lipase variant | L17P, L186Y, P309C | PRT |
| 118 | Lipase variant | L17P, L186Y, P309C | NUC |
| 119 | Lipase variant | S4P, L186W, A202N | PRT |
| 120 | Lipase variant | S4P, L186W, A202N | NUC |
| 121 | Lipase variant | L186W, A202N, P312N | PRT |
| 122 | Lipase variant | L186W, A202N, P312N | NUC |
| 123 | Lipase variant | E79S, L186W, P309C, P312N | PRT |
| 124 | Lipase variant | E79S, L186W, P309C, P312N | NUC |
| 125 | Lipase variant | L186Y, F192A, A202N, P312N | PRT |
| 126 | Lipase variant | L186Y, F192A, A202N, P312N | NUC |
| 127 | Lipase variant | K8E, C11A, L186W, P312N | PRT |
| 128 | Lipase variant | K8E, C11A, L186W, P312N | NUC |
| 129 | Lipase variant | E79S, L186Y, P309C, P312N | PRT |
| 130 | Lipase variant | E79S, L186Y, P309C, P312N | NUC |
| 131 | Lipase variant | E79S, N84T, L186W, P309C | PRT |
| 132 | Lipase variant | E79S, N84T, L186W, P309C | NUC |
| 133 | Lipase variant | E79S, L186W, F192A, P312N | PRT |
| 134 | Lipase variant | E79S, L186W, F192A, P312N | NUC |
| 135 | Lipase variant | N29H, N84T, L186Y, A202N | PRT |
| 136 | Lipase variant | N29H, N84T, L186Y, A202N | NUC |
| 137 | Lipase variant | V18C, K40M, N84T, L186W, | PRT |
| | | P312N | |
| 138 | Lipase variant | V18C, K40M, N84T, L186W, | NUC |
| | | P312N | |
| 139 | Lipase variant | S4P, N29H, L186Y, A202N, | PRT |
| | | P312N | |
| 140 | Lipase variant | S4P, N29H, L186Y, A202N, | NUC |
| | | P312N | |
| 141 | Lipase variant | V18A, L186Y, F192A, D301A, | PRT |
| | | P312N | |
| 142 | Lipase variant | V18A, L186Y, F192A, D301A, | NUC |
| | | P312N | |
| 143 | Lipase variant | N29H, Q111E, L186W, P309C, | PRT |
| | | P312N | |
| 144 | Lipase variant | N29H, Q111E, L186W, P309C, | NUC |
| | | P312N | |
| 145 | Lipase variant | M3Q, S4P, K8E, N84T, L186W, | PRT |
| | | P312N | |
| 146 | Lipase variant | M3Q, S4P, K8E, N84T, L186W, | NUC |
| | | P312N | |
| 147 | Lipase variant | E79S, L186Y, D301A | PRT |
| 148 | Lipase variant | E79S, L186Y, D301A | NUC |
| 149 | Lipase variant | L186Y, A202N, D301A | PRT |
| 150 | Lipase variant | L186Y, A202N, D301A | NUC |
| 151 | Lipase variant | L17P, L186W, A202N | PRT |
| 152 | Lipase variant | L17P, L186W, A202N | NUC |
| 153 | Lipase variant | M3Q, E79S, L186Y, P312N | PRT |
| 154 | Lipase variant | M3Q, E79S, L186Y, P312N | NUC |
| 155 | Lipase variant | K40M, E79S, L186Y, P312N | PRT |
| 156 | Lipase variant | K40M, E79S, L186Y, P312N | NUC |
| 157 | Lipase variant | K8E, L186Y, L280A, D301A | PRT |
| 158 | Lipase variant | K8E, L186Y, L280A, D301A | NUC |
| 159 | Lipase variant | L17P, E79S, L186Y, P309C | PRT |
| 160 | Lipase variant | L17P, E79S, L186Y, P309C | NUC |
| 161 | Lipase variant | L186Y, F192A, L280A, D301A | PRT |
| 162 | Lipase variant | L186Y, F192A, L280A, D301A | NUC |
| 163 | Lipase variant | L186Y, A202N, L280A, P312N | PRT |
| 164 | Lipase variant | L186Y, A202N, L280A, P312N | NUC |
| 165 | Lipase variant | K40M, L186W, L280A, P309C | PRT |
| 166 | Lipase variant | K40M, L186W, L280A, P309C | NUC |
| 167 | Lipase variant | C11A, N29W, E79S, L186Y | PRT |
| 168 | Lipase variant | C11A, N29W, E79S, L186Y | NUC |
| 169 | Lipase variant | N29W, L186Y, A202N, D301A | PRT |
| 170 | Lipase variant | N29W, L186Y, A202N, D301A | NUC |
| 171 | Lipase variant | L17P, N29H, E79S, L186Y | PRT |
| 172 | Lipase variant | L17P, N29H, E79S, L186Y | NUC |
| 173 | Lipase variant | K8E, C11A, L186Y, D301A | PRT |
| 174 | Lipase variant | K8E, C11A, L186Y, D301A | NUC |
| 175 | Lipase variant | S4P, E79S, L186W, A202N, | PRT |
| | | P309C | |
| 176 | Lipase variant | S4P, E79S, L186W, A202N, | NUC |
| | | P309C | |
| 177 | Lipase variant | Q31W, E79S, L186W, A202N, | PRT |
| | | P309C | |
| 178 | Lipase variant | Q31W, E79S, L186W, A202N, | NUC |
| | | P309C | |
| 179 | Lipase variant | E79S, L186W, L280A, P309C, | PRT |
| | | P312N | |
| 180 | Lipase variant | E79S, L186W, L280A, P309C, | NUC |
| | | P312N | |
| 181 | Lipase variant | Q31W, E79S, L186W, F192A, | PRT |
| | | D301A | |
| 182 | Lipase variant | Q31W, E79S, L186W, F192A, | NUC |
| | | D301A | |
| 183 | Lipase variant | Q31W, E79S, N84T, L186W, | PRT |
| | | A202N | |
| 184 | Lipase variant | Q31W, E79S, N84T, L186W, | NUC |
| | | A202N | |
| 185 | Lipase variant | E79S, N84T, L186Y, F192A, | PRT |
| | | L280A | |
| 186 | Lipase variant | E79S, N84T, L186Y, F192A, | NUC |
| | | L280A | |
| 187 | Lipase variant | E79S, N84T, L186W, F192A, | PRT |
| | | L280A | |
| 188 | Lipase variant | E79S, N84T, L186W, F192A, | NUC |
| | | L280A | |
| 189 | Lipase variant | S4P, L17P, N29W, L186Y, | PRT |
| | | D301A | |
| 190 | Lipase variant | S4P, L17P, N29W, L186Y, | NUC |
| | | D301A | |
| 191 | Lipase variant | N29H, E79S, L186W, F192A, | PRT |
| | | A202N | |
| 192 | Lipase variant | N29H, E79S, L186W, F192A, | NUC |
| | | A202N | |
| 193 | Lipase variant | E79S, W85H, L186Y, F192A, | PRT |
| | | D301A | |
| 194 | Lipase variant | E79S, W85H, L186Y, F192A, | NUC |
| | | D301A | |
| 195 | Lipase variant | L17P, N29H, E79S, L186Y, | PRT |
| | | P309C | |
| 196 | Lipase variant | L17P, N29H, E79S, L186Y, | NUC |
| | | P309C | |
| 197 | Lipase variant | N29H, Q31W, L186Y, L280A, | PRT |
| | | D301A | |
| 198 | Lipase variant | N29H, Q31W, L186Y, L280A, | NUC |
| | | D301A | |
| 199 | Lipase variant | S4P, C11A, W85H, L186Y, | PRT |
| | | A202N | |
| 200 | Lipase variant | S4P, C11A, W85H, L186Y, | NUC |
| | | A202N | |
| 201 | Lipase variant | S4P, E79S, W85H, L186Y, | PRT |
| | | L280A | |
| 202 | Lipase variant | S4P, E79S, W85H, L186Y, | NUC |
| | | L280A | |
| 203 | Lipase variant | K8E, K40M, E79S, N84T, | PRT |
| | | L186Y, P312N | |
| 204 | Lipase variant | K8E, K40M, E79S, N84T, | NUC |
| | | L186Y, P312N | |
| 205 | Lipase variant | Q31W, N84T, Q111E, L186Y, | PRT |
| | | L280A, P312N | |
| 206 | Lipase variant | Q31W, N84T, Q111E, L186Y, | NUC |
| | | L280A, P312N | |
| 207 | Lipase variant | Q31W, L186Y, F192A, L280A, | PRT |
| | | D301A, P312N | |
| 208 | Lipase variant | Q31W, L186Y, F192A, L280A, | NUC |
| | | D301A, P312N | |
| 209 | Lipase variant | S4P, K8E, N29W, E79S, | PRT |
| | | Q111E, L186W | |
| 210 | Lipase variant | S4P, K8E, N29W, E79S, | NUC |
| | | Q111E, L186W | |
| 211 | Lipase variant | L17P, V18A, L186W, L280A, | PRT |
| | | P309C, P312N | |
| 212 | Lipase variant | L17P, V18A, L186W, L280A, | NUC |
| | | P309C, P312N | |
| 213 | Lipase variant | E79S, N84T, Q111E, L186Y, | PRT |
| | | P309C, P312N | |
| 214 | Lipase variant | E79S, N84T, Q111E, L186Y, | NUC |
| | | P309C, P312N | |
| 215 | Lipase variant | N29H, L186Y, F192A, L280A, | PRT |
| | | D301A, P312N | |
| 216 | Lipase variant | N29H, L186Y, F192A, L280A, | NUC |
| | | D301A, P312N | |
| 217 | Lipase variant | K8E, C11A, N29H, Q31W, | PRT |
| | | L186Y, D301A | |
| 218 | Lipase variant | K8E, C11A, N29H, Q31W, | NUC |
| | | L186Y, D301A | |
| 219 | Lipase variant | S4P, N29H, Q31W, E79S, | PRT |
| | | L280A, D301A | |
| 220 | Lipase variant | S4P, N29H, Q31W, E79S, | NUC |
| | | L280A, D301A | |
| 221 | Lipase variant | K8E, N29H, Q31W, E79S, | PRT |
| | | L186W, D301A | |
| 222 | Lipase variant | K8E, N29H, Q31W, E79S, | NUC |
| | | L186W, D301A | |
| 223 | Lipase variant | K8E, L17P, V18A, Q111E, | PRT |
| | | L186W, P309C | |
| 224 | Lipase variant | K8E, L17P, V18A, Q111E, | NUC |
| | | L186W, P309C | |
| 225 | Lipase variant | C11A, N29W, E79S, N84T, | PRT |
| | | W85H, L186Y, P309C | |
| 226 | Lipase variant | C11A, N29W, E79S, N84T, | NUC |
| | | W85H, L186Y, P309C | |
| 227 | Lipase variant | N29H, N84T, L186Y, A202N, | PRT |
| | | L280A, P309C, P312N | |
| 228 | Lipase variant | N29H, N84T, L186Y, A202N, | NUC |
| | | L280A, P309C, P312N | |
| 229 | Lipase variant | N29H, Q31W, E79S, N84T, | PRT |
| | | W85H, L186Y, F192A, L280A, | |
| | | P309C | |
| 230 | Lipase variant | N29H, Q31W, E79S, N84T, | NUC |
| | | W85H, L186Y, F192A, L280A, | |
| | | P309C | |
| 231 | Lipase variant | L17P, E79S, L186W, A202N | PRT |
| 232 | Lipase variant | L17P, E79S, L186W, A202N | NUC |
| 233 | Lipase variant | K40M, E79S, L186Y, D301A | PRT |
| 234 | Lipase variant | K40M, E79S, L186Y, D301A | NUC |
| 235 | Lipase variant | E79S, L186W, A202N, L280A | PRT |
| 236 | Lipase variant | E79S, L186W, A202N, L280A | NUC |
| 237 | Lipase variant | L17P, Q111E, L186Y, D301A | PRT |
| 238 | Lipase variant | L17P, Q111E, L186Y, D301A | NUC |
| 239 | Lipase variant | L17P, E79S, L186Y, A202N | PRT |
| 240 | Lipase variant | L17P, E79S, L186Y, A202N | NUC |
| 241 | Lipase variant | M3Q, E79S, L186Y, D301A | PRT |
| 242 | Lipase variant | M3Q, E79S, L186Y, D301A | NUC |
| 243 | Lipase variant | E79S, L186W, L280A, D301A | PRT |
| 244 | Lipase variant | E79S, L186W, L280A, D301A | NUC |
| 245 | Lipase variant | L17P, E79S, Q111E, L186W, | PRT |
| | | P309C | |
| 246 | Lipase variant | L17P, E79S, Q111E, L186W, | NUC |
| | | P309C | |
| 247 | Lipase variant | L17P, V18C, E79S, L186W, | PRT |
| | | L280A | |
| 248 | Lipase variant | L17P, V18C, E79S, L186W, | NUC |
| | | L280A | |
| 249 | Lipase variant | N29H, K40M, E79S, L186W, | PRT |
| | | D301A | |
| 250 | Lipase variant | N29H, K40M, E79S, L186W, | NUC |
| | | D301A | |
| 251 | Lipase variant | N29H, E79S, L186W, A202N, | PRT |
| | | L280A | |
| 252 | Lipase variant | N29H, E79S, L186W, A202N, | NUC |
| | | L280A | |
| 253 | Lipase variant | N29H, E79S, Q111E, L186W, | PRT |
| | | L280A | |
| 254 | Lipase variant | N29H, E79S, Q111E, L186W, | NUC |
| | | L280A | |
| 255 | Lipase variant | N29H, E79S, L186W, A202N, | PRT |
| | | D301A | |
| 256 | Lipase variant | N29H, E79S, L186W, A202N, | NUC |
| | | D301A | |
| 257 | Lipase variant | C11A, L186Y, L280A, D301A, | PRT |
| | | P309C | |
| 258 | Lipase variant | C11A, L186Y, L280A, D301A, | NUC |
| | | P309C | |
| 259 | Lipase variant | K8E, E79S, L186Y, L280A, | PRT |
| | | D301A | |
| 260 | Lipase variant | K8E, E79S, L186Y, L280A, | NUC |
| | | D301A | |
| 261 | Lipase variant | L17P, N29W, Q111E, L186W, | PRT |
| | | A202N | |
| 262 | Lipase variant | L17P, N29W, Q111E, L186W, | NUC |
| | | A202N | |
| 263 | Lipase variant | L17P, L186Y, L280A, D301A, | PRT |
| | | P309C | |
| 264 | Lipase variant | L17P, L186Y, L280A, D301A, | NUC |
| | | P309C | |
| 265 | Lipase variant | Q111E, L186W, A202N, | PRT |
| | | L280A, P312N | |
| 266 | Lipase variant | Q111E, L186W, A202N, | NUC |
| | | L280A, P312N | |
| 267 | Lipase variant | N29H, E79S, Q111E, L186Y, | PRT |
| | | D301A | |
| 268 | Lipase variant | N29H, E79S, Q111E, L186Y, | NUC |
| | | D301A | |
| 269 | Lipase variant | L17P, N29W, E79S, L186Y, | PRT |
| | | A202N, P312N | |
| 270 | Lipase variant | L17P, N29W, E79S, L186Y, | NUC |
| | | A202N, P312N | |
| 271 | Lipase variant | L17P, E79S, N84T, L186Y, | PRT |
| | | L280A, P309C | |
| 272 | Lipase variant | L17P, E79S, N84T, L186Y, | NUC |
| | | L280A, P309C | |
| 273 | Lipase variant | L17P, N29H, E79S, W85H, | PRT |
| | | L186W, L280A | |
| 274 | Lipase variant | L17P, N29H, E79S, W85H, | NUC |
| | | L186W, L280A | |
| 275 | Lipase variant | L17P, E79S, L186Y, F192A, | PRT |
| | | A202N, P309C | |
| 276 | Lipase variant | L17P, E79S, L186Y, F192A, , | NUC |
| | | A202N P309C | |
| 277 | Lipase variant | V18A, K40M, E79S, L186W, | PRT |
| | | F192A, D301A | |
| 278 | Lipase variant | V18A, K40M, E79S, L186W, | NUC |
| | | F192A, D301A | |
| 279 | Lipase variant | L17P, N29H, Q31W, E79S, | PRT |
| | | L186W, D301A | |
| 280 | Lipase variant | L17P, N29H, Q31W, E79S, | NUC |
| | | L186W, D301A | |
| 281 | Lipase variant | K8E, K40M, Q111E, L186W, | PRT |
| | | L280A, P309C | |
| 282 | Lipase variant | K8E, K40M, Q111E, L186W, | NUC |
| | | L280A, P309C | |
| 283 | Lipase variant | M3Q, K8E, L17P, L186Y, | PRT |
| | | D301A, P312N | |
| 284 | Lipase variant | M3Q, K8E, L17P, L186Y, | NUC |
| | | D301A, P312N | |
| 285 | Lipase variant | L17P, N29W, L186W, A202N, | PRT |
| | | L280A, P309C | |
| 286 | Lipase variant | L17P, N29W, L186W, A202N, | NUC |
| | | L280A, P309C | |
| 287 | Lipase variant | K8E, C11A, L186W, A202N, | PRT |
| | | L280A, P309C | |
| 288 | Lipase variant | K8E, C11A, L186W, A202N, | NUC |
| | | L280A, P309C | |
| 289 | Lipase variant | S4P, N29W, E79S, L186W, | PRT |
| | | A202N, L280A, P309C | |
| 290 | Lipase variant | S4P, N29W, E79S, L186W, | NUC |
| | | A202N, L280A, P309C | |
| 291 | Lipase variant | N29W, E79S, Q111E, L186W, | PRT |
| | | L280A, P309C, P312N | |
| 292 | Lipase variant | N29W, E79S, Q111E, L186W, | NUC |
| | | L280A, P309C, P312N | |
| 293 | Lipase variant | K8E, C11A, K40M, E79S, | PRT |
| | | W85H, L186W, P309C | |
| 294 | Lipase variant | K8E, C11A, K40M, E79S, | NUC |
| | | W85H, L186W, P309C | |
| 295 | Lipase variant | M3Q, S4P, N29H, K40M, | PRT |
| | | L186Y, A202N, P309C | |
| 296 | Lipase variant | M3Q, S4P, N29H, K40M, | NUC |
| | | L186Y, A202N, P309C | |
| 297 | Lipase variant | V18A, K40M, N84T, Q111E, | PRT |
| | | L186Y, L280A, P309C, P312N | |
| 298 | Lipase variant | V18A, K40M, N84T, Q111E, | NUC |
| | | L186Y, L280A, P309C, P312N | |
| 299 | Lipase variant | S4P, C11A, V18A, K40M, | PRT |
| | | N84T, L186Y, D301A, P312N | |
| 300 | Lipase variant | S4P, C11A, V18A, K40M, | NUC |
| | | N84T, L186Y, D301A, P312N | |
| 301 | Lipase variant | S4P, K8E, C11A, L186Y, | PRT |
| | | F192A, A202N, L280A, P309C | |
| 302 | Lipase variant | S4P, K8E, C11A, L186Y, | NUC |
| | | F192A, A202N, L280A, P309C | |
| 303 | Lipase variant | E79S, Q111E, L186W, L280A, | PRT |
| | | D301A | |
| 304 | Lipase variant | E79S, Q111E, L186W, L280A, | NUC |
| | | D301A | |
| 305 | Lipase variant | M3Q, L17P, K40M, E79S, | PRT |
| | | L186Y | |
| 306 | Lipase variant | M3Q, L17P, K40M, E79S, | NUC |
| | | L186Y | |
| 307 | Lipase variant | L17P, K40M, Q111E, L186W, | PRT |
| | | L280A | |
| 308 | Lipase variant | L17P, K40M, Q111E, L186W, | NUC |
| | | L280A | |
| 309 | Lipase variant | C11A, E79S, L186Y, A202N, | PRT |
| | | D301A | |
| 310 | Lipase variant | C11A, E79S, L186Y, A202N, | NUC |
| | | D301A | |
| 311 | Lipase variant | L17P, K40M, E79S, Q111E, | PRT |
| | | L186Y | |
| 312 | Lipase variant | L17P, K40M, E79S, Q111E, | NUC |
| | | L186Y | |
| 313 | Lipase variant | C11A, L17P, K40M, E79S, | PRT |
| | | L186Y | |
| 314 | Lipase variant | C11A, L17P, K40M, E79S, | NUC |
| | | L186Y | |
| 315 | Lipase variant | K40M, Q111E, L186Y, L280A, | PRT |
| | | D301A | |
| 316 | Lipase variant | K40M, Q111E, L186Y, L280A, | NUC |
| | | D301A | |
| 317 | Lipase variant | E79S, N84T, L186W, A202N, | PRT |
| | | L280A, D301A | |
| 318 | Lipase variant | E79S, N84T, L186W, A202N, | NUC |
| | | L280A, D301A | |
| 319 | Lipase variant | M3Q, L17P, N29H, E79S, | PRT |
| | | L186Y, D301A | |
| 320 | Lipase variant | M3Q, L17P, N29H, E79S, | NUC |
| | | L186Y, D301A | |
| 321 | Lipase variant | L17P, Q111E, L186Y, A202N, | PRT |
| | | L280A, P312N | |
| 322 | Lipase variant | L17P, Q111E, L186Y, A202N, | NUC |
| | | L280A, P312N | |
| 323 | Lipase variant | M3Q, C11A, L186Y, A202N, | PRT |
| | | L280A, P312N | |
| 324 | Lipase variant | M3Q, C11A, L186Y, A202N, | NUC |
| | | L280A, P312N | |
| 325 | Lipase variant | L17P, E79S, Q111E, L186Y, | PRT |
| | | F192A, D301A | |
| 326 | Lipase variant | L17P, E79S, Q111E, L186Y, | NUC |
| | | F192A, D301A | |
| 327 | Lipase variant | C11A, E79S, W85H, Q111E, | PRT |
| | | L186Y, L280A, P312N | |
| 328 | Lipase variant | C11A, E79S, W85H, Q111E, | NUC |
| | | L186Y, L280A, P312N | |
| 329 | Lipase variant | L17P, K40M, E79S, N84T, | PRT |
| | | L186Y, A202N, P309C | |
| 330 | Lipase variant | L17P, K40M, E79S, N84T, | NUC |
| | | L186Y, A202N, P309C | |
| 331 | Lipase variant | C11A, Q31W, E79S, L186Y, | PRT |
| | | L280A, D301A, P309C | |
| 332 | Lipase variant | C11A, Q31W, E79S, L186Y, | NUC |
| | | L280A, D301A, P309C | |
| 333 | Lipase variant | K8E, K40M, E79S, L186W, | PRT |
| | | A202N, D301A, P312N | |
| 334 | Lipase variant | K8E, K40M, E79S, L186W, | NUC |
| | | A202N, D301A, P312N | |
| 335 | Lipase variant | M3Q, C11A, L186W, A202N, | PRT |
| | | L280A, P309C, P312N | |
| 336 | Lipase variant | M3Q, C11A, L186W, A202N, | NUC |
| | | L280A, P309C, P312N | |
| 337 | Lipase variant | L17P, K40M, L186W, F192A, | PRT |
| | | A202N, L280A, P309C | |
| 338 | Lipase variant | L17P, K40M, L186W, F192A, | NUC |
| | | A202N, L280A, P309C | |
| 339 | Lipase variant | K8E, L17P, N29W, K40M, | PRT |
| | | E79S, L186W, F192A, L280A | |
| 340 | Lipase variant | K8E, L17P, N29W, K40M, | NUC |
| | | E79S, L186W, F192A, L280A | |
| 341 | Lipase variant | S4P, N29H, N84T, Q111E, | PRT |
| | | L186Y, A202N, L280A, D301A | |
| 342 | Lipase variant | S4P, N29H, N84T, Q111E, | NUC |
| | | L186Y, A202N, L280A, D301A | |
| 343 | Lipase variant | L17P, K40M, E79S, Q111E, | PRT |
| | | F192A, L280A, P309C, P312N | |
| 344 | Lipase variant | L17P, K40M, E79S, Q111E, | NUC |
| | | F192A, L280A, P309C, P312N | |
| 345 | Lipase variant | M3Q, L17P, V18C, L186Y, | PRT |
| | | A202N, L280A, P309C, P312N | |
| 346 | Lipase variant | M3Q, L17P, V18C, L186Y, | NUC |
| | | A202N, L280A, P309C, P312N | |
| 347 | Lipase variant | M3Q, S4P, K8E, C11A, E79S, | PRT |
| | | W85H, L186Y, F192A, D301A | |
| 348 | Lipase variant | M3Q, S4P, K8E, C11A, E79S, | NUC |
| | | W85H, L186Y, F192A, D301A | |
| 349 | Lipase variant | K8E, E79S, W85H, Q111E, | PRT |
| | | L186Y, F192A, A202N, | |
| | | L280A, P309C | |
| 350 | Lipase variant | K8E, E79S, W85H, Q111E, | NUC |
| | | L186Y, F192A, A202N, | |
| | | L280A, P309C | |
| 351 | Lipase variant | K8E, K40M, E79S, Q111E, | PRT |
| | | L186W, L280A, D301A | |
| 352 | Lipase variant | K8E, K40M, E79S, Q111E, | NUC |
| | | L186W, L280A, D301A | |
| 353 | Lipase variant | L17P, E79S, L186Y, F192A, | PRT |
| | | A202N, L280A, D301A | |
| 354 | Lipase variant | L17P, E79S, L186Y, F192A, | NUC |
| | | A202N, L280A, D301A | |
| 355 | Lipase variant | Q31W, E79S, Q111E, L186W, | PRT |
| | | A202N, L280A, D301A | |
| 356 | Lipase variant | Q31W, E79S, Q111E, L186W, | NUC |
| | | A202N, L280A, D301A | |
| 357 | Lipase variant | L17P, K40M, E79S, Q111E, | PRT |
| | | L186W, F192A, D301A | |
| 358 | Lipase variant | L17P, K40M, E79S, Q111E, | NUC |
| | | L186W, F192A, D301A | |
| 359 | Lipase variant | C11A, K40M, E79S, Q111E, | PRT |
| | | L186W, F192A, D301A | |
| 360 | Lipase variant | C11A, K40M, E79S, Q111E, | NUC |
| | | L186W, F192A, D301A | |
| 361 | Lipase variant | L17P, V18A, K40M, Q111E, | PRT |
| | | L186Y, A202N, L280A | |
| 362 | Lipase variant | L17P, V18A, K40M, Q111E, | NUC |
| | | L186Y, A202N, L280A | |
| 363 | Lipase variant | M3Q, L17P, E79S, L186W, | PRT |
| | | A202N, D301A, P309C | |
| 364 | Lipase variant | M3Q, L17P, E79S, L186W, | NUC |
| | | A202N, D301A, P309C | |
| 365 | Lipase variant | M3Q, S4P, C11A, L17P, | PRT |
| | | E79S, Q111E, L186Y | |
| 366 | Lipase variant | M3Q, S4P, C11A, L17P, | NUC |
| | | E79S, Q111E, L186Y | |
| 367 | Lipase variant | K40M, E79S, Q111E, L186Y, | PRT |
| | | A202N, L280A, P309C, P312N | |
| 368 | Lipase variant | K40M, E79S, Q111E, L186Y, | NUC |
| | | A202N, L280A, P309C, P312N | |
| 369 | Lipase variant | C11A, N29W, Q31W, E79S, | PRT |
| | | L186Y, A202N, L280A, D301A | |
| 370 | Lipase variant | C11A, N29W, Q31W, E79S, | NUC |
| | | L186Y, A202N, L280A, D301A | |
| 371 | Lipase variant | L17P, K40M, E79S, Q111E, | PRT |
| | | L186W, F192A, L280A, P309C | |
| 372 | Lipase variant | L17P, K40M, E79S, Q111E, | NUC |
| | | L186W, F192A, L280A, P309C | |
| 373 | Lipase variant | L17P, N29H, Q31W, K40M, | PRT |
| | | E79S, Q111E, L186Y, L280A | |
| 374 | Lipase variant | L17P, N29H, Q31W, K40M, | NUC |
| | | E79S, Q111E, L186Y, L280A | |
| 375 | Lipase variant | K8E, K40M, E79S, Q111E, | PRT |
| | | L186W, A202N, L280A, P309C | |
| 376 | Lipase variant | K8E, K40M, E79S, Q111E, | NUC |
| | | L186W, A202N, L280A, P309C | |
| 377 | Lipase variant | S4P, K40M, E79S, Q111E, | PRT |
| | | L186Y, A202N, L280A, P309C | |
| 378 | Lipase variant | S4P, K40M, E79S, Q111E, | NUC |
| | | L186Y, A202N, L280A, P309C | |
| 379 | Lipase variant | M3Q, L17P, N29W, Q31W, | PRT |
| | | Q111E, L186Y, A202N, | |
| | | L280A, P309C | |
| 380 | Lipase variant | M3Q, L17P, N29W, Q31W, | NUC |
| | | Q111E, L186Y, A202N, | |
| | | L280A, P309C | |
| 381 | Lipase variant | L17P, K40M, E79S, L186Y, | PRT |
| | | A202N, L280A, D301A | |
| 382 | Lipase variant | L17P, K40M, E79S, L186Y, | NUC |
| | | A202N, L280A, D301A | |
| 383 | Lipase variant | M3Q, L17P, E79S, Q111E, | PRT |
| | | L186W, F192A, A202N, | |
| | | L280A | |
| 384 | Lipase variant | M3Q, L17P, E79S, Q111E, | NUC |
| | | L186W, F192A, A202N, | |
| | | L280A | |
| 385 | Lipase variant | M3Q, L17P, V18A, E79S, | PRT |
| | | Q111E, L186Y, L280A, | |
| | | D301A, P312N | |
| 386 | Lipase variant | M3Q, L17P, V18A, E79S, | NUC |
| | | Q111E, L186Y, L280A, | |
| | | D301A, P312N | |
| 387 | Lipase variant | M3Q, L17P, V18A, K40M, | PRT |
| | | E79S, N84T, L186Y, A202N, | |
| | | L280A | |
| 388 | Lipase variant | M3Q, L17P, V18A, K40M, | NUC |
| | | E79S, N84T, L186Y, A202N, | |
| | | L280A | |
| 389 | Lipase variant | L17P, K40M, E79S, N84T, | PRT |
| | | Q111E, L186Y, A202N, | |
| | | L280A, D301A | |
| 390 | Lipase variant | L17P, K40M, E79S, N84T, | NUC |
| | | Q111E, L186Y, A202N, | |
| | | L280A, D301A | |
| 391 | Lipase variant | M3Q, S4P, L17P, K40M, | PRT |
| | | E79S, L186Y, A202N, | |
| | | L280A, D301A | |
| 392 | Lipase variant | M3Q, S4P, L17P, K40M, | NUC |
| | | E79S, L186Y, A202N, | |
| | | L280A, D301A | |
| 393 | Lipase variant | N29W, Q31W, L186W, P312N | PRT |
| 394 | Lipase variant | N29W, Q31W, L186W, P312N | NUC |
| 395 | Lipase variant | L186Y, F192A, P309C, P312N | PRT |
| 396 | Lipase variant | L186Y, F192A, P309C, P312N | NUC |
| 397 | Lipase variant | N29W, Q31W, W85H, L186Y, | PRT |
| | | P312N | |
| 398 | Lipase variant | N29W, Q31W, W85H, L186Y, | NUC |
| | | P312N | |
| 399 | Lipase variant | K40M, L42D, E79S, N84T, | PRT |
| | | L186Y, F192A, D301A | |
| 400 | Lipase variant | K40M, L42D, E79S, N84T, | NUC |
| | | L186Y, F192A, D301A | |
| 401 | Lipase variant | E79S, N84T, L186Y, F192V, | PRT |
| | | Q217M, D301A | |
| 402 | Lipase variant | E79S, N84T, L186Y, F192V, | NUC |
| | | Q217M, D301A | |

### EXAMPLES

Terrific broth culture (TB) media were prepared in demineralized water with the use of 47.6 g/l of granulated medium and 4 ml/L glycerol and sterilized at 121° C. for 20 minutes.

### Example 1

### Cloning of Lipase Variants

Nucleotide sequences encoding lipase variants as described herein can be synthesized as known in the art, e.g. as offered by respective service providers such as Eurofins Genomics GmbH (Eurofins Genomics GmbH, Anzinger Str. 7a, 85560 Ebersberg, Germany). In brief, nucleic acid sequences of wild-type lipase (SEQ ID NO. 2) or related variants as described herein were cloned into an expression vector based on the vector pKA81a. Genetic elements were introduced into the modified pKA81a vector by means of commonly known methods. For expression of wild-type lipase and lipase variants respectively, the expression vectors were introduced into electro-competent Escherichia coli MG1655 cells.

### Generation of Enzyme Variants

Nucleotide substitutions (replacements) were introduced into the nucleic acid parent sequences, e.g. to obtain an amino acid exchange into other amino acids. Several Molecular Biological methods can be used to achieve these replacements. A useful method for preparing a mutated nucleic acid and the corresponding mutated protein according to the invention is site-directed mutagenesis on codons encoding one or more amino acids, which are selected in advance. The methods for obtaining these site-directed mutations are well known to the skilled person and widely described in the literature (in particular: Directed Mutagenesis: A Practical Approach, 1991, Edited by M.J. McPHERSON, IRL PRESS), or are methods for which it is possible to employ commercial kits (for example the QUIKCHANGE™ lightening mutagenesis kit from Qiagen or Stratagene). After site-directed mutagenesis, nucleic acids were transformed into the Escherichia coli MG1655 cells.

Transformed cells were tested in appropriate biotransformation reactions in order to determine product yield and selectivity. Appropriate biotransformation reactions are described below, see e.g. Example 2. Sequence verification was performed as known in the art.

Glycerol stocks of the E. coli cultures transformed with the respective expression plasmids were prepared by adding one volume of 40% glycerol solution to one volume of E. coli culture.

For isolation of single bacterial colonies, appropriate dilutions of E. coli cultures were plated onto LB-Agar plates containing suitable concentrations of kanamycin and incubated at 37° C. until single colonies were obtained.

### Example 2

Conversion of Racemate DMAI into DMAI Carbamate Using Wild-Type Lipase or Variants Thereof

### Cultivation

For screening purposes Escherichia coli MG1655 was used as host for expression plasmids. For pre-cultures, sterilized 2-mL 96-well deep-well plates (0030501306, Eppendorf, Hamburg, Germany) were filled with 590 μL TB-medium (TB, Fisher Bioreagents, 22711022) supplemented with 50 μg/ml kanamycin (Kanamycin solution, K0254, Sigma-Aldrich, St. Louis, MO, USA), inoculated with 10 μL of glycerol stocks of the respective variants or alternatively 590 μL TB-medium were inoculated with cell material from Agar plate colonies. Pre-cultures were incubated for 17 hours at 37° C. and 250 rpm in a climo shaker ISF1-X (Kühner AG, Birsfelden, Switzerland).

For expression cultures, sterilized 2-mL 96-well deep-well plates (0030501306, Eppendorf, Hamburg, Germany) were filled with 510 μL TB expression medium supplemented with kanamycin (50 mg/L); expression cultures were inoculated with 30 μL of pre-cultures. The expression culture plates were incubated at 37° C. and 250 rpm in a climo shaker. After 4 h incubation 60 μL of 10 mM IPTG diluted in expression medium (supplemented with 50 mg/L kanamycin) was added per well to induce enzyme expression (resulting in a final concentration of 1 mM IPTG). The expression culture plates were subsequently incubated for 20 hours at 28° C. in a shaking incubator.

Cells were harvested by centrifugation of the expression cultures for 15 minutes at 4° C. and 2500×g. The culture supernatant was discarded, and the remaining cell pellets were resuspended in 200 μL of PBS (Gibco PBS, no Mg²⁺ no Ca²⁺). Subsequently the resuspended culture was lyophilized for 24 h. The deep-well plates were stored at 4° C. until use.

### Biotransformation

Deep-well plates containing the cell suspensions were equilibrated to room temperature.

Biotransformation reactions were started by addition of 13.2 μL of racemic 2,6-dimethyl-1-indanylamine (DMAI) and 186.8 μl of dimethylcarbonate to the lyophilized culture and incubation occurred for 8 h-22 h at 70° C. or 80° C. in a shaking incubator at 250 rpm. Reactions were stopped by adding 600 μl acetonitrile. Afterwards the plates were centrifuged for 15 min at 4° C. and 2500×g. From each well 100 μL of the supernatant was transferred to a 96-well plate. The plates were stored at −20° C. or directly submitted to HPLC analysis.

### Analytical HPLC Method

Analytical HPLC was performed with the following setup:

Instrument: Agilent Technologies 1290 Infinity; Column: Poroshell, 100×4.6 mm, 2.7 μm; Eluent A: Water (+0.001% formic acid); Eluent B: acetonitrile; Flow: isocratic (10% Eluent A/90% Eluent B), flow rate: 1.4 mL/min; Temperature: 25° C.; sample injection volume: 1 μL; detection: Absorption at 210 nm.

1R,2S DMAI carbamate (dissolved in acetonitrile) was used as reference substance and as a standard for quantification. Appropriate dilutions were prepared covering the range up to the maximum concentration used in the biotransformation. Samples were analyzed for acylated or carboxylated product isomers and non-acylated and non-carboxylated substrate. The concentration of the respective compounds in samples was calculated by comparing the obtained peak areas with those of the respective compound standards.

### Example 3

Conversion of Racemic 2,6-dimethyl-1-indanylamine (DMAI) into methyl-[(1R,2S)-2,6-dimethyl-2,3-dihydro-1H-inden-1-yl]carbamate Yield Using Lipase Variants

Cultivation, biotransformation and HPLC analysis were performed as described in Example 2. Biotransformations were performed for 22 h at 70° C. Glycerol stocks were used as inoculum for cultivation. Results for methyl-[(1R,2S)-2,6-dimethyl-2,3-dihydro-1H-inden-1-yl]carbamat product yield and the diastereomeric ratio (d.r.) for methyl-[(1R,2S)-2,6-dimethyl-2,3-dihydro-1H-inden-1-yl]carbamate are shown in Table 5 and 6. The relative methyl-[(1R,2S)-2,6-dimethyl-2,3-dihydro-1H-inden-1-yl]carbamate yield is defined as methyl-[(1R,2S)-2,6-dimethyl-2,3-dihydro-1H-inden-1-yl]carbamate yield of variant divided by methyl-[(1R,2S)-2,6-dimethyl-2,3-dihydro-1H-inden-1-yl]carbamate of the wild-type lipase. d.r. is defined as yield methyl-[(1R,2S)-2,6-dimethyl-2,3-dihydro-1H-inden-1-yl]carbamate divided by the sum of methyl-[(1R,2S)-2,6-dimethyl-2,3-dihydro-1H-inden-1-yl]carbamate, methyl-[(1S,2R)-2,6-dimethyl-2,3-dihydro-1H-inden-1-yl]carbamate, methyl-[(1R,2R)-2,6-dimethyl-2,3-dihydro-1H-inden-1-yl]carbamate and methyl-[(1S,2S)-2,6-dimethyl-2,3-dihydro-1H-inden-1-yl]carbamate in percent.

| | Relative |
|---|---|
| | 1R,2S |
| | DMAI |
| | carbamate |
| WT/Variant | yield [%] |
| WT | 100.0 |
| L186F | 1057.4 |
| L280E | 828.2 |
| L280S | 782.6 |
| L280K | 694.6 |
| P312N | 692.7 |
| P312F | 647.1 |
| L186W | 633.7 |
| M3L | 631.4 |
| N29Y | 584.7 |
| L17P | 546.9 |
| S4P | 499.0 |
| L280D | 493.9 |
| V18A | 472.2 |
| M3Q | 467.8 |
| P312D | 442.7 |
| N29W | 422.6 |
| L186Y | 403.0 |
| V18T | 391.5 |
| L186E | 390.2 |
| A202Q | 383.2 |
| D301A | 381.1 |
| P309C | 379.9 |
| L186D | 352.4 |
| Q31W | 329.3 |
| A202N | 322.3 |
| Q111E | 309.3 |
| L186Q | 309.2 |
| L186T | 301.8 |
| M3C | 280.9 |
| W85H | 272.3 |
| N29H | 261.6 |
| K8E | 260.5 |
| L186H | 244.4 |
| E79I | 217.5 |
| E79W | 214.4 |
| L17T | 199.8 |
| V18C | 196.4 |
| L186P | 194.2 |
| P312Q | 193.8 |
| K40M | 190.3 |
| P312K | 188.4 |
| L186C | 185.7 |
| L280A | 181.4 |
| S4L | 164.2 |
| V18S | 162.1 |
| L186K | 156.4 |
| L186S | 155.4 |

Table Key for an efficient production process is a high selectivity of the respective enzyme variant in order to generate almost enantiopure methyl-[(1R,2S)-2,6-dimethyl-2,3-dihydro-1H-inden-1-yl]carbamate. Wild-type lipase (SEQ ID NO. 1) showed a methyl-[(1R,2S)-2,6-dimethyl-2,3-dihydro-1H-inden-1-yl]carbamate product yield of 0.96 mg/ml and a d.r. of 61.7%.

TABLE 5
Relative IR,2S DMAI carbamate yield for lipase variants
with improved 1R,2S DMAI carbamate product yield.

| | Relative |
|---|---|
| | 1R,2S |
| | DMAI |
| | carbamate |
| WT/Variant | yield [%] |
| WT | 100.0 |
| L186F | 1057.4 |
| L280E | 828.2 |
| L280S | 782.6 |
| L280K | 694.6 |
| P312N | 692.7 |
| P312F | 647.1 |
| L186W | 633.7 |
| M3L | 631.4 |
| N29Y | 584.7 |
| L17P | 546.9 |
| S4P | 499.0 |
| L280D | 493.9 |
| V18A | 472.2 |
| M3Q | 467.8 |
| P312D | 442.7 |
| N29W | 422.6 |
| L186Y | 403.0 |
| V18T | 391.5 |
| L186E | 390.2 |
| A202Q | 383.2 |
| D301A | 381.1 |
| P309C | 379.9 |
| L186D | 352.4 |
| Q31W | 329.3 |
| A202N | 322.3 |
| Q111E | 309.3 |
| L186Q | 309.2 |
| L186T | 301.8 |
| M3C | 280.9 |
| W85H | 272.3 |
| N29H | 261.6 |
| K8E | 260.5 |
| L186H | 244.4 |
| E79I | 217.5 |
| E79W | 214.4 |
| L17T | 199.8 |
| V18C | 196.4 |
| L186P | 194.2 |
| P312Q | 193.8 |
| K40M | 190.3 |
| P312K | 188.4 |
| L186C | 185.7 |
| L280A | 181.4 |
| S4L | 164.2 |
| V18S | 162.1 |
| L186K | 156.4 |
| L186S | 155.4 |

TABLE 6
Lipase variants with improved diastereomeric ratio (d.r.).

| | Relative |
|---|---|
| | DMAI |
| | carbamate |
| WT/Variant | yield [%] |
| WT | 61.7 |
| L186F | 88.1 |
| P309C | 87.2 |
| L186W | 81.9 |
| L186Y | 79.6 |
| P312N | 79.3 |
| L186Q | 78.9 |
| L17P | 78.8 |
| L280S | 78.7 |
| M3L | 78.6 |
| L280K | 78.5 |
| L280E | 78.3 |
| N29Y | 78.3 |
| V18A | 78.2 |
| M3Q | 78.0 |
| S4P | 77.9 |
| L186N | 77.9 |
| L186H | 77.8 |
| N29W | 77.3 |
| L186D | 77.2 |
| P312F | 76.7 |
| L186E | 76.7 |
| E79W | 76.4 |
| P312D | 76.2 |
| L280D | 76.1 |
| L186C | 76.1 |
| D301A | 76.1 |
| L186T | 76.0 |
| Q31W | 75.6 |
| A202Q | 75.4 |
| Q111E | 75.3 |
| V18T | 74.8 |
| L186I | 74.4 |
| L186V | 74.3 |
| A202N | 73.8 |
| W85H | 73.4 |
| K8E | 73.0 |
| M3C | 72.7 |
| N29H | 72.7 |
| L186P | 72.5 |
| E79I | 71.8 |
| L186S | 71.6 |
| L17T | 70.3 |
| V18C | 69.8 |
| E79T | 69.6 |
| L280A | 69.1 |
| P312K | 69.1 |
| K40M | 68.9 |
| P312Q | 68.1 |

### Example 4

Conversion of racemate 2,6-Dimethyl-1-indanamine into methyl-[(1R,2S)-2,6-dimethyl-2,3-dihydro-1H-inden-1-yl]carbamate using lipase variants based on wild-type lipase. Cultivation, biotransformation and HPLC analysis were performed as described in Example 2.

Biotransformations were performed for 8 h at 80° C. Glycerol stocks were used as inoculum for cultivation. Results for methyl-[(1R,2S)-2,6-dimethyl-2,3-dihydro-1H-inden-1-yl]carbamate product yield and the diastereomeric ratio (d.r.) for methyl-[(1R,2S)-2,6-dimethyl-2,3-dihydro-1H-inden-1-yl]carbamate are shown in Table 7 and Table 8.

Wild type lipase (SEQ ID NO. 1) showed a product yield of 2.04 mg/ml and a d.r. of 64.6%.

TABLE 7
Relative methyl-[(1R,2S)-2,6-dimethyl-2,3-dihydro-
1H-inden-1-yl]carbamate yield for lipase variants with
improved 1R,2S DMAI carbamate product yield.

| | Relative |
|---|---|
| | DMAI |
| | carbamate |
| WT/Variant | yield [%] |
| WT | 100.0 |
| L17P, N29W, E79S, L186Y, A202N, P312N | 1229.2 |
| E79S, N84T, L186W, A202N, L280A, D301A | 1050.1 |
| M3Q, E79S, L186Y, P312N | 988.0 |
| K8E, K40M, E79S, N84T, L186Y, P312N | 971.4 |
| C11A, E79S, W85H, Q111E, L186Y, L280A, P312N | 806.0 |
| L17P, E79S, L186W, A202N | 787.2 |
| E79S, L186W, P309C, P312N | 756.5 |
| L17P, E79S, Q111E, L186W, P309C | 734.2 |
| K40M, E79S, L186Y, P312N | 723.2 |
| E79S, Q111E, L186W, L280A, D301A | 686.3 |
| L17P, K40M, E79S, N84T, Q111E, L186Y, A202N, | 675.6 |
| L280A, D301A | |
| Q31W, N84T, Q111E, L186Y, L280A, P312N | 675.0 |
| L17P, E79S, N84T, L186Y, L280A, P309C | 647.3 |
| L17P, V18C, E79S, L186W, L280A | 643.1 |
| M3Q, L17P, V18A, E79S, Q111E, L186Y, L280A, D301A, | 636.1 |
| P312N | |
| S4P, N29W, E79S, L186W, A202N, L280A, P309C | 612.7 |
| M3Q, L17P, E79S, Q111E, L186W, F192A, A202N, L280A | 543.8 |
| K40M, E79S, Q111E, L186Y, A202N, L280A, P309C, | 534.5 |
| P312N | |
| C11A, L186Y, P312N | 533.6 |
| L17P, K40M, E79S, N84T, L186Y, A202N, P309C | 528.9 |
| S4P, E79S, L186W, A202N, P309C | 525.3 |
| L186Y, L280A, P312N | 511.9 |
| K8E, K40M, E79S, Q111E, L186W, L280A, D301A | 510.6 |
| L17P, L186Y, P312N | 504.7 |
| M3Q, L17P, N29H, E79S, L186Y, D301A | 463.1 |
| L17P, E79S, L186Y, F192A, A202N, L280A, D301A | 454.4 |
| N29W, E79S, Q111E, L186W, L280A, P309C, P312N | 452.7 |
| Q31W, E79S, Q111E, L186W, A202N, L280A, D301A | 445.2 |
| C11A, N29W, Q31W, E79S, L186Y, A202N, L280A, D301A | 443.0 |
| L17P, K40M, E79S, Q111E, L186W, F192A, L280A, P309C | 434.8 |
| N29H, K40M, E79S, L186W, D301A | 414.8 |
| V18A, K40M, N84T, Q111E, L186Y, L280A, P309C, P312N | 405.5 |
| Q31W, E79S, L186W, A202N, P309C | 393.0 |
| E79S, L186W, L280A, P309C, P312N | 382.2 |
| N29W, Q31W, W85H, L186Y, P312N | 375.6 |
| Q31W, L186Y, F192A, L280A, D301A, P312N | 373.9 |
| M3Q, S4P, L17P, K40M, E79S, L186Y, A202N, L280A, | 368.1 |
| D301A | |
| S4P, K8E, N29W, E79S, Q111E, L186W | 359.6 |
| N29H, E79S, L186W, A202N, L280A | 356.6 |
| L186Y, F192A, A202N, P312N | 344.7 |
| K8E, C11A, L186W, P312N | 343.3 |
| L17P, K40M, E79S, Q111E, L186W, F192A, D301A | 343.1 |
| C11A, K40M, E79S, Q111E, L186W, F192A, D301A | 340.4 |
| L17P, Q111E, L186Y, A202N, L280A, P312N | 338.1 |
| K8E, C11A, K40M, E79S, W85H, L186W, P309C | 337.8 |
| N29H, E79S, Q111E, L186W, L280A | 337.0 |
| L17P, V18A, L186W, L280A, P309C, P312N | 331.8 |
| K40M, E79S, L186Y, D301A | 318.3 |
| E79S, N84T, Q111E, L186Y, P309C, P312N | 318.2 |
| S4P, C11A, V18A, K40M, N84T, L186Y, D301A, P312N | 317.8 |
| L17P, N29H, Q31W, K40M, E79S, Q111E, L186Y, L280A | 304.2 |
| E79S, L186W, A202N, L280A | 303.9 |
| L17P, N29H, E79S, W85H, L186W, L280A | 302.3 |
| K8E, L17P, N29W, K40M, E79S, L186W, F192A, L280A | 299.4 |
| L17P, K40M, E79S, L186Y, A202N, L280A, D301A | 297.0 |
| K8E, L186Y, L280A, D301A | 288.7 |
| M3Q, S4P, K8E, N84T, L186W, P312N | 284.2 |
| L17P, E79S, L186Y, F192A, A202N, P309C | 270.0 |
| M3Q, C11A, L186Y, A202N, L280A, P312N | 267.3 |
| Q31W, E79S, L186W, F192A, D301A | 264.7 |
| C11A, Q31W, E79S, L186Y, L280A, D301A, P309C | 262.4 |
| M3Q, L17P, K40M, E79S, L186Y | 260.8 |
| N29W, Q31W, L186W, P312N | 260.5 |
| L17P, K40M, Q111E, L186W, L280A | 252.5 |
| N29H, E79S, L186W, A202N, D301A | 251.8 |
| E79S, L186Y, P309C, P312N | 244.1 |
| C11A, E79S, L186Y, A202N, D301A | 236.2 |
| L186W, L280A, P312N | 235.0 |
| L17P, K40M, E79S, Q111E, L186Y | 233.9 |
| L17P, E79S, L186Y, P309C | 233.1 |
| L17P, E79S, Q111E, L186Y, F192A, D301A | 229.4 |
| V18A, K40M, E79S, L186W, F192A, D301A | 226.3 |
| L17P, N29H, Q31W, E79S, L186W, D301A | 225.9 |
| L186Y, F192A, L280A, D301A | 224.2 |
| L186Y, A202N, D301A | 222.4 |
| L17P, L186W, A202N | 221.3 |
| E79S, N84T, L186W, P309C | 215.8 |
| L17P, V18A, K40M, Q111E, L186Y, A202N, L280A | 213.5 |
| Q31W, E79S, N84T, L186W, A202N | 210.2 |
| C11A, L186Y, L280A, D301A, P309C | 209.4 |
| K8E, K40M, E79S, Q111E, L186W, A202N, L280A, P309C | 206.8 |
| K8E, E79S, L186Y, L280A, D301A | 206.6 |
| S4P, K8E, C11A, L186Y, F192A, A202N, L280A, P309C | 205.7 |
| S4P, N29H, N84T, Q111E, L186Y, A202N, L280A, D301A | 205.6 |
| V18C, K40M, N84T, L186W, P312N | 205.4 |
| L186Y, A202N, L280A, P312N | 197.2 |
| M3Q, L17P, E79S, L186W, A202N, D301A, P309C | 195.5 |
| M3Q, L17P, V18A, K40M, E79S, N84T, L186Y, A202N, | 194.0 |
| L280A | |
| N29H, L186W, L280A | 190.6 |
| L186Y, F192A, P309C, P312N | 189.2 |
| M3Q, S4P, C11A, L17P, E79S, Q111E, L186Y | 189.0 |
| C11A, N29W, E79S, N84T, W85H, L186Y, P309C | 185.7 |
| E79S, N84T, L186Y, F192A, L280A | 181.4 |
| C11A, L17P, K40M, E79S, L186Y | 178.8 |
| M3Q, S4P, K8E, C11A, E79S, W85H, L186Y, F192A, | 177.4 |
| D301A | |
| K8E, K40M, Q111E, L186W, L280A, P309C | 177.1 |
| S4P, N29H, L186Y, A202N, P312N | 174.4 |
| S4P, K40M, E79S, Q111E, L186Y, A202N, L280A, P309C | 174.4 |
| E79S, N84T, L186W, F192A, L280A | 173.8 |
| L17P, N29W, Q111E, L186W, A202N | 173.1 |
| K8E, K40M, E79S, L186W, A202N, D301A, P312N | 171.8 |
| L17P, L186Y, P309C | 169.2 |
| N29H, N84T, L186Y, A202N, L280A, P309C, P312N | 167.0 |
| V18A, L186Y, F192A, D301A, P312N | 165.0 |
| N29H, Q111E, L186W, P309C, P312N | 162.9 |
| S4P, L186W, A202N | 161.8 |
| K40M, Q111E, L186Y, L280A, D301A | 158.7 |
| N29H, L186Y, F192A, L280A, D301A, P312N | 158.5 |
| S4P, L17P, N29W, L186Y, D301A | 150.8 |
| N29H, E79S, L186W, F192A, A202N | 150.2 |
| K40M, L186W, L280A, P309C | 149.7 |
| N29H, Q31W, E79S, N84T, W85H, L186Y, F192A, | 148.7 |
| L280A, P309C | |
| M3Q, K8E, L17P, L186Y, D301A, P312N | 146.8 |
| K8E, C11A, N29H, Q31W, L186Y, D301A | 142.8 |
| L17P, Q111E, L186Y, D301A | 138.4 |
| E79S, L186Y | 136.7 |
| M3Q, L17P, N29W, Q31W, Q111E, L186Y, A202N, | 134.2 |
| L280A, P309C | |

TABLE 8
Selection of variants with an improvement in d.r..

| WT/Variant | d.r. |
|---|---|
| WT | 64.6 |
| M3Q, L17P, N29H, E79S, L186Y, D301A | 91.9 |
| C11A, N29W, Q31W, E79S, L186Y, A202N, L280A, D301A | 91.8 |
| L17P, K40M, E79S, N84T, L186Y, A202N, P309C | 90.3 |
| M3Q, L17P, V18A, K40M, E79S, N84T, L186Y, A202N, L280A | 90.0 |
| L17P, E79S, N84T, L186Y, L280A, P309C | 90.0 |
| L17P, N29H, Q31W, K40M, E79S, Q111E, L186Y, L280A | 90.0 |
| M3Q, S4P, L17P, K40M, E79S, L186Y, A202N, L280A, D301A | 89.2 |
| E79S, L186Y | 88.7 |
| C11A, E79S, W85H, Q111E, L186Y, L280A, P312N | 88.0 |
| K8E, K40M, E79S, N84T, L186Y, P312N | 87.6 |
| L17P, K40M, E79S, L186Y, A202N, L280A, D301A | 87.3 |
| K40M, E79S, L186Y, P312N | 87.2 |
| M3Q, E79S, L186Y, P312N | 87.0 |
| Q31W, E79S, Q111E, L186W, A202N, L280A, D301A | 86.9 |
| K40M, E79S, L186Y, D301A | 86.8 |
| L17P, K40M, E79S, N84T, Q111E, L186Y, A202N, L280A, | 86.7 |
| D301A | |
| C11A, Q31W, E79S, L186Y, L280A, D301A, P309C | 86.7 |
| M3Q, S4P, C11A, L17P, E79S, Q111E, L186Y | 86.5 |
| L17P, E79S, Q111E, L186W, P309C | 86.3 |
| C11A, L17P, K40M, E79S, L186Y | 86.3 |
| N29H, K40M, E79S, L186W, D301A | 86.2 |
| C11A, K40M, E79S, Q111E, L186W, F192A, D301A | 86.1 |
| L17P, K40M, E79S, Q111E, L186Y | 85.9 |
| L17P, E79S, L186Y, F192A, A202N, L280A, D301A | 85.8 |
| M3Q, L17P, V18A, E79S, Q111E, L186Y, L280A, D301A, | 85.5 |
| P312N | |
| L17P, K40M, E79S, Q111E, L186W, F192A, D301A | 85.5 |
| L17P, E79S, Q111E, L186Y, F192A, D301A | 85.3 |
| M3Q, L17P, E79S, Q111E, L186W, F192A, A202N, L280A | 85.3 |
| Q31W, E79S, L186W, F192A, D301A | 85.2 |
| E79S, L186Y, P309C, P312N | 85.1 |
| E79S, N84T, Q111E, L186Y, P309C, P312N | 85.1 |
| V18A, K40M, E79S, L186W, F192A, D301A | 84.8 |
| L17P, E79S, L186W, A202N | 84.7 |
| K8E, E79S, L186Y, L280A, D301A | 84.7 |
| E79S, L186W, P309C, P312N | 84.6 |
| K40M, E79S, Q111E, L186Y, A202N, L280A, P309C, P312N | 84.5 |
| L17P, E79S, L186Y, F192A, A202N, P309C | 84.4 |
| L17P, N29W, E79S, L186Y, A202N, P312N | 84.4 |
| L17P, K40M, E79S, Q111E, L186W, F192A, L280A, P309C | 84.3 |
| E79S, W85H, L186Y, F192A, D301A | 84.2 |
| E79S, N84T, L186Y, F192A, L280A | 84.2 |
| M3Q, L17P, K40M, E79S, L186Y | 84.1 |
| S4P, E79S, L186W, A202N, P309C | 84.1 |
| C11A, E79S, L186Y, A202N, D301A | 84.1 |
| Q31W, E79S, L186W, A202N, P309C | 84.1 |
| L17P, E79S, L186Y, A202N | 84.1 |
| L17P, V18C, E79S, L186W, L280A | 84.1 |
| N29H, E79S, Q111E, L186W, L280A | 84.0 |
| M3Q, L17P, E79S, L186W, A202N, D301A, P309C | 84.0 |
| S4P, K40M, E79S, Q111E, L186Y, A202N, L280A, P309C | 83.7 |
| L17P, E79S, L186Y, P309C | 83.7 |
| S4P, N29W, E79S, L186W, A202N, L280A, P309C | 83.7 |
| S4P, K8E, N29W, E79S, Q111E, L186W | 83.4 |
| S4P, N29H, Q31W, E79S, L280A, D301A | 82.8 |
| Q31W, E79S, N84T, L186W, A202N | 82.7 |
| K8E, L17P, N29W, K40M, E79S, L186W, F192A, L280A | 82.6 |
| N29W, E79S, Q111E, L186W, L280A, P309C, P312N | 82.6 |
| E79S, Q111E, L186W, L280A, D301A | 82.4 |
| K8E, C11A, K40M, E79S, W85H, L186W, P309C | 82.4 |
| L17P, N29H, Q31W, E79S, L186W, D301A | 82.3 |
| K8E, K40M, E79S, Q111E, L186W, L280A, D301A | 82.1 |
| E79S, L186W, A202N, L280A | 81.9 |
| C11A, N29W, E79S, N84T, W85H, L186Y, P309C | 81.7 |
| E79S, L186W, L280A, P309C, P312N | 81.5 |
| N29H, E79S, L186W, A202N, L280A | 81.0 |
| L17P, N29H, E79S, W85H, L186W, L280A | 80.6 |
| N29H, E79S, L186W, A202N, D301A | 80.5 |
| M3Q, E79S, L186Y, D301A | 80.4 |
| M3Q, S4P, K8E, C11A, E79S, W85H, L186Y, F192A, D301A | 80.0 |
| E79S, N84T, L186W, P309C | 79.7 |
| K8E, K40M, E79S, Q111E, L186W, A202N, L280A, P309C | 79.6 |
| L17P, N29H, E79S, L186Y, P309C | 79.4 |
| E79S, N84T, L186W, A202N, L280A, D301A | 79.0 |
| N29H, E79S, L186W, F192A, A202N | 78.9 |
| N29H, Q31W, E79S, N84T, W85H, L186Y, F192A, L280A, | 78.9 |
| P309C | |
| E79S, N84T, L186W, F192A, L280A | 77.9 |
| E79S, L186W, F192A, P312N | 77.4 |
| K8E, K40M, E79S, L186W, A202N, D301A, P312N | 77.3 |
| E79S, L186W, L280A, D301A | 77.3 |
| K8E, E79S, W85H, Q111E, L186Y, F192A, A202N, L280A, | 77.1 |
| P309C | |
| Q31W, N84T, Q111E, L186Y, L280A, P312N | 76.8 |
| N29W, Q31W, W85H, L186Y, P312N | 76.4 |
| S4P, C11A, V18A, K40M, N84T, L186Y, D301A, P312N | 76.2 |
| E79S, L186Y, D301A | 75.7 |
| L17P, V18A, K40M, Q111E, L186Y, A202N, L280A | 75.6 |
| S4P, N29H, N84T, Q111E, L186Y, A202N, L280A, D301A | 75.5 |
| C11A, L186Y, L280A, D301A, P309C | 75.5 |
| K8E, N29H, Q31W, E79S, L186W, D301A | 75.5 |
| C11A, N29W, E79S, L186Y | 75.4 |
| L17P, Q111E, L186Y, A202N, L280A, P312N | 75.2 |
| V18A, K40M, N84T, Q111E, L186Y, L280A, P309C, P312N | 75.2 |
| L186Y, A202N, L280A, P312N | 75.0 |
| L17P, K40M, Q111E, L186W, L280A | 74.2 |
| L186Y, A202N, D301A | 74.2 |
| N29H, Q31W, L186Y, L280A, D301A | 74.0 |
| M3Q, L17P, N29W, Q31W, Q111E, L186Y, A202N, L280A, | 73.8 |
| P309C | |
| Q31W, L186Y, F192A, L280A, D301A, P312N | 73.7 |
| L17P, L186Y, P312N | 73.7 |
| M3Q, S4P, N29H, K40M, L186Y, A202N, P309C | 73.7 |
| K8E, L186Y, L280A, D301A | 73.6 |
| N29H, N84T, L186Y, A202N, L280A, P309C, P312N | 73.6 |
| L186Y, L280A, P312N | 73.6 |
| L186Y, F192A, P309C, P312N | 73.2 |
| L186Y, F192A, L280A, D301A | 73.1 |
| L186Y, F192A, A202N, P312N | 73.1 |
| L17P, L186Y, P309C | 72.9 |
| M3Q, C11A, L186Y, A202N, L280A, P312N | 72.7 |
| N29W, L186Y, A202N, D301A | 72.6 |
| S4P, C11A, W85H, L186Y, A202N | 72.6 |
| S4P, L17P, N29W, L186Y, D301A | 72.4 |
| V18A, L186Y, F192A, D301A, P312N | 72.4 |
| N29W, Q31W, L186W, P312N | 72.4 |
| K8E, C11A, L186W, P312N | 72.3 |
| N29H, L186W, L280A | 72.2 |
| C11A, L186Y, P312N | 72.1 |
| L17P, N29H, E79S, L186Y | 72.1 |
| S4P, K8E, C11A, L186Y, F192A, A202N, L280A, P309C | 72.0 |
| L17P, K40M, E79S, Q111E, F192A, L280A, P309C, P312N | 72.0 |
| S4P, N29H, L186Y, A202N, P312N | 71.9 |
| K8E, C11A, N29H, Q31W, L186Y, D301A | 71.9 |
| K40M, Q111E, L186Y, L280A, D301A | 71.7 |
| M3Q, L17P, V18C, L186Y, A202N, L280A, P309C, P312N | 71.7 |
| N29H, L186Y, F192A, L280A, D301A, P312N | 71.6 |
| M3Q, K8E, L17P, L186Y, D301A, P312N | 71.5 |
| L17P, V18A, L186W, L280A, P309C, P312N | 71.3 |
| L17P, Q111E, L186Y, D301A | 71.2 |
| V18C, K40M, N84T, L186W, P312N | 71.1 |
| K40M, L186W, L280A, P309C | 71.0 |
| N29H, N84T, L186Y, A202N | 70.9 |
| K8E, K40M, Q111E, L186W, L280A, P309C | 70.7 |
| L186W, A202N, P312N | 70.7 |
| K8E, C11A, L186Y, D301A | 70.0 |
| L17P, L186Y, L280A, D301A, P309C | 70.0 |
| L186W, L280A, P312N | 69.9 |
| S4P, L186W, A202N | 69.8 |
| S4P, E79S, W85H, L186Y, L280A | 69.6 |
| L17P, L186W, A202N | 69.6 |
| M3Q, S4P, K8E, N84T, L186W, P312N | 69.2 |
| Q111E, L186W, A202N, L280A, P312N | 69.2 |
| N29H, Q111E, L186W, P309C, P312N | 69.0 |
| K8E, L17P, V18A, Q111E, L186W, P309C | 68.8 |
| L17P, N29W, Q111E, L186W, A202N | 68.8 |
| K8E, C11A, L186W, A202N, L280A, P309C | 68.5 |
| N29H, E79S, Q111E, L186Y, D301A | 68.1 |
| L17P, K40M, L186W, F192A, A202N, L280A, P309C | 67.9 |

1. A protein having lipase activity encoded by an amino acid sequence having at least 80% identity with the amino acid sequence as set forth in SEQ ID No. 1 apart from that said amino acid sequence having at least 80% sequence identity with the amino acid sequence as set forth in SEQ ID No. 1 comprises a modification selected from the group consisting of
i. the amino acid at position 186 is different from L;
ii. the amino acid at position 280 is different from L;
iii. the amino acid at position 312 is different from P;
iv. the amino acid at position 3 is different from M;
v. the amino acid at position 29 is different from N;
vi. the amino acid at position 17 is different from L;
vii. the amino acid at position 4 is different from S;
viii. the amino acid at position 18 is different from V;
ix. the amino acid at position 202 is different from A;
x. the amino acid at position 301 is different from D;
xi. the amino acid at position 309 is different from P;
xii. the amino acid at position 31 is different from Q;
xiii. the amino acid at position 111 is different from Q;
xiv. the amino acid at position 85 is different from W;
xv. the amino acid at position 8 is different from K;
xvi. the amino acid at position 79 is different from E; and
xvii. the amino acid at position 40 is different from K.

2. The protein according to claim 1, wherein the protein is selected from the group consisting of
a) proteins comprising the amino acid sequence as shown in SEQ ID No. 1 apart from that the amino acid at position 186 is different from L; and
b) proteins having an amino acid sequence having at least 80% identity with the amino acid sequence as set forth in a) given that the amino acid at position 186 is different from L.

3. The protein according to claim 2, wherein the amino acid at position 186 is F, W, Y, E, D, Q, T, H, P, C, K, S, N, I, or V.

4. The protein according to claim 3, wherein the protein has at least one further amino acid substitution selected from the group consisting of
(i) the amino acid at position 79 is different from E;
(ii) that the amino acid at position 202 is different from A;
(iii) the amino acid at position 280 is different from L;
(iv) the amino acid at position 301 is different from D;
(v) the amino acid at position 3 is different from M;
(VI) the amino acid at position 11 is different from C;
(vii) the amino acid at position 17 is different from L;
(viii) the amino acid at position 40 is different from K; and
(ix) the amino acid at position 111 is different from Q.

5. The protein according to claim 4, wherein the protein has at least two of the further amino acid substitutions selected from the group consisting of the sections (i), (ii), (iii), (iv), (v), (vi), (vii), (viii) and (ix) as defined in claim 4.

6. The protein according to claim 4, wherein the protein has at least three of the further amino acid substitutions selected from the group consisting of the sections (i), (ii), (iii), (iv), (v), (vi), (vii), (viii) and (ix) as defined in claim 4.

7. The protein according to claim 4, wherein the further amino acid substitution is selected from the group consisting of
(i) the amino acid at position 79 is S, W or I;
(ii) the amino acid at position 202 is N;
(iii) the amino acid at position 280 is A;
(iv) the amino acid at position 301 is A;
(v) the amino acid at position 3 is Q;
(vi) the amino acid at position 11 is A;
(vii) the amino acid at position 17 is P;
(viii) the amino acid at position 40 is M; and
(ix) the amino acid at position 111 is E.

8. The protein according to claim 2, wherein the protein has amino acid substitutions at position 186 and at position 79.

9. The protein according to claim 2, wherein the protein has the amino acid substitutions at position 186 and at position 79 and at position 301 and at position 40.

10. The protein according to claim 1, wherein the protein has at least one additional amino acid substitution.

11. A nucleic acid molecule encoding a protein according to claim 1.

12. The nucleic acid molecule according to claim 11 encoding a protein having the activity of a lipase selected from the group consisting of
a) nucleic acid molecules comprising the nucleic acid sequences as set forth in SEQ ID Nos. 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402; and
b) nucleic acid molecules having at least 60% identity with the nucleic acid sequences as set forth in a).

13. A recombinant nucleic acid molecule comprising a nucleic acid molecule according to claim 11.

14. The recombinant nucleic acid molecule according to claim 13, wherein the recombinant nucleic acid molecule is a vector or a plasmid.

15. A host cell comprising a protein according to claim 1.

16. A use of a protein according to claim 1 for the stereoselective acylation or carboxylation of 2,6-Dimethyl-1-indanamine.

17. The use of a protein according to claim 16, wherein the substrate is racemic 2,6-dimethyl-1-indanylamine.

18. A method for the production of an acylated or carboxylated product comprising the steps of
a) providing an acyl- or carboxy-acceptor molecule;
b) providing an acyl- or carboxy-donor molecule; and
c) contacting the acyl- or carboxy-acceptor molecule provided in step a) and the acyl- or carboxy-donor molecule provided in step b) with a protein according to the invention.

19. A host cell comprising a nucleic acid molecule according to claim 11.

20. A host cell comprising a recombinant nucleic acid molecule according to claim 13.


SEQUENCE LISTING (402 sequences)

SEQ ID NO: 1 - protein, 319 aa, unidentified
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 2 - DNA, 957 bp, unidentified
  source 1..957: mol_type = genomic DNA
atgcgtatgtctcgtcgtgccaaactgcgctgcgccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaaccctgagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcccggtagcgccgctgattggcggg

SEQ ID NO: 3 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTFSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 4 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaacctttagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 5 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDESEHLAIPFDRRALAYVLNALDPAHPRPVPCVPVAPLIGG

SEQ ID NO: 6 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatgagagcgaacatctggcaattccgtttgatcgtcgtgcactggcctatgttctgaatgccctggatccggcacatccgcgtccggttccgtgtgttccggttgcaccgctgattggcggt

SEQ ID NO: 7 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDSSEHLAIPFDRRALAYVLNALDPAHPRPVPCVPVAPLIGG

SEQ ID NO: 8 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgattctagcgaacatctggcaattccgtttgatcgtcgtgcactggcctatgttctgaatgccctggatccggcacatccgcgtccggttccgtgtgttccggttgcaccgctgattggcggt

SEQ ID NO: 9 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDKSEHLAIPFDRRALAYVLNALDPAHPRPVPCVPVAPLIGG

SEQ ID NO: 10 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgataagagcgaacatctggcaattccgtttgatcgtcgtgcactggcctatgttctgaatgccctggatccggcacatccgcgtccggttccgtgtgttccggttgcaccgctgattggcggt

SEQ ID NO: 11 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVNVAPLIGG

SEQ ID NO: 12 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttaatgttgcaccgctgattggcggt

SEQ ID NO: 13 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVFVAPLIGG

SEQ ID NO: 14 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaacctggagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 15 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 16 - DNA, 957 bp, synthetic construct; identical to SEQ ID NO: 14
  source 1..957: mol_type = other DNA

SEQ ID NO: 17 - protein, 319 aa, synthetic construct
MRLSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 18 - DNA, 957 bp, synthetic construct
  source 1..957: mol_type = other DNA
atgcgtttgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 19 - protein, 319 aa, synthetic construct
MRMSRRAKLRCALPLALVAVVAAFAPAAYAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 20 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcatatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 21 - protein, 319 aa, synthetic construct
MRMSRRAKLRCALPLAPVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 22 - DNA, 957 bp, synthetic construct
  source 1..957: mol_type = other DNA
atgcgtatgagccgtcgtgcaaaactgcgttgtgcactgccgctggcaccggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 23 - protein, 319 aa, synthetic construct
MRMPRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 24 - DNA, 957 bp, synthetic construct
  source 1..957: mol_type = other DNA
atgcgtatgccgcgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 25 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDDSEHLAIPFDRRALAYVLNALDPAHPRPVPCVPVAPLIGG

SEQ ID NO: 26 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatgatagcgaacatctggcaattccgtttgatcgtcgtgcactggcctatgttctgaatgccctggatccggcacatccgcgtccggttccgtgtgttccggttgcaccgctgattggcggt

SEQ ID NO: 27 - protein, 319 aa, synthetic construct
MRMSRRAKLRCALPLALAAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 28 - DNA, 957 bp, synthetic construct
  source 1..957: mol_type = other DNA
atgcgtatgagccgtcgtgcaaaactgcgttgtgcactgccgctggcactggcggcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 29 - protein, 319 aa, synthetic construct
MRQSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 30 - DNA, 957 bp, synthetic construct
  source 1..957: mol_type = other DNA
atgcgtcagagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 31 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVDVAPLIGG

SEQ ID NO: 32 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttgatgttgcaccgctgattggcggt

SEQ ID NO: 33 - protein, 319 aa, synthetic construct
MRMSRRAKLRCALPLALVAVVAAFAPAAWAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 34 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcatgggcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 35 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 36 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaacctatagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 37 - protein, 319 aa, synthetic construct
MRMSRRAKLRCALPLALTAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 38 - DNA, 957 bp, synthetic construct
  source 1..957: mol_type = other DNA
atgcgtatgagccgtcgtgcaaaactgcgttgtgcactgccgctggcactgacggcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 39 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTESGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 40 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccgagagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 41 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNQAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 42 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatcaggccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 43 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 44 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggcgccggcacatccgcgtccggttccgtgtgttccggttgcaccgctgattggcggt

SEQ ID NO: 45 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVCCVPVAPLIGG

SEQ ID NO: 46 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggtttgttgtgttccggttgcaccgctgattggcggt

SEQ ID NO: 47 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTDSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 48 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccgatagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 49 - protein, 319 aa, synthetic construct
MRMSRRAKLRCALPLALVAVVAAFAPAANAWLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 50 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcatggctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 51 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNNAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 52 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaataatgccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 53 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 54 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcaccgagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 55 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTQSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 56 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaacccagagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 57 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTTSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 58 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccaccagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 59 - protein, 319 aa, synthetic construct
MRCSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 60 - DNA, 957 bp, synthetic construct
  source 1..957: mol_type = other DNA
atgcgttgtagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 61 - protein, 319 aa, synthetic construct; identical to SEQ ID NO: 49

SEQ ID NO: 62 - DNA, 957 bp, synthetic construct; identical to SEQ ID NO: 50
  source 1..957: mol_type = other DNA

SEQ ID NO: 63 - protein, 319 aa, synthetic construct
MRMSRRAKLRCALPLALVAVVAAFAPAAHAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 64 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcacatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 65 - protein, 319 aa, synthetic construct
MRMSRRAELRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 66 - DNA, 957 bp, synthetic construct
  source 1..957: mol_type = other DNA
atgcgtatgagccgtcgtgcagagctgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 67 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTHSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 68 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaacccatagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 69 - protein, 319 aa, synthetic construct
MRMSRRAKLRCALPLALVAVVAAFAPAANAQLAVDYSLPKALAAWAANPNGSPPGANDWSCRPSSLHPYPVILVHGTFINQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 70 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttattaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 71 - protein, 319 aa, synthetic construct
MRMSRRAKLRCALPLALVAVVAAFAPAANAQLAVDYSLPKALAAWAANPNGSPPGANDWSCRPSSLHPYPVILVHGTFWNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 72 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcaccttttggaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 73 - protein, 319 aa, synthetic construct
MRMSRRAKLRCALPLATVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 74 - DNA, 957 bp, synthetic construct
  source 1..957: mol_type = other DNA
atgcgtatgagccgtcgtgcaaaactgcgttgtgcactgccgctggcaacggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 75 - protein, 319 aa, synthetic construct
MRMSRRAKLRCALPLALCAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 76 - DNA, 957 bp, synthetic construct
  source 1..957: mol_type = other DNA
atgcgtatgagccgtcgtgcaaaactgcgttgtgcactgccgctggcactgtgtgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 77 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTPSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 78 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaacccctagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 79 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVQVAPLIGG

SEQ ID NO: 80 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttcaggttgcaccgctgattggcggt

SEQ ID NO: 81 - protein, 319 aa, synthetic construct
MRMSRRAKLRCALPLALVAVVAAFAPAANAQLAVDYSLPMALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 82 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgatggcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 83 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVKVAPLIGG

SEQ ID NO: 84 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttaaggttgcaccgctgattggcggt

SEQ ID NO: 85 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTCSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 86 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaacctgcagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 87 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVPCVPVAPLIGG

SEQ ID NO: 88 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatgctagcgaacatctggcaattccgtttgatcgtcgtgcactggcctatgttctgaatgccctggatccggcacatccgcgtccggttccgtgtgttccggttgcaccgctgattggcggt

SEQ ID NO: 89 - protein, 319 aa, synthetic construct
MRMLRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 90 - DNA, 957 bp, synthetic construct
  source 1..957: mol_type = other DNA
atgcgtatgctgcgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 91 - protein, 319 aa, synthetic construct
MRMSRRAKLRCALPLALSAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 92 - DNA, 957 bp, synthetic construct
  source 1..957: mol_type = other DNA
atgcgtatgagccgtcgtgcaaaactgcgttgtgcactgccgctggcactgtcggcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 93 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTKSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 94 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccaagagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 95 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTSSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 96 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaacctcgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 97 - protein, 319 aa, synthetic construct
MRMSRRAKLRCALPLALVAVVAAFAPAANAQLAVDYSLPKALAAWAANPNGSPPGANDWSCRPSSLHPYPVILVHGTFTNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 98 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttactaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 99 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTNSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 100 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccaatagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 101 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTISGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 102 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccattagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 103 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTVSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 104 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccgtgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 105 - protein, 319 aa, synthetic construct
MRMSRRAKLRCALPLALVAVVAAFAPAANAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 106 - DNA, 957 bp, synthetic construct
  source 1..957: mol_type = other DNA
atgcgtatgtctcgtcgtgccaaactgcgctgcgccttaccccttgcgcttgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcccggtagcgccgctgattggcggg

SEQ ID NO: 107 - protein, 319 aa, synthetic construct
MRMSRRAKLRAALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVNVAPLIGG

SEQ ID NO: 108 - DNA, 957 bp, synthetic construct
  source 1..957: mol_type = other DNA
atgcgtatgtctcgtcgtgccaaactgcgcgcagccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcaacgtagcgccgctgattggcggg

SEQ ID NO: 109 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVPCVNVAPLIGG

SEQ ID NO: 110 - DNA, 957 bp, synthetic construct
  source 1..957: mol_type = other DNA
atgcgtatgtctcgtcgtgccaaactgcgctgcgccttaccccttgcgcttgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgccttgtgtcaacgtagcgccgctgattggcggg

SEQ ID NO: 111 - protein, 319 aa, synthetic construct
MRMSRRAKLRCALPLAPVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVNVAPLIGG

SEQ ID NO: 112 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcaacgtagcgccgctgattggcggg

SEQ ID NO: 113 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVPCVNVAPLIGG

SEQ ID NO: 114 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgccttgtgtcaacgtagcgccgctgattggcggg

SEQ ID NO: 115 - protein, 319 aa, synthetic construct
MRMSRRAKLRCALPLALVAVVAAFAPAAHAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVPCVPVAPLIGG

SEQ ID NO: 116 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcacatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgccttgtgtcccggtagcgccgctgattggcggg

SEQ ID NO: 117 - protein, 319 aa, synthetic construct
MRMSRRAKLRCALPLAPVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVCCVPVAPLIGG

SEQ ID NO: 118 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 119 - protein, 319 aa, synthetic construct
MRMPRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNNAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 120 - DNA, 957 bp, synthetic construct
  source 1..957: mol_type = other DNA
atgcgtatgcctcgtcgtgccaaactgcgctgcgccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcccggtagcgccgctgattggcggg

SEQ ID NO: 121 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNNAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVNVAPLIGG

SEQ ID NO: 122 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcaacgtagcgccgctgattggcggg

SEQ ID NO: 123 - protein, 319 aa, synthetic construct
MRMSRRAKLRCALPLALVAVVAAFAPAANAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVCCVNVAPLIGG

SEQ ID NO: 124 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgtgttgtgtcaacgtagcgccgctgattggcggg

SEQ ID NO: 125 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDAARLLGPIGNNAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVNVAPLIGG

SEQ ID NO: 126 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgacgctgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcaacgtagcgccgctgattggcggg

SEQ ID NO: 127 - protein, 319 aa, synthetic construct
MRMSRRAELRAALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVNVAPLIGG

SEQ ID NO: 128 - DNA, 957 bp, synthetic construct
  source 1..957: mol_type = other DNA
atgcgtatgtcccgtcgtgccgaactgcgcgcagccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcaacgtagcgccgctgattggcggg

SEQ ID NO: 129 - protein, 319 aa, synthetic construct
MRMSRRAKLRCALPLALVAVVAAFAPAANAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVCCVNVAPLIGG

SEQ ID NO: 130 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgtgttgtgtcaacgtagcgccgctgattggcggg

SEQ ID NO: 131 - protein, 319 aa, synthetic construct
MRMSRRAKLRCALPLALVAVVAAFAPAANAQLAVDYSLPKALAAWAANPNGSPPGANDWSCRPSSLHPYPVILVHGTFSNQRFTWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVCCVPVAPLIGG

SEQ ID NO: 132 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcacttggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 133 - protein, 319 aa, synthetic construct
MRMSRRAKLRCALPLALVAVVAAFAPAANAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDAARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVNVAPLIGG

SEQ ID NO: 134 - DNA, 957 bp, synthetic construct
  source 1..957: mol_type = other DNA
atgcgtcagtcccgtcgtgccaaactgcgctgcgccttaccccttgcgcttgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 135 - protein, 319 aa, synthetic construct
MRMSRRAKLRCALPLALVAVVAAFAPAAHAQLAVDYSLPKALAAWAANPNGSPPGANDWSCRPSSLHPYPVILVHGTFENQRFTWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNNAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 136 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcacatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcacttggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcccggtagcgccgctgattggcggg

SEQ ID NO: 137 - protein, 319 aa, synthetic construct
MRMSRRAKLRCALPLALCAVVAAFAPAANAQLAVDYSLPMALAAWAANPNGSPPGANDWSCRPSSLHPYPVILVHGTFENQRFTWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVNVAPLIGG

SEQ ID NO: 138 - DNA, 957 bp, synthetic construct
  source 1..957: mol_type = other DNA
atgcgtatgtctcgtcgtgccaaactgcgctgcgccttaccccttgcgctttgcgctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcgagaaccagcgcttcacttggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcaacgtagcgccgctgattggcggg

SEQ ID NO: 139 - protein, 319 aa, synthetic construct
MRMPRRAKLRCALPLALVAVVAAFAPAAHAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNNAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVNVAPLIGG

SEQ ID NO: 140 - DNA, 957 bp, synthetic construct
  source 1..957: mol_type = other DNA
atgcgtatgcctcgtcgtgccaaactgcgctgcgccttaccccttgcgcttgtagctgtggttgctgcctttgcacccgcggcacatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcaacgtagcgccgctgattggcggg

SEQ ID NO: 141 - protein, 319 aa, synthetic construct
MRMSRRAKLRCALPLALAAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDAARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVNVAPLIGG

SEQ ID NO: 142 - DNA, 957 bp, synthetic construct
  source 1..957: mol_type = other DNA
atgcgtatgtctcgtcgtgccaaactgcgctgcgccttaccccttgcgcttgccgctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgacgctgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcaacgtagcgccgctgattggcggg

SEQ ID NO: 143 - protein, 319 aa, synthetic construct
MRMSRRAKLRCALPLALVAVVAAFAPAAHAQLAVDYSLPKALAAWAANPNGSPPGANDWSCRPSSLHPYPVILVHGTFENQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVCCVNVAPLIGG

SEQ ID NO: 144 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcacatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgtgttgtgtcaacgtagcgccgctgattggcggg

SEQ ID NO: 145 - protein, 319 aa, synthetic construct
MRQPRRAELRCALPLALVAVVAAFAPAANAQLAVDYSLPKALAAWAANPNGSPPGANDWSCRPSSLHPYPVILVHGTFENQRFTWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVNVAPLIGG

SEQ ID NO: 146 - DNA, 957 bp, synthetic construct
  source 1..957: mol_type = other DNA
atgcgtcagccccgtcgtgccgaactgcgctgcgccttaccccttgcgcttgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcacttggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcaacgtagcgccgctgattggcggg

SEQ ID NO: 147 - protein, 319 aa, synthetic construct
MRMSRRAKLRCALPLALVAVVAAFAPAANAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 148 - DNA, 957 bp, synthetic construct
  source 1..957: mol_type = other DNA
atgcgtatgtcccgtcgtgccaaactgcgctgcgccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 149 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNNAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 150 - DNA, 957 bp, synthetic construct
  source 1..957: mol_type = other DNA
atgcgtatgtctcgtcgtgccaaactgcgctgcgccttaccccttgcgcttgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 151 - protein, 319 aa, synthetic construct
MRMSRRAKLRCALPLAPVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNNAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 152 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaataacgccgttcagcttgtcaccagcttctgtccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcccggtagcgccgctgattggcggg

SEQ ID NO: 153 - protein, 319 aa, synthetic construct
MRQSRRAKLRCALPLALVAVVAAFAPAANAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVNVAPLIGG

SEQ ID NO: 154 - DNA, 957 bp, synthetic construct
  source 1..957: mol_type = other DNA
atgcgtcagtctcgtcgtgccaaactgcgctgcgccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcaacgtagcgccgctgattggcggg

SEQ ID NO: 155 - protein, 319 aa, synthetic construct
MRMSRRAKLRCALPLALVAVVAAFAPAANAQLAVDYSLPMALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVNVAPLIGG

SEQ ID NO: 156 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcaacgtagcgccgctgattggcggg

SEQ ID NO: 157 - protein, 319 aa, synthetic construct
MRMSRRAELRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALAPAHPRPVPCVPVAPLIGG

SEQ ID NO: 158 - DNA, 957 bp, synthetic construct
  source 1..957: mol_type = other DNA
atgcgtatgtctcgtcgtgccgaactgcgctgcgccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 159 - protein, 319 aa, synthetic construct
MRMSRRAKLRCALPLAPVAVVAAFAPAANAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVCCVPVAPLIGG

SEQ ID NO: 160 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 161 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDAARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALAPAHPRPVPCVPVAPLIGG

SEQ ID NO: 162 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgacgctgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 163 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVPCVNVAPLIGG

SEQ ID NO: 164 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgccttgtgtcaacgtagcgccgctgattggcggg

SEQ ID NO: 165 - protein, 319 aa, synthetic construct
MRMSRRAKLRCALPLALVAVVAAFAPAANAQLAVDYSLPMALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVCCVPVAPLIGG

SEQ ID NO: 166 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 167 - protein, 319 aa, synthetic construct
MRMSRRAKLRAALPLALVAVVAAFAPAAWAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 168 - DNA, 957 bp, synthetic construct
  source 1..957: mol_type = other DNA
atgcgtatgtctcgtcgtgccaaactgcgcgcagccttaccccttgcgctggtagctgtggttgctgcctttgcacccgcggcatgggcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcccggtagcgccgctgattggcggg

SEQ ID NO: 169 - protein, 319 aa, synthetic construct
MRMSRRAKLRCALPLALVAVVAAFAPAAWAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNNAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 170 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcatgggcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 171 - protein, 319 aa, synthetic construct
MRMSRRAKLRCALPLAPVAVVAAFAPAAHAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 172 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcacatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcccggtagcgccgctgattggcggg

SEQ ID NO: 173 - protein, 319 aa, synthetic construct
MRMSRRAELRAALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 174 - DNA, 957 bp, synthetic construct
  source 1..957: mol_type = other DNA
atgcgtatgtctcgtcgtgccgaactgcgcgcagccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 175 - protein, 319 aa, synthetic construct
MRMPRRAKLRCALPLALVAVVAAFAPAANAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNNAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVCCVPVAPLIGG

SEQ ID NO: 176 - DNA, 957 bp, synthetic construct
  source 1..957: mol_type = other DNA
atgcgtatgcctcgtcgtgccaaactgcgctgcgccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 177 - protein, 319 aa, synthetic construct
MRMSRRAKLRCALPLALVAVVAAFAPAANAWLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNNAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVCCVPVAPLIGG

SEQ ID NO: 178 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcatggctggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 179 - protein, 319 aa, synthetic construct
MRMSRRAKLRCALPLALVAVVAAFAPAANAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVCCVNVAPLIGG

SEQ ID NO: 180 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgtgttgtgtcaacgtagcgccgctgattggcggg

SEQ ID NO: 181 - protein, 319 aa, synthetic construct
MRMSRRAKLRCALPLALVAVVAAFAPAANAWLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDAARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 182 - DNA, 957 bp, synthetic construct
  source 1..957: mol_type = other DNA
atgcgtatgtcccgtcgtgccaaactgcgctgcgccttaccccttgcgctggtagctgtggttgctgcctttgcacccgcggcaaatgcatggctggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgacgctgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 183 - protein, 319 aa, synthetic construct
MRMSRRAKLRCALPLALVAVVAAFAPAANAWLAVDYSLPKALAAWAANPNGSPPGANDWSCRPSSLHPYPVILVHGTFSNQRFTWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNNAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 184 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcatggctggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcacttggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcccggtagcgccgctgattggcggg

SEQ ID NO: 185 - protein, 319 aa, synthetic construct
MRMSRRAKLRCALPLALVAVVAAFAPAANAQLAVDYSLPKALAAWAANPNGSPPGANDWSCRPSSLHPYPVILVHGTFSNQRFTWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDAARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVPCVPVAPLIGG

SEQ ID NO: 186 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcacttggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgacgctgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgccttgtgtcccggtagcgccgctgattggcggg

SEQ ID NO: 187 - protein, 319 aa, synthetic construct
MRMSRRAKLRCALPLALVAVVAAFAPAANAQLAVDYSLPKALAAWAANPNGSPPGANDWSCRPSSLHPYPVILVHGTFSNQRFTWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDAARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVPCVPVAPLIGG

SEQ ID NO: 188 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcacttggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgacgctgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgccttgtgtcccggtagcgccgctgattggcggg

SEQ ID NO: 189 - protein, 319 aa, synthetic construct
MRMPRRAKLRCALPLAPVAVVAAFAPAAWAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 190 - DNA, 957 bp, synthetic construct
  source 1..957: mol_type = other DNA
atgcgtatgcctcgtcgtgccaaactgcgctgcgccttaccccttgcgcctgtagctgtggttgctgcctttgcacccgcggcatgggcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 191 - protein, 319 aa, synthetic construct
MRMSRRAKLRCALPLALVAVVAAFAPAAHAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDAARLLGPIGNNAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 192 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcacatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgacgctgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcccggtagcgccgctgattggcggg

SEQ ID NO: 193 - protein, 319 aa, synthetic construct
MRMSRRAKLRCALPLALVAVVAAFAPAANAQLAVDYSLPKALAAWAANPNGSPPGANDWSCRPSSLHPYPVILVHGTFSNQRFNHNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDAARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 194 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaatcataatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgacgctgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 195 - protein, 319 aa, synthetic construct
MRMSRRAKLRCALPLAPVAVVAAFAPAAHAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVCCVPVAPLIGG

SEQ ID NO: 196 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcacatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 197 - protein, 319 aa, synthetic construct
MRMSRRAKLRCALPLALVAVVAAFAPAAHAWLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALAPAHPRPVPCVPVAPLIGG

SEQ ID NO: 198 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcacatgcatggctggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 199 - protein, 319 aa, synthetic construct
MRMPRRAKLRAALPLALVAVVAAFAPAANAQLAVDYSLPKALAAWAANPNGSPPGANDWSCRPSSLHPYPVILVHGTFENQRFNHNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNNAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 200 - DNA, 957 bp, synthetic construct
  source 1..957: mol_type = other DNA
atgcgtatgcctcgtcgtgccaaactgcgcgcagccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaatcataatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcccggtagcgccgctgattggcggg

SEQ ID NO: 201 - protein, 319 aa, synthetic construct
MRMPRRAKLRCALPLALVAVVAAFAPAANAQLAVDYSLPKALAAWAANPNGSPPGANDWSCRPSSLHPYPVILVHGTFSNQRFNHNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVPCVPVAPLIGG

SEQ ID NO: 202 - DNA, 957 bp, synthetic construct
  source 1..957: mol_type = other DNA
atgcgtatgcctcgtcgtgccaaactgcgctgcgccttaccccttgcgcttgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaatcataatgccctggcacctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgccttgtgtcccggtagcgccgctgattggcggg

SEQ ID NO: 203 - protein, 319 aa, synthetic construct
MRMSRRAELRCALPLALVAVVAAFAPAANAQLAVDYSLPMALAAWAANPNGSPPGANDWSCRPSSLHPYPVILVHGTFSNQRFTWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVNVAPLIGG

SEQ ID NO: 204 - DNA, 957 bp, synthetic construct
  source 1..957: mol_type = other DNA
atgcgtatgtctcgtcgtgccgaactgcgctgcgccttaccccttgcgctggtagctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcagcaaccagcgcttcacttggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcaacgtagcgccgctgattggcggg

SEQ ID NO: 205 - protein, 319 aa, synthetic construct
MRMSRRAKLRCALPLALVAVVAAFAPAANAWLAVDYSLPKALAAWAANPNGSPPGANDWSCRPSSLHPYPVILVHGTFENQRFTWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVPCVNVAPLIGG

SEQ ID NO: 206 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcatggctggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcacttggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgccttgtgtcaacgtagcgccgctgattggcggg

SEQ ID NO: 207 - protein, 319 aa, synthetic construct
MRMSRRAKLRCALPLALVAVVAAFAPAANAWLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDAARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALAPAHPRPVPCVNVAPLIGG

SEQ ID NO: 208 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcatggctggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgacgctgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagcgccggcccatccacgtccggtgccttgtgtcaacgtagcgccgctgattggcggg

SEQ ID NO: 209 - protein, 319 aa, synthetic construct
MRMPRRAELRCALPLALVAVVAAFAPAAWAQLAVDYSLPKALAAWAANPNGSPPGANDWSCRPSSLHPYPVILVHGTFSNQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 210 - DNA, 957 bp, synthetic construct
  source 1..957: mol_type = other DNA
atgcgtatgccccgtcgtgccgaactgcgctgcgccttaccccttgcgctggtagctgtggttgctgcctttgcacccgcggcatgggcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcccggtagcgccgctgattggcggg

SEQ ID NO: 211 - protein, 319 aa, synthetic construct
MRMSRRAKLRCALPLAPAAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVCCVNVAPLIGG

SEQ ID NO: 212 - DNA, 957 bp, synthetic construct
  source 1..957: mol_type = other DNA
atgcgtatgtctcgtcgtgccaaactgcgctgcgccttaccccttgcgcctgccgctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgtgttgtgtcaacgtagcgccgctgattggcggg

SEQ ID NO: 213 - protein, 319 aa, synthetic construct
MRMSRRAKLRCALPLALVAVVAAFAPAANAQLAVDYSLPKALAAWAANPNGSPPGANDWSCRPSSLHPYPVILVHGTFSNQRFTWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVCCVNVAPLIGG

SEQ ID NO: 214 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcacttggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgtgttgtgtcaacgtagcgccgctgattggcggg

SEQ ID NO: 215 - protein, 319 aa, synthetic construct
MRMSRRAKLRCALPLALVAVVAAFAPAAHAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDAARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALAPAHPRPVPCVNVAPLIGG

SEQ ID NO: 216 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcacatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgacgctgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagcgccggcccatccacgtccggtgccttgtgtcaacgtagcgccgctgattggcggg

SEQ ID NO: 217 - protein, 319 aa, synthetic construct
MRMSRRAELRAALPLALVAVVAAFAPAAHAWLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 218 - DNA, 957 bp, synthetic construct
  source 1..957: mol_type = other DNA
atgcgtatgtctcgtcgtgccgaactgcgcgcagccttaccccttgcgctggtagctgtggttgctgcctttgcacccgcggcacatgcatggctggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 219 - protein, 319 aa, synthetic construct
MRMPRRAKLRCALPLALVAVVAAFAPAAHAWLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALAPAHPRPVPCVPVAPLIGG

SEQ ID NO: 220 - DNA, 957 bp, synthetic construct
  source 1..957: mol_type = other DNA
atgcgtatgcctcgtcgtgccaaactgcgctgcgccttaccccttgcgctggtagctgtggttgctgcctttgcacccgcggcacatgcatggctggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaaccctgagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 221 - protein, 319 aa, synthetic construct
MRMSRRAELRCALPLALVAVVAAFAPAAHAWLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 222 - DNA, 957 bp, synthetic construct
  source 1..957: mol_type = other DNA
atgcgtatgtctcgtcgtgccgaactgcgctgcgccttaccccttgcgctggtagctgtggttgctgcctttgcacccgcggcacatgcatggctggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 223 - protein, 319 aa, synthetic construct
MRMSRRAELRCALPLAPAAVVAAFAPAANAQLAVDYSLPKALAAWAANPNGSPPGANDWSCRPSSLHPYPVILVHGTFENQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVCCVPVAPLIGG

SEQ ID NO: 224 - DNA, 957 bp, synthetic construct
  source 1..957: mol_type = other DNA
atgcgtatgtctcgtcgtgccgaactgcgctgcgccttaccccttgcgcctgccgctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 225 - protein, 319 aa, synthetic construct
MRMSRRAKLRAALPLALVAVVAAFAPAAWAQLAVDYSLPKALAAWAANPNGSPPGANDWSCRPSSLHPYPVILVHGTFSNQRFTHNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVCCVPVAPLIGG

SEQ ID NO: 226 - DNA, 957 bp, synthetic construct
  source 1..957: mol_type = other DNA
atgcgtatgtctcgtcgtgccaaactgcgcgcagccttaccccttgcgctggtagctgtggttgctgcctttgcacccgcggcatgggcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcactcataatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 227 - protein, 319 aa, synthetic construct
MRMSRRAKLRCALPLALVAVVAAFAPAAHAQLAVDYSLPKALAAWAANPNGSPPGANDWSCRPSSLHPYPVILVHGTFENQRFTWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVCCVNVAPLIGG

SEQ ID NO: 228 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcacatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcacttggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgtgttgtgtcaacgtagcgccgctgattggcggg

SEQ ID NO: 229 - protein, 319 aa, synthetic construct
MRMSRRAKLRCALPLALVAVVAAFAPAAHAWLAVDYSLPKALAAWAANPNGSPPGANDWSCRPSSLHPYPVILVHGTFSNQRFTHNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDAARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVCCVPVAPLIGG

SEQ ID NO: 230 - DNA, 957 bp, synthetic construct
  source 1..957: mol_type = other DNA
atgcgtatgtcccgtcgtgccaaactgcgctgcgccttaccccttgcgcttgtagctgtggttgctgcctttgcacccgcggcacatgcatggctggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcactcataatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgacgctgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 231 - protein, 319 aa, synthetic construct
MRMSRRAKLRCALPLAPVAVVAAFAPAANAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNNAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 232 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcccggtagcgccgctgattggcggg

SEQ ID NO: 233 - protein, 319 aa, synthetic construct
MRMSRRAKLRCALPLALVAVVAAFAPAANAQLAVDYSLPMALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 234 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 235 - protein, 319 aa, synthetic construct
MRMSRRAKLRCALPLALVAVVAAFAPAANAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVPCVPVAPLIGG

SEQ ID NO: 236 - DNA, 957 bp, synthetic construct
  source 1..957: mol_type = other DNA
atgcgtatgtctcgtcgtgccaaactgcgctgcgccttaccccttgcgcttgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgccttgtgtcccggtagcgccgctgattggcggg

SEQ ID NO: 237 - protein, 319 aa, synthetic construct
MRMSRRAKLRCALPLAPVAVVAAFAPAANAQLAVDYSLPKALAAWAANPNGSPPGANDWSCRPSSLHPYPVILVHGTFENQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 238 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 239 - protein, 319 aa, synthetic construct
MRMSRRAKLRCALPLAPVAVVAAFAPAANAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNNAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 240 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 241 - protein, 319 aa, synthetic construct
MRQSRRAKLRCALPLALVAVVAAFAPAANAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 242 - DNA, 957 bp, synthetic construct; identical to SEQ ID NO: 134
  source 1..957: mol_type = other DNA

SEQ ID NO: 243 - protein, 319 aa, synthetic construct
MRMSRRAKLRCALPLAPVAVVAAFAPAANAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALAPAHPRPVPCVPVAPLIGG

SEQ ID NO: 244 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 245 - protein, 319 aa, synthetic construct
MRMSRRAKLRCALPLAPVAVVAAFAPAANAQLAVDYSLPKALAAWAANPNGSPPGANDWSCRPSSLHPYPVILVHGTFSNQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVCCVPVAPLIGG

SEQ ID NO: 246 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 247 - protein, 319 aa, synthetic construct
MRMSRRAKLRCALPLAPCAVVAAFAPAANAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVPCVPVAPLIGG

SEQ ID NO: 248 - DNA, 957 bp, synthetic construct
  source 1..957: mol_type = other DNA
atgcgtatgtcccgtcgtgccaaactgcgctgcgccttaccccttgcgccttgcgctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgccttgtgtcccggtagcgccgctgattggcggg

SEQ ID NO: 249 - protein, 319 aa, synthetic construct
MRMSRRAKLRCALPLALVAVVAAFAPAAHAQLAVDYSLPMALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 250 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcacatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 251 - protein, 319 aa, synthetic construct
MRMSRRAKLRCALPLALVAVVAAFAPAAHAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVPCVPVAPLIGG

SEQ ID NO: 252 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcacatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgccttgtgtcccggtagcgccgctgattggcggg

SEQ ID NO: 253 - protein, 319 aa, synthetic construct
MRMSRRAKLRCALPLALVAVVAAFAPAAHAQLAVDYSLPKALAAWAANPNGSPPGANDWSCRPSSLHPYPVILVHGTFSNQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVPCVPVAPLIGG

SEQ ID NO: 254 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcacatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgccttgtgtcccggtagcgccgctgattggcggg

SEQ ID NO: 255 - protein, 319 aa, synthetic construct
MRMSRRAKLRCALPLALVAVVAAFAPAAHAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNNAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 256 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcacatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 257 - protein, 319 aa, synthetic construct
MRMSRRAKLRAALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALAPAHPRPVCCVPVAPLIGG

SEQ ID NO: 258 - DNA, 957 bp, synthetic construct
  source 1..957: mol_type = other DNA
atgcgtatgtctcgtcgtgccaaactgcgcgcagccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagcgccggcccatccacgtccggtgtgttgtgtcccggtagcgccgctgattggcggg

SEQ ID NO: 259 - protein, 319 aa, synthetic construct
MRMSRRAELRCALPLALVAVVAAFAPAANAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALAPAHPRPVPCVPVAPLIGG

SEQ ID NO: 260 - DNA, 957 bp, synthetic construct
  source 1..957: mol_type = other DNA
atgcgtatgtctcgtcgtgccgaactgcgctgcgccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 261 - protein, 319 aa, synthetic construct
MRMSRRAKLRCALPLAPVAVVAAFAPAAWAQLAVDYSLPKALAAWAANPNGSPPGANDWSCRPSSLHPYPVILVHGTFENQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTWSGLTDFARLLGPIGNNAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 262 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcatgggcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcccggtagcgccgctgattggcggg

SEQ ID NO: 263 - protein, 319 aa, synthetic construct
MRMSRRAKLRCALPLAPVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALAPAHPRPVCCVPVAPLIGG

SEQ ID NO: 264 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagcgccggcccatccacgtccggtgtgttgtgtcccggtagcgccgctgattggcggg

SEQ ID NO: 265 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTWSGLTDFARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVPCVNVAPLIGG

SEQ ID NO: 266 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgccttgtgtcaacgtagcgccgctgattggcggg

SEQ ID NO: 267 - protein, 319 aa, synthetic construct
MRMSRRAKLRCALPLALVAVVAAFAPAAHAQLAVDYSLPKALAAWAANPNGSPPGANDWSCRPSSLHPYPVILVHGTFSNQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 268 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcacatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 269 - protein, 319 aa, synthetic construct
MRMSRRAKLRCALPLAPVAVVAAFAPAAWAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNNAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVNVAPLIGG

SEQ ID NO: 270 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcatgggcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcaacgtagcgccgctgattggcggg

SEQ ID NO: 271 - protein, 319 aa, synthetic construct
MRMSRRAKLRCALPLAPVAVVAAFAPAANAQLAVDYSLPKALAAWAANPNGSPPGANDWSCRPSSLHPYPVILVHGTFSNQRFTWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVCCVPVAPLIGG

SEQ ID NO: 272 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcacttggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 273 - protein, 319 aa, synthetic construct
MRMSRRAKLRCALPLAPVAVVAAFAPAAHAQLAVDYSLPKALAAWAANPNGSPPGANDWSCRPSSLHPYPVILVHGTFSNQRFNHNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVPCVPVAPLIGG

SEQ ID NO: 274 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcacatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaatcataatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgccttgtgtcccggtagcgccgctgattggcggg

SEQ ID NO: 275 - protein, 319 aa, synthetic construct
MRMSRRAKLRCALPLAPVAVVAAFAPAANAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDAARLLGPIGNNAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVCCVPVAPLIGG

SEQ ID NO: 276 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattctccaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgacgctgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 277 - protein, 319 aa, synthetic construct
MRMSRRAKLRCALPLALAAVVAAFAPAANAQLAVDYSLPMALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDAARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 278 - DNA, 957 bp, synthetic construct
  source 1..957: mol_type = other DNA
atgcgtatgtcccgtcgtgccaaactgcgctgcgccttaccccttgcgcttgccgctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgacgctgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 279 - protein, 319 aa, synthetic construct
MRMSRRAKLRCALPLAPVAVVAAFAPAAHAWLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 280 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcacatgcatggctggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 281 - protein, 319 aa, synthetic construct
MRMSRRAELRCALPLALVAVVAAFAPAANAQLAVDYSLPMALAAWAANPNGSPPGANDWSCRPSSLHPYPVILVHGTFENQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVCCVPVAPLIGG

SEQ ID NO: 282 - DNA, 957 bp, synthetic construct
  source 1..957: mol_type = other DNA
atgcgtatgtctcgtcgtgccgaactgcgctgcgccttaccccttgcgctggtagctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 283 - protein, 319 aa, synthetic construct
MRQSRRAELRCALPLAPVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVNVAPLIGG

SEQ ID NO: 284 - DNA, 957 bp, synthetic construct
  source 1..957: mol_type = other DNA
atgcgtcagtcccgtcgtgccgaactgcgctgcgccttaccccttgcgcctgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcaacgtagcgccgctgattggcggg

SEQ ID NO: 285 - protein, 319 aa, synthetic construct
MRMSRRAKLRCALPLAPVAVVAAFAPAAWAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVCCVPVAPLIGG

SEQ ID NO: 286 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcatgggcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 287 - protein, 319 aa, synthetic construct
MRMSRRAELRAALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVCCVPVAPLIGG

SEQ ID NO: 288 - DNA, 957 bp, synthetic construct
  source 1..957: mol_type = other DNA
atgcgtatgtctcgtcgtgccgaactgcgcgcagccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 289 - protein, 319 aa, synthetic construct
MRMPRRAKLRCALPLALVAVVAAFAPAAWAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVCCVPVAPLIGG

SEQ ID NO: 290 - DNA, 962 bp, synthetic construct
  source 1..962: mol_type = other DNA
atcatatgcgtatgccccgtcgtgccaaactgcgctgcgccttaccccttgcgctggtagctgtggttgctgcctttgcacccgcggcatgggcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 291 - protein, 319 aa, synthetic construct
MRMSRRAKLRCALPLALVAVVAAFAPAAWAQLAVDYSLPKALAAWAANPNGSPPGANDWSCRPSSLHPYPVILVHGTFSNQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVCCVNVAPLIGG

SEQ ID NO: 292 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcatgggcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgtgttgtgtcaacgtagcgccgctgattggcggg

SEQ ID NO: 293 - protein, 319 aa, synthetic construct
MRMSRRAELRAALPLALVAVVAAFAPAANAQLAVDYSLPMALAAWAANPNGSPPGANDWSCRPSSLHPYPVILVHGTFSNQRFNHNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVCCVPVAPLIGG

SEQ ID NO: 294 - DNA, 957 bp, synthetic construct
  source 1..957: mol_type = other DNA
atgcgtatgtcccgtcgtgccgaactgcgcgcagccttaccccttgcgctggtagctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcagcaaccagcgcttcaatcataatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 295 - protein, 319 aa, synthetic construct
MRQPRRAKLRCALPLALVAVVAAFAPAAHAQLAVDYSLPMALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNNAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVCCVPVAPLIGG

SEQ ID NO: 296 - DNA, 957 bp, synthetic construct
  source 1..957: mol_type = other DNA
atgcgtcagccccgtcgtgccaaactgcgctgcgccttaccccttgcgcttgtagctgtggttgctgcctttgcacccgcggcacatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 297 - protein, 319 aa, synthetic construct
MRMSRRAKLRCALPLALAAVVAAFAPAANAQLAVDYSLPMALAAWAANPNGSPPGANDWSCRPSSLHPYPVILVHGTFENQRFTWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVCCVNVAPLIGG

SEQ ID NO: 298 - DNA, 957 bp, synthetic construct
  source 1..957: mol_type = other DNA
atgcgtatgtctcgtcgtgccaaactgcgctgcgccttaccccttgcgcttgccgctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcgagaaccagcgcttcacttggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgtgttgtgtcaacgtagcgccgctgattggcggg

SEQ ID NO: 299 - protein, 319 aa, synthetic construct
MRMPRRAKLRAALPLALAAVVAAFAPAANAQLAVDYSLPMALAAWAANPNGSPPGANDWSCRPSSLHPYPVILVHGTFENQRFTWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVNVAPLIGG

SEQ ID NO: 300 - DNA, 957 bp, synthetic construct
  source 1..957: mol_type = other DNA
atgcgtatgccccgtcgtgccaaactgcgcgcagccttaccccttgcgcttgccgctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcgagaaccagcgcttcacttggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcaacgtagcgccgctgattggcggg

SEQ ID NO: 301 - protein, 319 aa, synthetic construct
MRMPRRAELRAALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDAARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVCCVPVAPLIGG

SEQ ID NO: 302 - DNA, 957 bp, synthetic construct
  source 1..957: mol_type = other DNA
atgcgtatgccccgtcgtgccgaactgcgcgcagccttaccccttgcgcttgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgacgctgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 303 - protein, 319 aa, synthetic construct
MRMSRRAKLRCALPLALVAVVAAFAPAANAQLAVDYSLPKALAAWAANPNGSPPGANDWSCRPSSLHPYPVILVHGTFSNQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALAPAHPRPVPCVPVAPLIGG

SEQ ID NO: 304 - DNA, 957 bp, synthetic construct
  source 1..957: mol_type = other DNA
atgcgtatgtctcgtcgtgccaaactgcgctgcgccttaccccttgcgcttgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 305 - protein, 319 aa, synthetic construct
MRQSRRAKLRCALPLAPVAVVAAFAPAANAQLAVDYSLPMALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 306 - DNA, 957 bp, synthetic construct
  source 1..957: mol_type = other DNA
atgcgtcagtctcgtcgtgccaaactgcgctgcgccttaccccttgcgcctgtagctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcccggtagcgccgctgattggcggg

SEQ ID NO: 307 - protein, 319 aa, synthetic construct
MRMSRRAKLRCALPLAPVAVVAAFAPAANAQLAVDYSLPMALAAWAANPNGSPPGANDWSCRPSSLHPYPVILVHGTFENQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVPCVPVAPLIGG

SEQ ID NO: 308 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgccttgtgtcccggtagcgccgctgattggcggg

SEQ ID NO: 309 - protein, 319 aa, synthetic construct
MRMSRRAKLRAALPLALVAVVAAFAPAANAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNNAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 310 - DNA, 957 bp, synthetic construct
  source 1..957: mol_type = other DNA
atgcgtatgtctcgtcgtgccaaactgcgcgcagccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 311 - protein, 319 aa, synthetic construct
MRMSRRAKLRCALPLAPVAVVAAFAPAANAQLAVDYSLPMALAAWAANPNGSPPGANDWSCRPSSLHPYPVILVHGTFSNQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 312 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcccggtagcgccgctgattggcggg

SEQ ID NO: 313 - protein, 319 aa, synthetic construct
MRMSRRAKLRAALPLAPVAVVAAFAPAANAQLAVDYSLPMALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 314 - DNA, 957 bp, synthetic construct
  source 1..957: mol_type = other DNA
atgcgtatgtctcgtcgtgccaaactgcgcgcagccttaccccttgcgcctgtagctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcccggtagcgccgctgattggcggg

SEQ ID NO: 315 - protein, 319 aa, synthetic construct
MRMSRRAKLRCALPLALVAVVAAFAPAANAQLAVDYSLPMALAAWAANPNGSPPGANDWSCRPSSLHPYPVILVHGTFENQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALAPAHPRPVPCVPVAPLIGG

SEQ ID NO: 316 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 317 - protein, 319 aa, synthetic construct
MRMSRRAKLRCALPLALVAVVAAFAPAANAQLAVDYSLPKALAAWAANPNGSPPGANDWSCRPSSLHPYPVILVHGTFSNQRFTWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALAPAHPRPVPCVPVAPLIGG

SEQ ID NO: 318 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcacttggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 319 - protein, 319 aa, synthetic construct
MRQSRRAKLRCALPLAPVAVVAAFAPAAHAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 320 - DNA, 957 bp, synthetic construct
  source 1..957: mol_type = other DNA
atgcgtcagtcccgtcgtgccaaactgcgctgcgccttaccccttgcgcctgtagctgtggttgctgcctttgcacccgcggcacatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 321 - protein, 319 aa, synthetic construct
MRMSRRAKLRCALPLAPVAVVAAFAPAANAQLAVDYSLPKALAAWAANPNGSPPGANDWSCRPSSLHPYPVILVHGTFENQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTYSGLTDFARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVPCVNVAPLIGG

SEQ ID NO: 322 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgccttgtgtcaacgtagcgccgctgattggcggg

SEQ ID NO: 323 - protein, 319 aa, synthetic construct
MRQSRRAKLRAALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVPCVNVAPLIGG

SEQ ID NO: 324 - DNA, 957 bp, synthetic construct
  source 1..957: mol_type = other DNA
atgcgtcagtcccgtcgtgccaaactgcgcgcagccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgccttgtgtcaacgtagcgccgctgattggcggg

SEQ ID NO: 325 - protein, 319 aa, synthetic construct
MRMSRRAKLRCALPLAPVAVVAAFAPAANAQLAVDYSLPKALAAWAANPNGSPPGANDWSCRPSSLHPYPVILVHGTFSNQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTYSGLTDAARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 326 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgacgctgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 327 - protein, 319 aa, synthetic construct
MRMSRRAKLRAALPLALVAVVAAFAPAANAQLAVDYSLPKALAAWAANPNGSPPGANDWSCRPSSLHPYPVILVHGTFSNQRFNHNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVPCVNVAPLIGG

SEQ ID NO: 328 - DNA, 957 bp, synthetic construct
  source 1..957: mol_type = other DNA
atgcgtatgtctcgtcgtgccaaactgcgcgcagccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaatcataatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgccttgtgtcaacgtagcgccgctgattggcggg

SEQ ID NO: 329 - protein, 319 aa, synthetic construct
MRMSRRAKLRCALPLAPVAVVAAFAPAANAQLAVDYSLPMALAAWAANPNGSPPGANDWSCRPSSLHPYPVILVHGTFSNQRFTWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNNAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVCCVPVAPLIGG

SEQ ID NO: 330 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcagcaaccagcgcttcacttggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 331 - protein, 319 aa, synthetic construct
MRMSRRAKLRAALPLALVAVVAAFAPAANAWLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALAPAHPRPVCCVPVAPLIGG

SEQ ID NO: 332 - DNA, 957 bp, synthetic construct
  source 1..957: mol_type = other DNA
atgcgtatgtctcgtcgtgccaaactgcgcgcagccttaccccttgcgctggtagctgtggttgctgcctttgcacccgcggcaaatgcatggctggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagcgccggcccatccacgtccggtgtgttgtgtcccggtagcgccgctgattggcggg

SEQ ID NO: 333 - protein, 319 aa, synthetic construct
MRMSRRAELRCALPLALVAVVAAFAPAANAQLAVDYSLPMALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNNAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVNVAPLIGG

SEQ ID NO: 334 - DNA, 957 bp, synthetic construct
  source 1..957: mol_type = other DNA
atgcgtatgtcccgtcgtgccgaactgcgctgcgccttaccccttgcgctggtagctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcaacgtagcgccgctgattggcggg

SEQ ID NO: 335 - protein, 319 aa, synthetic construct
MRQSRRAKLRAALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVCCVNVAPLIGG

SEQ ID NO: 336 - DNA, 957 bp, synthetic construct
  source 1..957: mol_type = other DNA
atgcgtcagtctcgtcgtgccaaactgcgcgcagccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgtgttgtgtcaacgtagcgccgctgattggcggg

SEQ ID NO: 337 - protein, 319 aa, synthetic construct
MRMSRRAKLRCALPLAPVAVVAAFAPAANAQLAVDYSLPMALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDAARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVCCVPVAPLIGG

SEQ ID NO: 338 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgacgctgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 339 - protein, 319 aa, synthetic construct
MRMSRRAELRCALPLAPVAVVAAFAPAAWAQLAVDYSLPMALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDAARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVPCVPVAPLIGG

SEQ ID NO: 340 - DNA, 957 bp, synthetic construct
  source 1..957: mol_type = other DNA
atgcgtatgtctcgtcgtgccgaactgcgctgcgccttaccccttgcgcctgtagctgtggttgctgcctttgcacccgcggcatgggcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgacgctgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgccttgtgtcccggtagcgccgctgattggcggg

SEQ ID NO: 341 - protein, 319 aa, synthetic construct
MRMPRRAKLRCALPLALVAVVAAFAPAAHAQLAVDYSLPKALAAWAANPNGSPPGANDWSCRPSSLHPYPVILVHGTFENQRFTWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTYSGLTDFARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALAPAHPRPVPCVPVAPLIGG

SEQ ID NO: 342 - DNA, 957 bp, synthetic construct
  source 1..957: mol_type = other DNA
atgcgtatgcctcgtcgtgccaaactgcgctgcgccttaccccttgcgctggtagctgtggttgctgcctttgcacccgcggcacatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcacttggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 343 - protein, 319 aa, synthetic construct
MRMSRRAKLRCALPLAPVAVVAAFAPAANAQLAVDYSLPMALAAWAANPNGSPPGANDWSCRPSSLHPYPVILVHGTFSNQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTLSGLTDAARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVCCVNVAPLIGG

SEQ ID NO: 344 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaaccctgagtggcctgactgacgctgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgtgttgtgtcaacgtagcgccgctgattggcggg

SEQ ID NO: 345 - protein, 319 aa, synthetic construct
MRQSRRAKLRCALPLAPCAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVCCVNVAPLIGG

SEQ ID NO: 346 - DNA, 957 bp, synthetic construct
  source 1..957: mol_type = other DNA
atgcgtcagtctcgtcgtgccaaactgcgctgcgccttaccccttgcgccttgcgctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgtgttgtgtcaacgtagcgccgctgattggcggg

SEQ ID NO: 347 - protein, 319 aa, synthetic construct
MRQPRRAELRAALPLALVAVVAAFAPAANAQLAVDYSLPKALAAWAANPNGSPPGANDWSCRPSSLHPYPVILVHGTFSNQRFNHNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDAARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 348 - DNA, 957 bp, synthetic construct
  source 1..957: mol_type = other DNA
atgcgtcagccccgtcgtgccgaactgcgcgcagccttaccccttgcgcttgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaatcataatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgacgctgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 349 - protein, 319 aa, synthetic construct
MRMSRRAELRCALPLALVAVVAAFAPAANAQLAVDYSLPKALAAWAANPNGSPPGANDWSCRPSSLHPYPVILVHGTFSNQRFNHNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTYSGLTDAARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVCCVPVAPLIGG

SEQ ID NO: 350 - DNA, 957 bp, synthetic construct
  source 1..957: mol_type = other DNA
atgcgtatgtctcgtcgtgccgaactgcgctgcgccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaatcataatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgacgctgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 351 - protein, 319 aa, synthetic construct
MRMSRRAELRCALPLALVAVVAAFAPAANAQLAVDYSLPMALAAWAANPNGSPPGANDWSCRPSSLHPYPVILVHGTFSNQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALAPAHPRPVPCVPVAPLIGG

SEQ ID NO: 352 - DNA, 957 bp, synthetic construct
  source 1..957: mol_type = other DNA
atgcgtatgtctcgtcgtgccgaactgcgctgcgccttaccccttgcgctggtagctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 353 - protein, 319 aa, synthetic construct
MRMSRRAKLRCALPLAPVAVVAAFAPAANAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDAARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALAPAHPRPVPCVPVAPLIGG

SEQ ID NO: 354 - DNA, 957 bp, synthetic construct
  source 1..957: mol_type = other DNA
atgcgtatgtcccgtcgtgccaaactgcgctgcgccttaccccttgcgcctgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgacgctgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 355 - protein, 319 aa, synthetic construct
MRMSRRAKLRCALPLALVAVVAAFAPAANAWLAVDYSLPKALAAWAANPNGSPPGANDWSCRPSSLHPYPVILVHGTFSNQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTWSGLTDFARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALAPAHPRPVPCVPVAPLIGG

SEQ ID NO: 356 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcatggctggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 357 - protein, 319 aa, synthetic construct
MRMSRRAKLRCALPLAPVAVVAAFAPAANAQLAVDYSLPMALAAWAANPNGSPPGANDWSCRPSSLHPYPVILVHGTFSNQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTWSGLTDAARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 358 - DNA, 957 bp, synthetic construct
  source 1..957: mol_type = other DNA
atgcgtatgtcccgtcgtgccaaactgcgctgcgccttaccccttgcgcctgtagctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgacgctgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 359 - protein, 319 aa, synthetic construct
MRMSRRAKLRAALPLALVAVVAAFAPAANAQLAVDYSLPMALAAWAANPNGSPPGANDWSCRPSSLHPYPVILVHGTFSNQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTWSGLTDAARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 360 - DNA, 957 bp, synthetic construct
  source 1..957: mol_type = other DNA
atgcgtatgtctcgtcgtgccaaactgcgcgcagccttaccccttgcgctggtagctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgacgctgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 361 - protein, 319 aa, synthetic construct
MRMSRRAKLRCALPLAPAAVVAAFAPAANAQLAVDYSLPMALAAWAANPNGSPPGANDWSCRPSSLHPYPVILVHGTFENQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTYSGLTDFARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVPCVPVAPLIGG

SEQ ID NO: 362 - DNA, 957 bp, synthetic construct
  source 1..957: mol_type = other DNA
atgcgtatgtctcgtcgtgccaaactgcgctgcgccttaccccttgcgcctgccgctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgccttgtgtcccggtagcgccgctgattggcggg

SEQ ID NO: 363 - protein, 319 aa, synthetic construct
MRQSRRAKLRCALPLAPVAVVAAFAPAANAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNNAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVCCVPVAPLIGG

SEQ ID NO: 364 - DNA, 957 bp, synthetic construct
  source 1..957: mol_type = other DNA
atgcgtcagtctcgtcgtgccaaactgcgctgcgccttaccccttgcgcctgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgtgttgtgtcccggtagcgccgctgattggcggg

SEQ ID NO: 365 - protein, 319 aa, synthetic construct
MRQPRRAKLRAALPLAPVAVVAAFAPAANAQLAVDYSLPKALAAWAANPNGSPPGANDWSCRPSSLHPYPVILVHGTFSNQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 366 - DNA, 957 bp, synthetic construct
  source 1..957: mol_type = other DNA
atgcgtcagccccgtcgtgccaaactgcgcgcagccttaccccttgcgcctgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcccggtagcgccgctgattggcggg

SEQ ID NO: 367 - protein, 319 aa, synthetic construct
MRMSRRAKLRCALPLALVAVVAAFAPAANAQLAVDYSLPMALAAWAANPNGSPPGANDWSCRPSSLHPYPVILVHGTFSNQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTYSGLTDFARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVCCVNVAPLIGG

SEQ ID NO: 368 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgtgttgtgtcaacgtagcgccgctgattggcggg

SEQ ID NO: 369 - protein, 319 aa, synthetic construct
MRMSRRAKLRAALPLALVAVVAAFAPAAWAWLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALAPAHPRPVPCVPVAPLIGG

SEQ ID NO: 370 - DNA, 957 bp, synthetic construct
  source 1..957: mol_type = other DNA
atgcgtatgtctcgtcgtgccaaactgcgcgcagccttaccccttgcgctggtagctgtggttgctgcctttgcacccgcggcatgggcatggctggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 371 - protein, 319 aa, synthetic construct
MRMSRRAKLRCALPLAPVAVVAAFAPAANAQLAVDYSLPMALAAWAANPNGSPPGANDWSCRPSSLHPYPVILVHGTFSNQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTWSGLTDAARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVCCVPVAPLIGG

SEQ ID NO: 372 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgacgctgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 373 - protein, 319 aa, synthetic construct
MRMSRRAKLRCALPLAPVAVVAAFAPAAHAWLAVDYSLPMALAAWAANPNGSPPGANDWSCRPSSLHPYPVILVHGTFSNQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVPCVPVAPLIGG

SEQ ID NO: 374 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcacatgcatggctggcggtggactatagcctgccgatggcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgccttgtgtcccggtagcgccgctgattggcggg

SEQ ID NO: 375 - protein, 319 aa, synthetic construct
MRMSRRAELRCALPLALVAVVAAFAPAANAQLAVDYSLPMALAAWAANPNGSPPGANDWSCRPSSLHPYPVILVHGTFSNQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTWSGLTDFARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVCCVPVAPLIGG

SEQ ID NO: 376 - DNA, 957 bp, synthetic construct
  source 1..957: mol_type = other DNA
atgcgtatgtcccgtcgtgccgaactgcgctgcgccttaccccttgcgctggtagctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 377 - protein, 319 aa, synthetic construct
MRMPRRAKLRCALPLALVAVVAAFAPAANAQLAVDYSLPMALAAWAANPNGSPPGANDWSCRPSSLHPYPVILVHGTFSNQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTYSGLTDFARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVCCVPVAPLIGG

SEQ ID NO: 378 - DNA, 957 bp, synthetic construct
  source 1..957: mol_type = other DNA
atgcgtatgcctcgtcgtgccaaactgcgctgcgccttaccccttgcgctggtagctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 379 - protein, 319 aa, synthetic construct
MRQSRRAKLRCALPLAPVAVVAAFAPAAWAWLAVDYSLPKALAAWAANPNGSPPGANDWSCRPSSLHPYPVILVHGTFENQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTYSGLTDFARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVCCVPVAPLIGG

SEQ ID NO: 380 - DNA, 957 bp, synthetic construct
  source 1..957: mol_type = other DNA
atgcgtcagtctcgtcgtgccaaactgcgctgcgccttaccccttgcgcctgtagctgtggttgctgcctttgcacccgcggcatgggcatggctggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 381 - protein, 319 aa, synthetic construct
MRMSRRAKLRCALPLAPVAVVAAFAPAANAQLAVDYSLPMALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALAPAHPRPVPCVPVAPLIGG

SEQ ID NO: 382 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 383 - protein, 319 aa, synthetic construct
MRQSRRAKLRCALPLAPVAVVAAFAPAANAQLAVDYSLPKALAAWAANPNGSPPGANDWSCRPSSLHPYPVILVHGTFSNQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTWSGLTDAARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVPCVPVAPLIGG

SEQ ID NO: 384 - DNA, 957 bp, synthetic construct
  source 1..957: mol_type = other DNA
atgcgtcagtctcgtcgtgccaaactgcgctgcgccttaccccttgcgcctgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgacgctgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgccttgtgtcccggtagcgccgctgattggcggg

SEQ ID NO: 385 - protein, 319 aa, synthetic construct
MRQSRRAKLRCALPLAPAAVVAAFAPAANAQLAVDYSLPKALAAWAANPNGSPPGANDWSCRPSSLHPYPVILVHGTFSNQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALAPAHPRPVPCVNVAPLIGG

SEQ ID NO: 386 - DNA, 957 bp, synthetic construct
  source 1..957: mol_type = other DNA
atgcgtcagtctcgtcgtgccaaactgcgctgcgccttaccccttgcgcctgccgctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagcgccggcccatccacgtccggtgccttgtgtcaacgtagcgccgctgattggcggg

SEQ ID NO: 387 - protein, 319 aa, synthetic construct
MRQSRRAKLRCALPLAPAAVVAAFAPAANAQLAVDYSLPMALAAWAANPNGSPPGANDWSCRPSSLHPYPVILVHGTFSNQRFTWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVPCVPVAPLIGG

SEQ ID NO: 388 - DNA, 957 bp, synthetic construct
  source 1..957: mol_type = other DNA
atgcgtcagtctcgtcgtgccaaactgcgctgcgccttaccccttgcgcctgccgctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcagcaaccagcgcttcacttggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgccttgtgtcccggtagcgccgctgattggcggg

SEQ ID NO: 389 - protein, 319 aa, synthetic construct
MRMSRRAKLRCALPLAPVAVVAAFAPAANAQLAVDYSLPMALAAWAANPNGSPPGANDWSCRPSSLHPYPVILVHGTFSNQRFTWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTYSGLTDFARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALAPAHPRPVPCVPVAPLIGG

SEQ ID NO: 390 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcagcaaccagcgcttcacttggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 391 - protein, 319 aa, synthetic construct
MRQPRRAKLRCALPLAPVAVVAAFAPAANAQLAVDYSLPMALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALAPAHPRPVPCVPVAPLIGG

SEQ ID NO: 392 - DNA, 957 bp, synthetic construct
  source 1..957: mol_type = other DNA
atgcgtcagcctcgtcgtgccaaactgcgctgcgccttaccccttgcgcctgtagctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 393 - protein, 319 aa, synthetic construct
MRMSRRAKLRCALPLALVAVVAAFAPAAWAWLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVNVAPLIGG

SEQ ID NO: 394 - DNA, 957 bp, synthetic construct
  source 1..957: mol_type = other DNA
atgcgtatgtcccgtcgtgccaaactgcgctgcgccttaccccttgcgctggtagctgtggttgctgcctttgcacccgcggcatgggcatggctggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcaacgtagcgccgctgattggcggg

SEQ ID NO: 395 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDAARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVCCVNVAPLIGG

SEQ ID NO: 396 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgacgctgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgtgttgtgtcaacgtagcgccgctgattggcggg

SEQ ID NO: 397 - protein, 319 aa, synthetic construct
MRMSRRAKLRCALPLALVAVVAAFAPAAWAWLAVDYSLPKALAAWAANPNGSPPGANDWSCRPSSLHPYPVILVHGTFENQRFNHNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVNVAPLIGG

SEQ ID NO: 398 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcatgggcatggctggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaatcataatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcaacgtagcgccgctgattggcggg

SEQ ID NO: 399 - protein, 319 aa, synthetic construct
MRMSRRAKLRCALPLALVAVVAAFAPAANAQLAVDYSLPMADAAWAANPNGSPPGANDWSCRPSSLHPYPVILVHGTFSNQRFTWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDAARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 400 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcacaactggcggtggactatagcctgccgatggcggacgctgcatgggcagccaatccgaatggcagcccaccgggtgcgaacgattggtcgtgtcgcccaagctccttgcacccttatccggtgattctggtccacggtacattcagcaaccagcgcttcacatggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgacgcggctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 401 - protein, 319 aa, synthetic construct
MRMSRRAKLRCALPLALVAVVAAFAPAANAQLAVDYSLPKALAAWAANPNGSPPGANDWSCRPSSLHPYPVILVHGTFSNQRFTWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDVARLLGPIGNAAVQLVTSFCPACGDMIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 402 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcacatggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgacgtggctcgcttgctcggccctattggcaatgcagccgttcagcttgtcaccagcttctgcccggcatgtggggatatgatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg